(19) United States
(12) Patent Application Publication
(10) Pub. No.: US 2014/0315744 A1
(43) Pub. Date: Oct. 23, 2014

(54) ASSESSMENT OF OOCYTE COMPETENCE BY DETECTING SPSB2 AND/OR TP53I3 GENE EXPRESSION

(71) Applicant: Gema Diagnostics, Inc., Ann Arbor, MI (US)

(72) Inventors: Dagan Wells, Oxford (GB); Pasquale Patrizio, Guilford, CT (US)

(21) Appl. No.: 14/351,768

(22) PCT Filed: Oct. 15, 2012
(86) PCT No.: PCT/US12/60296

§ 371 Date: Apr. 14, 2014

Related U.S. Application Data
(60) Provisional application No. 61/581,187, filed on Dec. 29, 2011.
(60) Provisional application No. 61/547,379, filed on Oct. 14, 2011.

(51) Int. Cl. C12Q 1/68; G01N 33/569

(52) U.S. Cl. CPC C12Q 1/6881; G01N 33/56966; C12Q 2600/158
USPC 506/9; 506/16; 506/18; 435/6.12; 435/6.11; 435/7.92; 435/7.1

(57) ABSTRACT

Methods are provided for evaluating an oocyte for fertilization and implantation comprising analysis of gene expression in cumulus cells wherein the detected genes or polypeptides include at least SPSB2 and TP53I3. For example, methods are provided for determining whether a cumulus cell expresses, or does not express, one or more of a group of markers identified as differently expressed between cumulus cells associated with chromosomally normal oocytes and cumulus cells associated with chromosomally abnormal oocytes. Methods are provided for the detection of marker expression of differentially expressed genes at the RNA level, as well as at the protein level.

### RELATED APPLICATIONS

This PCT application claims priority to U.S. provisional Ser. No. 61/547,379 filed on Oct. 14, 2011, and U.S. provisional Ser. No. 61/581,187 filed on Dec. 29, 2011. The contents of both provisional applications are incorporated by reference in their entirety herein.

### BACKGROUND OF THE INVENTION

Currently, there is no reliable commercially available genetic or non-genetic procedure for identifying whether a oocytes that are “pregnancy competent”, i.e., oocytes which when fertilized by natural or artificial means are capable of giving rise to embryos that in turn are capable of yielding viable offspring when transferred to an appropriate uterine environment (e.g., oocytes that have a normal chromosome competent).

Perhaps in part of the lack of a means for identifying pregnancy competent oocytes, the success rate for assisted reproductive technology (ART), pregnancy and birth rates following in vitro fertilization (IVF) attempts remain low. Subjective morphological parameters are still a primary criterion to select healthy embryos used for in IVF and ICSI programs. However, such criteria do not truly predict the competence of an embryo. Many studies have shown that a combination of several different morphologic criteria leads to more accurate embryo selection. Morphological criteria for embryo selection are assessed on the day of transfer, and are principally based on early embryonic cleavage (25-27 h post insemination), the number and size of blastomeres on day two, day three, or day five, fragmentation percentage and the presence of multi-nucleation in the 4 or 8 cell stage (Fenwick et al., Hum Reprod, 17, 407-12. (2002).

As embryos that result in pregnancy differ in their metabolic profiles compared to embryos that do not, some studies are trying to identify a molecular signature that can be detected by non-invasive evaluation of the embryo culture medium (Brison et al., Hum Reprod, 19, 2319-24. (2004); Gardner et al., Fertil Steril, 76, 1175-80. (2001); Sakkas and Gardner, Curr Opin Obstet Gynecol, 17, 283-8 (2005); Seli et al., Fertil Steril, 88, 1350-7. (2007); Zhu et al. Fertil Steril. (2007). Currently, when a woman undergoes preimplantation genetic screening (PGS) in a fertility clinic, doctors are trying to select an oocyte or an embryo that is healthy (e.g., normal karyotype and does not have any chromosomal abnormality). This currently requires a biopsy of the polar body or removal a cell from the embryo for screening. Both procedures are expensive, invasive and risk damage to the oocyte or embryo.

Cumulus cells (CCs) are differentiated granulosa cells that form layers surrounding the oocyte in antral follicles. Bi-directional communication is established between the CCs surrounding the oocyte via the formation of projections that pierce the zona pellucida and form gap junctions with the oocyte plasma membrane. These junctions enable the continuous exchange of proteins and metabolites between the two types of cells. Feuerstein, et al. (2007) Hum Reprod 22: 3069-3077. During the luteinizing hormone (LH) surge, CCs transmit a maturation-inducing signal from the extra-follicular environment to the enclosed oocyte, which in turn resumes meiosis, and this is only observed for pre-ovulatory follicles with sufficient LH receptors. Hillier (1994) Hum Reprod 9: 188-191. Additionally, in mice, it has been shown that correct meiotic spindle positioning is regulated via the cross-talk established between the oocyte and its surrounding CCs (cumulus cells). Barrett & Albertini (2010) J Assist Reprod Genet 27: 29-39.

Female meiosis is prone to chromosome malsegregation errors leading to aneuploidy, which increases dramatically with advancing age. Two main mechanisms have been described: the first involves the non-disjunction of entire chromosomes, observed during both meiotic divisions [Zenzes & Casper (1992) Hum Genet 88: 367-375], and the second involves the premature division of a chromosome into its two constituent chromatids (predivision), followed by their random segregation, upon completion of the first and/or second meiotic division. Angell (1991) Hum Genet 86: 383-387. The direct and very close relationship between advancing female age and increasing aneuploidy rates has been clearly and unequivocally demonstrated during the cytogenetic analysis of large numbers of human oocytes. Most such studies suggest that the expected oocyte aneuploidy rate for women under the age of 25 years is ˜5%, increasing to 10-25% by the early 30's and exceeding 50% in the oocytes of women over 40. Sandalinas, et al. (2002) Mol Hum Prod 8: 580-585; Kuliev, et al. (2003) RBM Online 6: 54-59; Pellestor, et al. (2003) Hum Genet 112: 195-203; Fragouli, et al. (2006) Hum Reprod 21: 2319-2328; Fragouli, et al. (2009) Reprod Biomed Online 19: 228-237; Fragouli, et al. (2010) Fertil Steril 94: 875-887.

Although genetic analyses, via preimplantation genetic screening (PGS), can reveal the presence of lethal chromosome anomalies in oocytes and embryos, the invasive nature of such procedures may have an adverse impact on embryonic viability and requires highly skilled embryologists, adding to the expense of such procedures. Thus, reproductive medicine would benefit greatly from a method capable of the noninvasive characterization and identification of those oocytes or embryos most likely to result in successful fertilization and implantation by measuring the level of marker expression associated with oocyte competence and oocyte incompetence.

### SUMMARY OF THE DISCLOSURE

The present invention contemplates a method of evaluating the competence of a mammalian oocyte for fertilization, or for implantation, or both wherein the methods comprise (i) obtaining a nucleic acid or polypeptide sample; (ii) determining the level of marker expression of at least one gene or polypeptide encoded thereby selected from the group of TP53I3 or SPSB2 in said sample; and (iii) comparing the level of marker expression TP53I3 or SPSB2 in the sample with a control or reference standard, wherein detecting differential marker expression between the sample and the control is indicative of the competence of an oocyte for implantation or fertilization.

In preferred embodiments, said TP53I3 gene is a human or non-human primate gene, e.g., at least 90, 95 or 100% identical to the TP53I3 gene having the e nucleic acid sequence of SEQ ID NO: 385, 387, or 389, or comprising the amino acid sequence of SEQ ID NO: 386, 388, or 390.

Also, in preferred embodiments the SPSB2 gene or polypeptide is a human or non-human primate gene or polypeptide, e.g., one at least 90, 95 or 100% identical to the SPSB2 gene having the nucleic acid sequence of SEQ ID NO: 252 or comprises a polypeptide at least 90, 95 or 100% identical to the amino acid sequence of SEQ ID NO: 351.

Also, the invention includes arrays for use in such detection methods, i.e., arrays comprising at least SPSB2 and TP53I3 genes or nucleic acids, primers, polypeptides or antibodies which specifically detect, amplify, or bind to SPSB2 and TP53I3 nucleic acids or polypeptides.

Also, the invention specifically includes arrays for use in such detection methods, that comprise primers which amplify SPSB2 and TP53I3 nucleic acids.

Also, the invention specifically includes arrays for use in such detection methods, that comprise antibodies or nucleic acids which specifically bind SPSB2 and TP53I3 polypeptides.

Also, the invention specifically includes arrays for use in such detection methods, that comprise nucleic acids or polypeptides that are at least 90% identical to the TP53I3 nucleic acid sequence of SEQ ID NO: 385, 387, or 389, or the amino acid sequence of SEQ ID NO: 386, 388, or 390 and/or to the SPSB2 nucleic acid sequence of SEQ ID NO: 252 and/or the amino acid sequence of SEQ ID NO: 351.

Also, the invention specifically includes arrays for use in such detection methods, that comprise nucleic acids or polypeptides that are at least 95% identical to the TP53I3 nucleic acid sequence of SEQ ID NO: 385, 387, or 389, or the amino acid sequence of SEQ ID NO: 386, 388, or 390 or to the SPSB2 nucleic acid sequence of SEQ ID NO: 252 or the amino acid sequence of SEQ ID NO: 351.

Also, the invention specifically includes arrays for use in such detection methods, that comprise nucleic acids or polypeptides that are identical to the TP53I3 nucleic acid sequence of SEQ ID NO: 385, 387, or 389, or the amino acid sequence of SEQ ID NO: 386, 388, or 390, and to the SPSB2 nucleic acid sequence of SEQ ID NO: 252 and/or the amino acid sequence of SEQ ID NO: 351.

Also, the invention specifically includes arrays for use in such detection methods, that comprise nucleic acid primers that amplify the TP53I3 nucleic acid sequence of SEQ ID NO: 385, 387, or 389 and/or the SPSB2 nucleic acid sequence of SEQ ID NO: 252.

Also, the invention specifically includes arrays for use in such detection methods, wherein said TP53I3 gene are human or non-human primate genes and said nucleic acid, primer, polypeptide or antibody is a human or non-human primate gene, or a nucleic acid, primer, polypeptide or an antibody that specifically amplifies or specifically binds to said human or non-human primate gene, nucleic acid or polypeptide.

In some embodiments the method further comprises determining in a sample the level of markers expression in addition to SPSB2 and TP53I3 selected from at least one nucleic acid selected from the group of nucleic acids exemplified by SEQ ID NOS:1-92 and 183-292, and comparing the level of marker expression in the sample with a control or reference standard, wherein detecting differential marker expression between the sample and the control may be indicative of the competence of the oocyte for fertilization, or for implantation, or for both.

It may be an aspect of the invention that the sample may be derived from an oocyte, follicular fluid, cumulus cell, or culture medium. The control or reference standard may be derived from an oocyte competent for implantation, an oocyte not competent for implantation, an oocyte competent for fertilization, an oocyte not competent for fertilization, a chromosomally normal oocyte, a chromosomally abnormal oocyte, follicular fluid associated with an oocyte competent for implantation, follicular fluid associated with an oocyte not competent for implantation, follicular fluid associated with an oocyte competent for fertilization, follicular fluid associated with an oocyte not competent for fertilization, follicular fluid associated with a chromosomally normal oocyte, follicular fluid associated with a chromosomally abnormal oocyte, culture medium associated with an oocyte competent for implantation, culture medium associated with an oocyte not competent for implantation, culture medium associated with an oocyte competent for fertilization, culture medium associated with an oocyte not competent for fertilization, culture medium associated with a chromosomally normal oocyte, culture medium associated with a chromosomally abnormal oocyte, a cumulus cell associated with an oocyte competent for implantation, a cumulus cell associated with an oocyte not competent for implantation, a cumulus cell associated with an oocyte competent for fertilization, a cumulus cell associated with an oocyte not competent for fertilization, a cumulus cell associated with a chromosomally normal oocyte, a cumulus cell associated with a chromosomally abnormal oocyte, follicular fluid associated with a cumulus cell associated with an oocyte competent for implantation, follicular fluid associated with a cumulus cell associated with an oocyte not competent for implantation, follicular fluid associated with a cumulus cell associated with an oocyte competent for fertilization, follicular fluid associated with a cumulus cell associated with an oocyte not competent for fertilization, follicular fluid associated with a cumulus cell associated with a chromosomally normal oocyte, follicular fluid associated with a cumulus cell associated with a chromosomally abnormal oocyte, culture medium associated with a cumulus cell associated with an oocyte competent for implantation, culture medium associated with a cumulus cell associated with an oocyte not competent for implantation, culture medium associated with a cumulus cell associated with an oocyte competent for fertilization, culture medium associated with a cumulus cell associated with an oocyte not competent for fertilization, culture medium associated with a cumulus cell associated with a chromosomally normal oocyte, culture medium associated with a cumulus cell associated with a chromosomally abnormal oocyte or combinations thereof.

In one aspect, the level of marker expression determined in the sample may be at least about 20% different from the level of marker expression determined in the control or reference standard. In one aspect, the level of marker expression may be detected by nucleic acid microarray, Northern blot, or reverse transcription PCR. In another aspect, the level of marker expression may be detected by Western blot, immunoassay, enzyme-linked immunosorbent assay, protein microarray or FACS analysis.

It may be an aspect of the invention that the cumulus cell are human, but the cumulus cell may also be of a mammal.

In another aspect, the invention comprises an array of nucleic acid probes immobilized on a solid support, the probe set comprising a plurality of probes including those specific to least SPSB2 and TP53I3 genes, each probe comprising a segment of at least twenty nucleotides exactly complementary to a subsequence of a set of reference sequences, wherein the set of reference sequences comprises probes including those specific to least SPSB2 and TP53I3 genes and may in addition include sequence selected from SEQ ID NOS:1-92.

In another aspect, the invention comprises an array of nucleic acid probes immobilized on a solid support, the probe set comprising a plurality of probes, each probe comprising a segment of at least twenty nucleotides exactly complementary to a subsequence of a set of reference sequences, wherein the set of reference sequences comprises SEQ ID NOS:183-282.

In one embodiment, a method of evaluating the competence of a mammalian oocyte for implantation may comprise determining the level of marker expression of at least one nucleic acid selected from the group of nucleic acids consisting of SEQ ID NOS: 1-92 in said sample; and comparing the level of marker expression in the sample with a control or reference standard, wherein detecting differential marker expression between the sample and the control may be indicative of the competence of the oocyte for implantation.

In one embodiment, a method of evaluating the competence of a mammalian oocyte for implantation may comprise determining the level of marker expression of at least one nucleic acid that encodes SPSB2 and TP53I3 or polypeptide encoded thereby and optionally another gene or amino acid sequence selected from the group of amino acids consisting of SEQ ID NOS: 93-182 in said sample; and comparing the level of marker expression in the sample with a control or reference standard, wherein detecting differential marker expression between the sample and the control may be indicative of the competence of the oocyte for implantation.

In one embodiment, a method of evaluating the competence of a mammalian oocyte for implantation may comprise determining the level of marker expression of at least one nucleic acid that encodes SPSB2 and TP53I3 or polypeptide encoded thereby and optionally another amino acid or nucleic acid including one selected from the group of amino acids consisting of SEQ ID NOS: 93-182 in said sample; and comparing the level of marker expression in the sample with a control or reference standard, wherein detecting differential marker expression between the sample and the control may be indicative of the competence of the oocyte for implantation.

In one embodiment, a method of evaluating the competence of a mammalian oocyte for fertilization may comprise determining the level of marker expression of at least one nucleic acid that encodes SPSB2 and TP53I3 or polypeptide encoded thereby and optionally another nucleic acid selected from the group of nucleic acids consisting of SEQ ID NOS: 1-92 in said sample; and comparing the level of marker expression in the sample with a control or reference standard, wherein detecting differential marker expression between the sample and the control may be indicative of the competence of the oocyte for fertilization.

In one embodiment, a method of evaluating the competence of a mammalian oocyte for fertilization may comprise determining the level of marker expression of at least one nucleic acid that encodes SPSB2 and TP53I3 or polypeptide encoded thereby and optionally another nucleic acid that encodes an amino acid sequence selected from the group of amino acids consisting of SEQ ID NOS: 93-182 in said sample; and comparing the level of marker expression in the sample with a control or reference standard, wherein detecting differential marker expression between the sample and the control may be indicative of the competence of the oocyte for fertilization. In one embodiment, a method of evaluating the competence of a mammalian oocyte for fertilization may comprise determining the level of marker expression of at least one nucleic acid that encodes SPSB2 and TP53I3 or polypeptide encoded thereby and optionally another amino acid selected from the group of amino acids consisting of SEQ ID NOS: 93-182 in said sample; and comparing the level of marker expression in the sample with a control or reference standard, wherein detecting differential marker expression between the sample and the control may be indicative of the competence of the oocyte for fertilization. In another embodiment, the method may further comprise obtaining a sample. In another embodiment, the sample may be a nucleic acid sample or an amino acid sample.

In one embodiment, the sample may be derived from an oocyte. In another embodiment, the control or reference standard may be derived from one of the group consisting of an oocyte competent for implantation, a chromosomally normal oocyte, an oocyte not competent for implantation, and a chromosomally abnormal oocyte.

In one embodiment, the sample may be derived from follicular fluid. In another embodiment, the control or reference standard may be derived from one of the group consisting of follicular fluid associated with an oocyte competent for implantation, follicular fluid associated with a chromosomally normal oocyte, follicular fluid associated with an oocyte not competent for implantation and follicular fluid associated with a chromosomally abnormal oocyte.

In one embodiment, the sample may be derived from culture medium. In another embodiment, the control or reference standard may be derived from one of the group consisting of culture medium associated with an oocyte competent for implantation, culture medium associated with a chromosomally normal oocyte, culture medium associated with an oocyte not competent for implantation, and culture medium associated with a chromosomally abnormal oocyte.

In one embodiment, a method of evaluating the competence of a mammalian oocyte for implantation may comprise determining the level of marker expression of at least one nucleic acid that encodes SPSB2 and TP53I3 or polypeptide encoded thereby and optionally another nucleic acid selected from the group of nucleic acids consisting of SEQ ID NOS: 183-282 in said sample; and comparing the level of marker expression in the sample with a control or reference standard, wherein detecting differential marker expression between the sample and the control may be indicative of the competence of the oocyte for implantation.

In one embodiment, a method of evaluating the competence of a mammalian oocyte for implantation may comprise determining the level of marker expression of at least one nucleic acid that encodes SPSB2 and TP53I3 or polypeptide encoded thereby and optionally another nucleic acid that encodes an amino acid sequence selected from the group of amino acids consisting of SEQ ID NOS: 183-282 in said sample; and comparing the level of marker expression in the sample with a control or reference standard, wherein detecting differential marker expression between the sample and the control may be indicative of the competence of the oocyte for implantation.

In one embodiment, a method of evaluating the competence of a mammalian oocyte for implantation may comprise determining the level of marker expression of at least one of at least one nucleic acid that encodes SPSB2 and TP53I3 or polypeptide encoded thereby and optionally another amino acid selected from the group of amino acids consisting of SEQ ID NOS: 283-390 in said sample; and comparing the level of marker expression in the sample with a control or reference standard, wherein detecting differential marker expression between the sample and the control may be indicative of the competence of the oocyte for implantation.

In one embodiment, a method of evaluating the competence of a mammalian oocyte for fertilization may comprise determining the level of marker expression of at least one nucleic acid that encodes SPSB2 and TP53I3 or polypeptide encoded thereby and optionally another one nucleic acid selected from the group of nucleic acids consisting of SEQ ID NOS: 183-282 in said sample; and comparing the level of marker expression in the sample with a control or reference standard, wherein detecting differential marker expression between the sample and the control may be indicative of the competence of the oocyte for fertilization.

In one embodiment, a method of evaluating the competence of a mammalian oocyte for fertilization may comprise determining the level of marker expression of at least one nucleic acid that encodes SPSB2 and TP53I3 or polypeptide encoded thereby and optionally another nucleic acid that encodes an amino acid sequence selected from the group of amino acids consisting of SEQ ID NOS: 183-282 in said sample; and comparing the level of marker expression in the sample with a control or reference standard, wherein detecting differential marker expression between the sample and the control may be indicative of the competence of the oocyte for fertilization.

In one embodiment, a method of evaluating the competence of a mammalian oocyte for fertilization may comprise determining the level of marker expression of at least one nucleic acid that encodes SPSB2 and TP53I3 or polypeptide encoded thereby and optionally another amino acid selected from the group of amino acids consisting of SEQ ID NOS: 283-390 in said sample; and comparing the level of marker expression in the sample with a control or reference standard, wherein detecting differential marker expression between the sample and the control may be indicative of the competence of the oocyte for fertilization.

In another embodiment, the method may further comprise obtaining a sample. In another embodiment, the sample may be a nucleic acid sample or an amino acid sample.

In one embodiment, the sample may be derived from a cumulus cell. In one embodiment, the control or reference standard may be derived from one of the group consisting of a cumulus cell associated with an oocyte competent for implantation, a cumulus cell associated with a chromosomally normal oocyte, a cumulus cell associated with an oocyte not competent for implantation and a cumulus cell associated with a chromosomally abnormal oocyte.

In one embodiment, the sample may be derived from follicular fluid. In another embodiment, the control or reference standard may be derived from one of the group consisting of follicular fluid associated with a cumulus cell associated with an oocyte competent for implantation, follicular fluid associated with a cumulus cell associated with a chromosomally normal oocyte, follicular fluid associated with a cumulus cell associated with an oocyte not competent for implantation and follicular fluid associated with a cumulus cell associated with a chromosomally abnormal oocyte.

In one embodiment, the sample may be derived from culture medium. In another embodiment, the control or reference standard may be derived from one of the group consisting of culture medium associated with a cumulus cell associated with an oocyte competent for implantation, culture medium associated with a cumulus cell associated with a chromosomally normal oocyte, culture medium associated with a cumulus cell associated with an oocyte not competent for implantation, and culture medium associated with a cumulus cell associated with a chromosomally abnormal oocyte.

In one embodiment, the level of marker expression determined in the sample may be at least 20% different from the level of marker expression determined in the control or reference standard. In another embodiment, the level of marker expression may be detected by nucleic acid microarray, Northern blot, real-time PCR, or reverse transcription PCR. In another embodiment, the level of marker expression may be detected by Western blot, immunoassay (e.g., enzyme-linked immunosorbent assay), protein microarray, or FACS analysis.

In one embodiment, the mammalian oocyte may be a domesticated mammal. In another embodiment, the mammalian oocyte may be a human, cat, dog, cow, pig, goat, sheep, or camel. In a further embodiment, the mammalian oocyte may be a human oocyte.

In one embodiment, the method of evaluating the competence of a mammalian oocyte for implantation may comprise determining the level of marker expression of at least one gene selected from the group consisting of at least one nucleic acid that encodes SPSB2 and TP53I3 or polypeptide encoded thereby and optionally another selected from B3GALNT2, C22orf29, CCL16, DCBLD1, DCC1, DHX9, OTUD5, RBBP6, SEPT11, SLC25A36, and TACSTD2, Unassigned (helicase), in said sample; and comparing the level of marker expression in the sample with a control or reference standard, wherein detecting differential marker expression between the sample and the control may be indicative of the competence of the oocyte for implantation. In another embodiment, the method of evaluating the competence of a mammalian oocyte for fertilization may comprise determining the level of marker expression of at least one gene selected from the group consisting of at least one nucleic acid that encodes SPSB2 and TP53I3 or polypeptide encoded thereby and optionally another gene or polypeptide selected from B3GALNT2, C22orf29, CCL16, DCBLD1, DCC1, DHX9, OTUD5, RBBP6, SEPT11, SLC25A36, TACSTD2, Unassigned (helicase), in said sample; and comparing the level of marker expression in the sample with a control or reference standard, wherein detecting differential marker expression between the sample and the control may be indicative of the competence of the oocyte for fertilization. In another embodiment, the method may further comprise obtaining a sample. In another embodiment, the sample may be a nucleic acid sample or an amino acid sample. In a further embodiment, the sample may be derived from a cumulus cell. In a further embodiment, the sample may be derived from an oocyte. In a further embodiment, the sample may be derived from a polar body. In a further embodiment, the sample may be a nucleic acid sample. In a further embodiment, the sample may be an amino acid sample.

In one embodiment, the control or reference standard may be derived from one of the group consisting of a cumulus cell associated with an oocyte competent for implantation, a cumulus cell associated with a chromosomally normal oocyte, a cumulus cell associated with an oocyte not competent for implantation and a cumulus cell associated with a chromosomally abnormal oocyte.

In one embodiment, the sample may be derived from follicular fluid. In one embodiment, the control or reference standard may be derived from one of the group consisting of follicular fluid associated with a cumulus cell associated with an oocyte competent for implantation, follicular fluid associated with a cumulus cell associated with a chromosomally normal oocyte, follicular fluid associated with a cumulus cell associated with an oocyte not competent for implantation and follicular fluid associated with a cumulus cell associated with a chromosomally abnormal oocyte.

In one embodiment, the sample may be derived from culture medium. In one embodiment, the control or reference standard may be derived from one of the group consisting of culture medium associated with a cumulus cell associated with an oocyte competent for implantation, culture medium associated with a cumulus cell associated with a chromosomally normal oocyte, culture medium associated with a cumulus cell associated with an oocyte not competent for implantation, and culture medium associated with a cumulus cell associated with a chromosomally abnormal oocyte.

In one embodiment, the level of marker expression determined in the sample may be at least 20% different from the level of marker expression determined in the control or reference standard. In another embodiment, the level of marker expression may be detected by nucleic acid microarray, cytogenetic analysis (aCGH), real-time PCR, TLDA, Northern blot, or reverse transcription PCR. In another embodiment, the level of marker expression may be detected by Western blot, immunoassay (e.g., enzyme-linked immunosorbent assay), protein microarray, or FACS analysis.

In one embodiment, the mammalian oocyte may be a domesticated mammal. In another embodiment, the mammalian oocyte may be a human.

In one embodiment, the method detects chromosomal abnormalities in the oocyte including but not limited to aneuploidy, translocation, deletion, and duplication.

In one embodiment, B3GALNT2 may be encoded by the nucleic acid sequence of SEQ ID NO: 185 or comprises the amino acid sequence of SEQ ID NO: 285. In another embodiment, C22orf29 may be encoded by the nucleic acid sequence of SEQ ID NO: 381 or comprises the amino acid sequence of SEQ ID NO: 382. In another embodiment, CCL16 may be encoded by the nucleic acid sequence of SEQ ID NO: 187 or comprises the amino acid sequence of SEQ ID NO: 287. In another embodiment, DCBLD1 may be encoded by the nucleic acid sequence of SEQ ID NO: 291 or comprises the amino acid sequence of SEQ ID NO: 291. In another embodiment, DHX9 may be encoded by the nucleic acid sequence of SEQ ID NO: 188 or comprises the amino acid sequence of SEQ ID NO: 288. In another embodiment, OTUD5 may be encoded by the nucleic acid sequence of SEQ ID NO: 193 or comprises the amino acid sequence of SEQ ID NO: 293. In another embodiment, RBBP6 may be encoded by the nucleic acid sequence of SEQ ID NO: 184 or comprises the amino acid sequence of SEQ ID NO: 284. In another embodiment, SEPT11 may be encoded by the nucleic acid sequence of SEQ ID NO: 192 or comprises the amino acid sequence of SEQ ID NO: 292. In another embodiment, SLC25A36 may be encoded by the nucleic acid sequence of SEQ ID NO: 190 or comprises the amino acid sequence of SEQ ID NO: 290. In another embodiment, SPSB2 may be encoded by the nucleic acid sequence of SEQ ID NO: 252 or comprises the amino acid sequence of SEQ ID NO: 351. In another embodiment, TACSTD2 may be encoded by the nucleic acid sequence of SEQ ID NO: 383 or comprises the amino acid sequence of SEQ ID NO: 384. In another embodiment, TP53I3 may be encoded by the nucleic acid sequence of SEQ ID NO: 385, 387, or 389, or comprises the amino acid sequence of SEQ ID NO: 386, 388, or 390.

In one embodiment, an array may comprise at least one gene selected from the group consisting of B3GALNT2, C22orf29, CCL16, DCBLD1, DCC1, DHX9, OTUD5, RBBP6, SEPT11, SLC25A36, SPSB2, TACSTD2, Unassigned (helicase), and TP53I3. In another embodiment, the array may comprise at least two of said genes. In one embodiment, B3GALNT2 may be encoded by the nucleic acid sequence of SEQ ID NO: 185 or comprises the amino acid sequence of SEQ ID NO: 285.

In one embodiment, C22orf29 may be encoded by the nucleic acid sequence of SEQ ID NO: 381 or comprises the amino acid sequence of SEQ ID NO: 382. In one embodiment, CCL16 may be encoded by the nucleic acid sequence of SEQ ID NO: 187 or comprises the amino acid sequence of SEQ ID NO: 287. In one embodiment, DCBLD1 may be encoded by the nucleic acid sequence of SEQ ID NO: 291 or comprises the amino acid sequence of SEQ ID NO: 291. In one embodiment, DHX9 may be encoded by the nucleic acid sequence of SEQ ID NO: 188 or comprises the amino acid sequence of SEQ ID NO: 288. In one embodiment, OTUD5 may be encoded by the nucleic acid sequence of SEQ ID NO: 193 or comprises the amino acid sequence of SEQ ID NO: 293. In one embodiment, RBBP6 may be encoded by the nucleic acid sequence of SEQ ID NO: 184 or comprises the amino acid sequence of SEQ ID NO: 284. In one embodiment, SEPT11 may be encoded by the nucleic acid sequence of SEQ ID NO: 192 or comprises the amino acid sequence of SEQ ID NO: 292. In one embodiment, SLC25A36 may be encoded by the nucleic acid sequence of SEQ ID NO: 190 or comprises the amino acid sequence of SEQ ID NO: 290. In one embodiment, SPSB2 may be encoded by the nucleic acid sequence of SEQ ID NO: 252 or comprises the amino acid sequence of SEQ ID NO: 351. In one embodiment, TACSTD2 may be encoded by the nucleic acid sequence of SEQ ID NO: 383 or comprises the amino acid sequence of SEQ ID NO: 384. In one embodiment, TP53I3 may be encoded by the nucleic acid sequence of SEQ ID NO: 385, 387, or 389, or comprises the amino acid sequence of SEQ ID NO: 386, 388, or 390.

### BRIEF DESCRIPTION OF THE DRAWINGS

FIG. 1 depicts an exemplary methodology for the assessment of the competence of oocytes for fertilization and/or implantation.

FIG. 2 depicts the expression of SPSB2 in cumulus cells (CCs) related to pregnancy outcome.

FIG. 3 contains the expression patterns seen for SPSB2 and TP53I3 in CCs of normal and aneuploid oocytes.

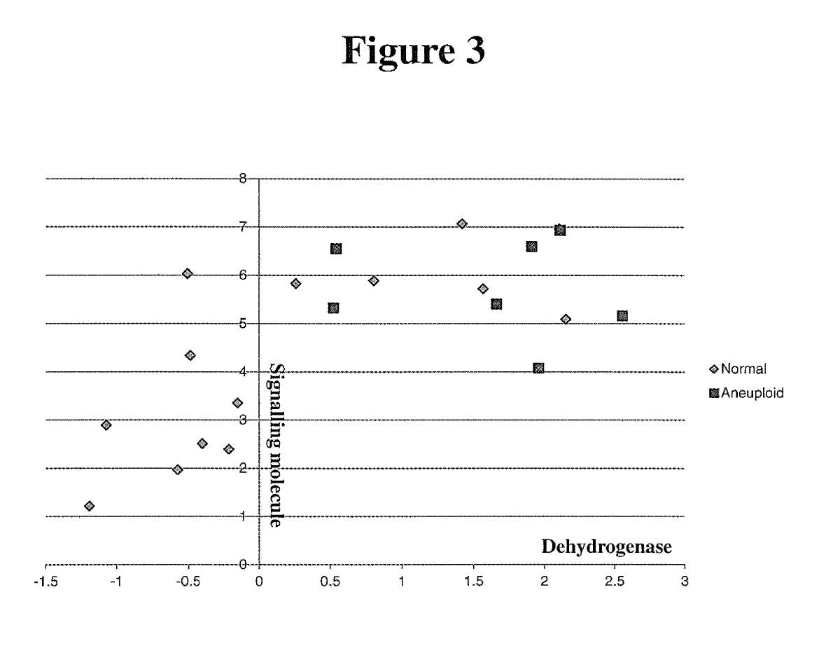

FIG. 4 shows the differences in expression between the two sample groups approached statistical significance (P=0.054 two tailed t-test), and are illustrated in the box plot.

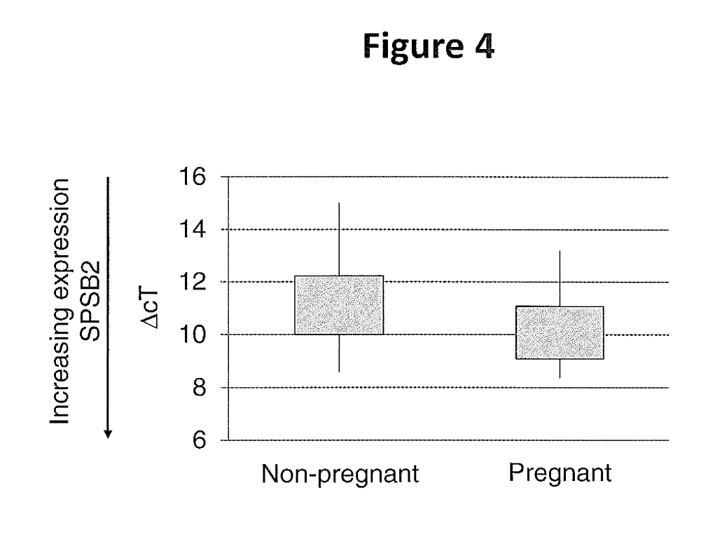

FIG. 5 contains real-time PCR analysis of SPSB2 and TP53I3 in cumulus cells associated with chromosomally normal or aneuploid oocytes.

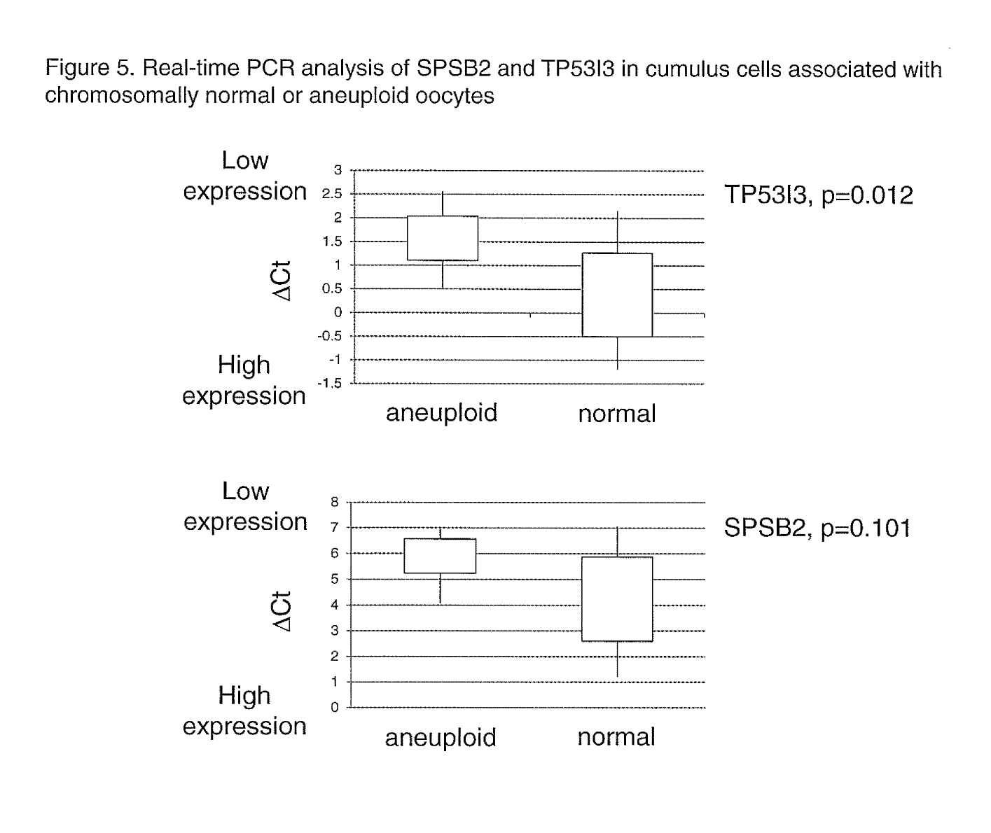

### DETAILED DESCRIPTION OF THE INVENTION

### Definitions

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to at least one proteins and equivalents thereof known to those skilled in the art, and so forth. All technical and scientific terms used herein have the same meaning as commonly understood to one of ordinary skill in the art to which this invention belongs unless clearly indicated otherwise. The articles “a” and “an” are used herein to refer to one or to more than one (i.e. to at least one) of the grammatical object of the article. By way of example, “an element” means one element or more than one element.

Allele as used herein broadly refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variants”, “polymorphisms”, or “mutations”.

Amplification as used herein, refers broadly to the amplification of polynucleotide sequences is the in vitro production of multiple copies of a particular nucleic acid sequence. The amplified sequence is usually in the form of DNA. A variety of techniques for carrying out such amplification are known in the art. See, e.g., Van Brunt (1990) Bio/Technol. 8(4): 291-294. Polymerase chain reaction or PCR is a prototype of nucleic acid amplification, and use of PCR herein should be considered exemplary of other suitable amplification techniques.

Antibody as used herein refers broadly to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies. Harlow & Lane (1999) Using Antibodies: A Laboratory Manual Cold Spring Harbor Laboratory Press; Houston, et al. (1988) Proc. Natl. Acad. Sci. USA 85:5879-5883; Bird, et al. (1988) Science 242:423-426.

Array as used herein refers broadly to a support, preferably solid, with nucleic acid probes attached to the support. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., 1991, Science, 251:767-777, each of which is incorporated by reference in its entirety for all purposes.

Aneuploidy as used herein refers broadly to an abnormal number of chromosomes, and is a type of chromosome abnormality. For example, humans have 46 chromosomes comprising 23 pairs of chromosomes, an example of aneuploidy being trisomy 21, where the patient has three copies of chromosome 21. Further examples of aneuploidy include but are not limited to monosomy, where the patient may lack one chromosome of the normal complement, and tetrasomy and pentasomy, where the patient has four or five copies, respectively, of a chromosome.

Characteristic level of expression of a cumulus gene as used herein refers broadly to a particular detected expressed nucleic acid sequence or polypeptide means that the particular gene or polypeptide is expressed at levels which are substantially similar to the levels observed in cumulus cells that are associated with a normal cumulus cell or one associated with a normal or developmentally competent oocyte.

Chromosome as used herein refers broadly to the heredity-bearing gene carrier of a cell which is derived from chromatin and which comprises DNA and protein components (especially histones). The conventional internationally recognized individual human genome chromosome numbering system is employed herein. The size of an individual chromosome can vary from one type to another within a given multi-chromosomal genome and from one genome to another. In the case of the human genome, the entire DNA mass of a given chromosome is usually greater than about 100,000,000 bp. For example, the size of the entire human genome is about 3×10⁹ bp. The largest chromosome, chromosome no. 1, contains about 2.4×10⁸ by while the smallest chromosome, chromosome no. 22, contains about 5.3×10⁷ bp.

Chromosomal region as used herein refers broadly to a portion of a chromosome. The actual physical size or extent of any individual chromosomal region can vary greatly. The term “region” is not necessarily definitive of a particular one or more genes because a region need not take into specific account the particular coding segments (exons) of an individual gene.

Polymorphism refers to the occurrence of two or more genetically determined alternative sequences or alleles in a population. A polymorphic marker or site is the locus at which divergence occurs. A polymorphism may comprise one or more base changes, an insertion, a repeat, or a deletion. A polymorphic locus may be as small as one base pair. The first identified allelic form is arbitrarily designated as the reference form and other allelic forms are designated as alternative or variant alleles. The allelic form occurring most frequently in a selected population is sometimes referred to as the wildtype form. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

Single nucleotide polymorphisms (SNPs) are positions at which two alternative bases occur at appreciable frequency (about at least 1%) in a given population. A SNP may arise due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

Control or reference standard as used herein refers broadly to a material comprising none, or a normal, low, or high level of one of more of the marker expression products of one or more the sequences listed herein in the accompanying sequence listing, such that the control or reference standard may serve as a comparator against which a sample can be compared. By way of non-limiting examples, a control or reference standard may include all or a part of any of an oocyte competent for implantation, an oocyte not competent for implantation, an oocyte competent for fertilization, an oocyte not competent for fertilization, a chromosomally normal oocyte, a chromosomally abnormal oocyte, follicular fluid associated with an oocyte competent for implantation, follicular fluid associated with an oocyte not competent for implantation, follicular fluid associated with an oocyte competent for fertilization, follicular fluid associated with an oocyte not competent for fertilization, follicular fluid associated with a chromosomally normal oocyte, follicular fluid associated with a chromosomally abnormal oocyte, culture medium associated with an oocyte competent for implantation, culture medium associated with an oocyte not competent for implantation, culture medium associated with an oocyte competent for fertilization, culture medium associated with an oocyte not competent for fertilization, culture medium associated with a chromosomally normal oocyte, culture medium associated with a chromosomally abnormal oocyte, a cumulus cell associated with an oocyte competent for implantation, a cumulus cell associated with an oocyte not competent for implantation, a cumulus cell associated with an oocyte competent for fertilization, a cumulus cell associated with an oocyte not competent for fertilization, a cumulus cell associated with a chromosomally normal oocyte, a cumulus cell associated with a chromosomally abnormal oocyte, follicular fluid associated with a cumulus cell associated with an oocyte competent for implantation, follicular fluid associated with a cumulus cell associated with an oocyte not competent for implantation, follicular fluid associated with a cumulus cell associated with an oocyte competent for fertilization, follicular fluid associated with a cumulus cell associated with an oocyte not competent for fertilization, follicular fluid associated with a cumulus cell associated with a chromosomally normal oocyte, follicular fluid associated with a cumulus cell associated with a chromosomally abnormal oocyte, culture medium associated with a cumulus cell associated with an oocyte competent for implantation, culture medium associated with a cumulus cell associated with an oocyte not competent for implantation, culture medium associated with a cumulus cell associated with an oocyte competent for fertilization, culture medium associated with a cumulus cell associated with an oocyte not competent for fertilization, culture medium associated with a cumulus cell associated with a chromosomally normal oocyte, culture medium associated with a cumulus cell associated with a chromosomally abnormal oocyte or combinations thereof.

Cumulus cell as used herein refers broadly to a cell comprised in a mass of cells that surrounds an oocyte. This is an example of an “oocyte associated cell”. These cells are believed to be involved in providing an oocyte some of its nutritional and or other requirements that are necessary to yield an oocyte which upon fertilization is “pregnancy competent” (Buccione, R., Schroeder, A. C., and Eppig, J. J. (1990). Interactions between somatic cells and germ cells throughout mammalian oogenesis. Biol Reprod 43, 543-547.)

Differential gene expression as used herein refers broadly to genes the expression of which varies within a tissue of interest; herein preferably a cell associated with an oocyte, e.g., a cumulus cell.

Determining the level of marker expression as used herein refers broadly to an assessment of the degree of expression of a marker in a sample at the nucleic acid or protein level, using technology available to the skilled artisan to detect a sufficient portion of any marker expression product (including nucleic acids and proteins) of any one of the sequences listed herein in the accompanying sequence listing, such that the sufficient portion of the marker expression product detected is indicative of the expression of any one of the sequences listed herein in the accompanying sequence listing.

Genome as used herein refers broadly to all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

Genotyping as used herein refers broadly to the determination of the genetic information an individual carries at one or more positions in the genome. For example, genotyping may comprise the determination of which allele or alleles an individual carries for a single SNP or the determination of which allele or alleles an individual carries for a plurality of SNPs. For example, a particular nucleotide in a genome may be an A in some individuals and a C in other individuals. Those individuals who have an A at the position have the A allele and those who have a C have the C allele. A polymorphic location may have two or more possible alleles and the array may be designed to distinguish between all possible combinations.

Heavy chain antibody or heavy chain antibodies as used herein refers broadly to immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

Homologous as used herein refers broadly to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

Homology, as used herein, refers broadly to a degree of similarity between a nucleic acid sequence and a reference nucleic acid sequence or between a polypeptide sequence and a reference polypeptide sequence. Homology may be partial or complete. Complete homology indicates that the nucleic acid or amino acid sequences are identical. A partially homologous nucleic acid or amino acid sequence is one that is not identical to the reference nucleic acid or amino acid sequence. The degree of homology can be determined by sequence comparison. The term “sequence identity” may be used interchangeably with “homology.” As used herein, “homology” is used synonymously with “identity.” In addition, when the term “homology” is used herein to refer to the nucleic acids and proteins, it should be construed to be applied to homology at both the nucleic acid and the amino acid levels. The determination of percent identity between two nucleotide or amino acid sequences can be accomplished using a mathematical algorithm. For example, a mathematical algorithm useful for comparing two sequences is the algorithm of Karlin and Altschul (1990) Proc. Natl. Acad. Sci. USA 87: 2264-2268), modified as in Karlin and Altschul (1993) Proc. Natl. Acad. Sci. USA 90: 5873-5877. This algorithm is incorporated into the NBLAST and XBLAST programs of Altschul, et al. (1990) J. Mol. Biol. 215: 403-410, and can be accessed, for example, at the National Center for Biotechnology Information (NCBI) world wide web site having the universal resource locator www.ncbi.nlm.nih.gov/BLAST/. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein.

To obtain gapped alignments for comparison purposes, Gapped BLAST can be utilized as described in Altschul, et al. (1997) Nucleic Acids Res. 25: 3389-3402. Alternatively, PSI-Blast or PHI-Blast can be used to perform an iterated search which detects distant relationships between molecules (id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See www.ncbi.nlm.nih.gov. The percent identity between two sequences can be determined using techniques similar to those described above, with or without allowing gaps. In calculating percent identity, typically exact matches are counted.

Isolated, as used herein, refers broadly to material removed from its original environment in which it naturally occurs, and thus is altered by the hand of man from its natural environment. Isolated material may be, for example, exogenous nucleic acid included in a vector system, exogenous nucleic acid contained within a host cell, or any material which has been removed from its original environment and thus altered by the hand of man (e.g., “isolated antibody”).

Mammal, as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, camels, chimpanzees, chinchillas, cattle, dogs, goats, gorillas, hamsters, horses, humans, lemurs, llamas, mice, non-human primates, pigs, rats, sheep, shrews, squirrels, and tapirs. Mammals include but are not limited to bovine, canine, equine, feline, murine, ovine, porcine, primate, and rodent species. Domesticated mammals refer broadly to any mammalian species domesticated including but not limited to cattle, sheep, pigs, horses, camels, llamas, and goats. Mammal also includes any and all those listed on the Mammal Species of the World maintained by the National Museum of Natural History, Smithsonian Institution in Washington D.C.

Method for detecting differential expressed genes as used herein refers broadly to any known method for quantitatively evaluating differential gene expression using a probe that specifically detects for the expressed gene transcript or encoded polypeptide. Examples of such methods include indexing differential display reverse transcription polymerase chain reaction (DDRT-PCR; Mahadeva et al, 1998, J. Mol. Biol. 284:1391-1318; WO 94/01582; subtractive mRNA hybridization (See Advanced Mol. Biol.; R. M. Twyman (1999) Bios Scientific Publishers, Oxford, p. 334, the use of nucleic acid arrays or microarrays (see Nature Genetics, 1999, vol. 21, Suppl. 1061) and the serial analysis of gene expression. (SAGE) See e.g., Valculesev et al, Science (1995) 270:484-487) and real time PCR (RT-PCR). For example, differential levels of a transcribed gene in an oocyte cell can be detected by use of Northern blotting, and/or RT-PCR. A referred method is the CRL amplification protocol refers to the novel total RNA amplification protocol disclosed in Applicant's earlier applications that combines template-switching PCR and T7 based amplification methods. This protocol is well suited for samples wherein only a few cells or limited total RNA is available.

Marker expression as used herein refers broadly to the transcription, translation, post-translation modification, and phenotypic manifestation of a gene, including all aspects of the transformation of information encoded in a gene into RNA or protein. By way of non-limiting example, marker expression includes transcription into messenger RNA (mRNA) and translation into protein, as well as transcription into types of RNA such as transfer RNA (tRNA) and ribosomal RNA (rRNA) that are not translated into protein.

Microarray analysis as used herein refers broadly to the quantification of the expression levels of specific genes in a particular sample, e.g., tissue or cell sample.

Match, perfect match, perfect match probe, or perfect match control as used herein refers broadly to a nucleic acid that has a sequence that is perfectly complementary to a particular target sequence. The nucleic acid is typically perfectly complementary to a portion (subsequence) of the target sequence. A perfect match (PM) probe can be a “test probe”, a “normalization control” probe, an expression level control probe and the like. A perfect match control or perfect match is, however, distinguished from a “mismatch” or “mismatch probe.”

Mismatch, mismatch control, or mismatch probe as used herein refers broadly to a nucleic acid whose sequence is not perfectly complementary to a particular target sequence. As a non-limiting example, for each mismatch (MM) control in a high-density probe array there typically exists a corresponding perfect match (PM) probe that is perfectly complementary to the same particular target sequence. The mismatch may comprise one or more bases. While the mismatch(es) may be located anywhere in the mismatch probe, terminal mismatches are less desirable because a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

A homo-mismatch substitutes an adenine (A) for a thymine (T) and vice versa and a guanine (G) for a cytosine (C) and vice versa. For example, if the target sequence was: AGGTCCA, a probe designed with a single homo-mismatch at the central, or fourth position, would result in the following sequence: TCCTGGT.

In one embodiment, pairs are present in perfect match and mismatch pairs, one probe in each pair being a perfect match to the target sequence and the other probe being identical to the perfect match probe except that the central base is a homo-mismatch. Mismatch probes provide a control for non-specific binding or cross-hybridization to a nucleic acid in the sample other than the target to which the probe is directed. Thus, mismatch probes indicate whether hybridization is or is not specific. For example, if the target is present, the perfect match probes should be consistently brighter than the mismatch probes because fluorescence intensity, or brightness, corresponds to binding affinity. See e.g., U.S. Pat. No. 5,324,633. Finally, the difference in intensity between the perfect match and the mismatch probe (I(PM)−I(MM)) provides a good measure of the concentration of the hybridized material. See WO 98/11223.

Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

Nucleic acid or nucleic acid sequence, as used herein, refers broadly to a deoxyribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

Oligonucleotide or polynucleotide as used herein refers broadly to a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). See U.S. Pat. No. 6,156,501. The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure.

Patient, as used herein, refers broadly to any animal who is in need of treatment either to alleviate a disease state or to prevent the occurrence or reoccurrence of a disease state. Also, “Patient” as used herein, refers broadly to any animal who has risk factors, a history of disease, susceptibility, symptoms, signs, was previously diagnosed, is at risk for, or is a member of a patient population for a disease. The patient may be a clinical patient such as a human or a veterinary patient such as a companion, domesticated, livestock, exotic, or zoo animal. The term “subject” may be used interchangeably with the term “patient”.

Probe as used herein refers broadly to a nucleic acid capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, a linkage other than a phosphodiester bond may join the bases in probes, so long as it does not interfere with hybridization. Thus, probes may be peptide nucleic acids in which the constituent bases are joined by peptide bonds rather than phosphodiester linkages.

Real Time RT-PCR as used herein refers broadly to a method or device used therein that allows for the simultaneous amplification and quantification of specific RNA transcripts in a sample.

Specifically bind or specifically binds as used herein refers broadly to an antibody that preferentially binds to a particular antigenic epitope, but does not necessarily bind only to that particular antigenic epitope.

Solid support, support, and substrate as used herein, are used interchangeably, and refer broadly to a material or group of materials having a rigid or semi-rigid surface or surfaces. In one embodiment, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See, e.g., U.S. Pat. No. 5,744,305 for exemplary substrates.

Substantially similar as used herein refers broadly to the levels of expression of individual genes are preferably within the range of +/−1-5 fold of the level of expression by a normal cumulus cell, more preferably within the range of +/−1-3-fold, still more preferably within the range of +1-1-1.5 fold and most preferably within the range of +/−1.0-1.3, 1.0-1.2 or 1.0-1.2 fold of the detected levels of expression of the gene or polypeptide by a normal cumulus cell.

Synthetic antibody as used herein refers broadly to an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Target as used herein refers broadly to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, oligonucleotides, nucleic acids, antibodies, cell membrane receptors, monoclonal antibodies and antisera reactive with specific antigenic determinants (e.g., on viruses, cells or other materials), drugs, peptides, cofactors, lectins, sugars, polysaccharides, cells, cellular membranes, and organelles. Targets are sometimes referred to in the art as anti-probes. As the term targets is used herein, no difference in meaning is intended.

Zona pellucida as used herein refers broadly to the outermost region of an oocyte.

### Non-Invasive Methods of Assessing Oocyte Competency

The methods described herein utilize the cumulus cells to derive information about the genetic and chromosomal status of the oocyte for implantation and fertilization purposes.

In the ovary, human oocytes are surrounded by a cloud of tiny cells, known as cumulus cells. The oocytes and the cumulus cells are in constant communication and depend upon each other for continued viability. The inventors surprisingly discovered that the presence of chromosome abnormalities, which occur in human oocytes and are incompatible with the formation of healthy embryos, affect the surrounding cumulus cells. For several decades known chromosome problems are common in human oocytes and are the major cause of miscarriage, and are responsible for conditions such as Trisomy 21.

Thus, chromosome abnormalities in the oocyte were found to result in changes in the surrounding cumulus cells, allowing for testing of the oocytes, before they are fertilized, revealing those with the correct number of chromosomes as well as those that are abnormal. These results are unexpected because cumulus cells (CCs) are routinely stripped off oocytes during IVF treatments and are usually discarded.

The inventors examined the polar bodies and cumulus cells from 26 oocytes donated by women undergoing pre-implantation genetic screening (PGS). The researchers identified a total of 13 normal and 13 abnormal oocytes by testing the polar bodies. The inventors examined the active individual genes in the cumulus cells that had surrounded each oocyte using two different methods. First, using a microarray it was surprisingly found that 729 genes were expressed differently in cumulus cells that surrounded oocytes that contained an incorrect number of chromosomes. In particular, 14 genes appeared to have highly significant differences in activity when their corresponding oocyte was abnormal. The inventors also analyzed that 95 of the 729 genes, including the 14 very significant genes using as real-time polymerase chain reaction (PCR). The real-time PCR confirmed that most of the genes highlighted by the microarray showed altered activity in cumulus cells associated with abnormal oocytes. Several of these genes are involved in vital cellular functions of the cumulus cells and oocyte they surround, such as cell signaling and regulation, hormonal response, and cell death. The present invention has the advantage of avoiding the fertilization of abnormal oocytes, which might have some ethical advantages over the current invasive methods. Also the present invention is faster and less expensive than current methods. In addition, current diagnostic methods available for preimplantation genetic screening only provide information on the chromosome status of an oocyte. Although this is an important aspect of oocyte quality, it is not the only factor influencing the ability of the oocyte to lead to a successful pregnancy. The extra genetic information available from examining the cumulus cells may provide more detailed evaluation of an oocyte's potential to lead to a successful pregnancy and a healthy live birth.

The method involves detecting the levels of expression of at least one nucleic acid that encodes SPSB2 and TP53I3 or polypeptide encoded thereby and optionally another gene or polypeptide listed in Tables 2 and 3 or the 14 genes selected from the group consisting of B3GALNT2, C22orf29, CCL16, DCBLD1, DCC1, DHX9, OTUD5, RBBP6, SEPT11, SLC25A36, TACSTD2, Unassigned (helicase), (Table 5) that are expressed at characteristic levels by cumulus cells associated with (surrounding) oocytes and are correlated with the competence of a mammalian oocyte for fertilization and/or implantation.

As described herein the inventors have determined as set of genes expressed in cumulus cells that are biomarkers for with the competence of a mammalian oocyte for fertilization and/or implantation. They demonstrated that genes expression profile of cumulus cells which surrounds oocyte correlated to different competence of a mammalian oocyte, allowing the identification of a oocytes suitable for fertilization and/or implantation (e.g., oocytes with normal karyotype, absent any chromosomal abnormalities, e.g., aneuploidy). The methods described herein comprise the analysis of cumulus cells surrounding the oocyte as a rapid, non-invasive approach for oocyte selection.

The set of predictive genes in Tables 2 and 3 and the 14 gene set identified in Table 5 are known human genes. However, the expression of these genes (on cumulus cells) had not heretofore been correlated to oocyte competency or embryo development. Therefore, this invention relates to a method for selecting a competent oocyte, comprising a step of measuring the expression level of specific genes in a cumulus cell surrounding said oocyte, wherein said genes include at least one nucleic acid that encodes SPSB2 and TP53I3 or polypeptide encoded thereby and optionally another at least one of the genes listed in Table 1 and 2 or the 14 genes selected from the group consisting of B3GALNT2, C22orf29, CCL16, DCBLD1, DCC1, DHX9, OTUD5, RBBP6, SEPT11, SLC25A36, TACSTD2, Unassigned (helicase).

The methods of the invention may further comprise a step consisting of comparing the expression level of the genes in the sample with a control, wherein detecting differential in the expression level of the genes between the sample and the control is indicative whether the oocyte is competent. The control may consist in sample comprising cumulus cells associated with a competent oocyte or in a sample comprising cumulus cells associated with an unfertilized oocyte.

The method of evaluating the competence of a mammalian oocyte for implantation described herein may comprise determining the level of marker expression of determining the level of marker expression of at least one nucleic acid that encodes SPSB2 and TP53I3 or polypeptide encoded thereby and optionally another at least one gene selected from the group consisting of B3GALNT2, C22orf29, CCL16, DCBLD1, DCC1, DHX9, OTUD5, RBBP6, SEPT11, SLC25A36, TACSTD2, Unassigned (helicase), in a sample; and comparing the level of marker expression in the sample with a control or reference standard, wherein detecting differential marker expression between the sample and the control is indicative of the competence of the oocyte for implantation.

The methods of evaluating the competence of a mammalian oocyte for fertilization may comprise determining the level of marker expression of determining the level of marker expression of at least one nucleic acid that encodes SPSB2 and TP53I3 or polypeptide encoded thereby and optionally another at least one gene selected from the group consisting of B3GALNT2, C22orf29, CCL16, DCBLD1, DCC1, DHX9, OTUD5, RBBP6, SEPT11, SLC25A36, TACSTD2, Unassigned (helicase), in a sample; and comparing the level of marker expression in the sample with a control or reference standard, wherein detecting differential marker expression between the sample and the control is indicative of the competence of the oocyte for implantation (e.g., absence of chromosomal abnormalities). The sample for use in the methods described herein may be derived from a cumulus cell, an oocyte, and/or a polar body. Further, the sample may be a nucleic acid sample (e.g., cDNA, mRNA) or an amino acid sample (e.g., proteins).

The methods of the invention are applicable preferably to human women but may be applicable to other mammals (e.g., primates, dogs, cats, pigs, cattle, sheep, goats, llamas).

### Cumulus Cells

Cumulus cells (CCs) are specialized granulosa cells surrounding and nourishing the oocyte. These cells surround the fully-grown oocyte to form a cumulus-oocyte complex. Cumulus cells provide key products for the acquisition of developmental competence and differ from granulosa cells in their hormonal responses and growth factors they produce. The absence or insufficient numbers of cumulus cells impairs embryo production. For example, denuded oocytes in culture cannot undergo normal fertilization and development. Cumulus cells are required for the successful maturation of oocytes and also for fertilization. These cells synthesize an abundant mucoelastic extracellular matrix, which promotes oocyte extrusion from the follicle, a 20-40 fold increase in the volume of the cumulus mass, and probably also functions as a selective barrier for sperm. Salustri (2000) Int J Dev Biol. 44(6): 591-7. For cellular and molecular events during oocyte maturation and the formation of the extracellular matrix of the cumulus-oocyte complex. See also Russell and Salustry (2006) Semin Reprod Med. 24(4): 217-27 and Kimura, et al. (2007) Soc Reprod Fertil Suppl. 63: 327-42.

Cumulus cells show high expression of many enzymes of the glycolytic pathway and also neutral amino acid transporters. Their expression is promoted by paracrine factors secreted by oocytes [Eppig, et al. (2005) Biol Reprod. 73(2): 351-7 & Sugiura, et al. (2005) Dev Biol. 279(1): 20-301, which themselves are unable to take up L-alanine and poorly metabolize glucose for energy production and thus depend on cumulus cells for their provision. Biggers, et al. (1967) Proc Natl Acad Sci USA 58(2): 560-7; Colonna & Mangia (1983) Biol Reprod. 28(4): 797-803; Donahue and Stern (1968) J Reprod Fertil. 17(2): 395-8; Eppig, et al. (2005) Biol Reprod. 73(2): 351-7; Haghighat & Van Winkle (1990) J Exp Zool. 253(1): 71-82; Leese & Barton (1984) J Reprod Fertil. 72(1): 9-13; Leese & Barton J Exp Zool. (1985) 234(2): 231-6. Cumulus cell metabolic pathways, particularly glycolysis and cholesterol biosynthesis (Su, et al. (2008) Development 135(1): 111-21), are highly affected by mutations in two key protein growth factors derived from oocytes, BMP15 and GDF9. Juengel & McNatty (2005) Hum Reprod Update 11(2): 143-160.

The inventors investigated the origin of meiotic errors in oogenesis and identified novel molecular markers of aneuploidy that may be utilized for non-invasive screening, including in cumulus cells. Gene expression has a fundamental role in the regulation of virtually every aspect of cellular life. Analysis of gene expression can therefore give an indication of the various processes occurring within a cell, and may reveal the basis of biological problems, and provide information concerning viability. Wells, et al. (2005) Hum Reprod 20: 1339-1348. The fact that cumulus cells share the same follicular environment as the oocyte and are in close communication with it via gap junctions, thus the analysis of cumulus cells transcriptional activity may reveal information about the viability of the oocyte with which they are associated.

Moreover, cumulus cells can easily be collected without compromising the oocyte, allowing for non-invasive assays of oocyte competence by examining cumulus cells. The presence of a chromosome error in the oocyte may affect the levels of gene expression and protein production in the surrounding cumulus cells. Further, an inappropriate follicular environment could predispose to chromosome errors in the oocyte. This altered environment leaves a characteristic transcriptional footprint in the cumulus cells. The inventors surprisingly discovered that novel non-invasive markers of aneuploidy and general oocyte physiology and competence may be found in cumulus cells.

### Methods of Measuring Oocyte Competence

The present invention provides a method of distinguishing oocytes and embryos more likely to experience successful fertilization and implantation from oocytes and embryos less likely to experience successful fertilization and implantation by the analysis of marker expression in cumulus cells. In one embodiment, the method is non-invasive and the oocytes or embryos identified as more likely to experience successful fertilization and implantation remain viable for implantation. In one embodiment, the method is non-damaging and the oocytes or embryos identified as more likely to experience successful fertilization and implantation remain viable for implantation.

Oogenesis is a complex process that starts during early fetal development and oocytes arrest at prophase of meiosis I. Oocytes resume development at puberty in response to gonadotropic hormones and one oocyte is ovulated each month. The monthly cycles continue until menopause and oocytes provide foundations for early preimplantation development. Further, Oocyte mRNA and proteins support embryo until genome activation

For oocyte selection in the IVF lab, the object is to select a competent oocyte that is able to mature—cytoplasm and nucleus; able to undergo fertilization; able to support embryonic development; and able to lead to a successful pregnancy and birth. Typical oocyte selection criteria in IVF labs is done based on morphological assessment but there are problems with this approach including that oocyte competency cannot be predicted by morphology and aneuploidy cannot be screened through morphology.

Aneuploidy is frequent in humans, leading to high mental retardation and miscarriage rates. Most aneuploidies arise during oogenesis and there is a close relationship between female age and oocyte aneuploidy.

Preimplantation Genetic Screening includes the cytogenetic analysis of polar bodies, blastomeres, trophectoderm (TE) samples. Although these screening methods are effective, the invasive nature of biopsy might impact embryo viability. Fragouli, et al. (2006) Hum reprod 21: 2319-2328; Goossens, et al. (2008) Hum Reprod. 23(12): 2629-45; Fragouli, et al. (2010) Fertil. Steril 94: 875-887. Accordingly, the methods described herein provide a non-invasive assessment of oocytes or embryos with information on chromosome ploidy, competence, and implantation potential.

The inventors surprisingly discovered that cumulus cells may be used in pre-implantation genetic screening to assess chromosome ploidy, competence, and implantation potential of oocytes which they surround. The inventors found that events happening at pre-ovulatory stage in cumulus-oocyte complex of critical importance for maturation, fertilization and embryo competence. The cumulus cells are generated by granulosa cells and the cumulus cell layers surround oocyte in antral follicles. The bi-directional communication between oocyte and cumulus cells via gap junctions allows for a continuous exchange of proteins and metabolites. The inventors discovered that oocyte aneuploidy affects cumulus cell gene expression and that cumulus cells may be used for the non-invasive assessment of oocyte aneuploidy and general quality.

The inventors used arrays and real-time PCR to conduct a comprehensive analysis of cumulus cell transcriptome allowing for an insight into follicular microenvironment of aneuploid oocytes. Aneuploid oocytes are associated with transcriptionally quiescent and less proliferative cumulative cells. Further, the abnormal expression of genes regulating metabolism, cell-cell communication, hypoxia, and apoptosis was found. Thus there is a relationship between follicular microenvironment and oocyte aneuploidy. In particular, fourteen genes are useful targets for non-invasive test development. For example, the SPSB2 gene (e.g., UniProtKB Q99619) expression in cumulus cells correlated with oocyte chromosome status and potential to lead to live birth.

For example, the following biological processes affected in cumulus cells of aneuploid oocytes (with corresponding genes) in Table 1.

TABLE 1
Biological Process Affected In Cumulus Cells of aneuploid oocytes with
corresponding genes that are differentially expressed

| Biological Process | Gene |
|---|---|
| Cell Adhesion | DCBLD1 |
| Cell communication | DCBLD1; SPSB2; CCL16; TACSDT2 |
| Cell cycle | OTUD5; DCCl; Septin 11 |
| Cellular processes | OTUD5; DCLBD1; SPSB2; CCL16; |
| | TACSDT2; DCC1; C22orf29; Unassigned |
| | (Helicase) |
| Developmental processes | OTUD5; DCLBD1 |
| Homeostatic processes | CCL16 |
| Immune system processes | DCLBD1; CCL16 |
| Metabolic processes | B3GALNT2; DCBLD1; SLC25A36; |
| | RBBP6; DSCC1; DHX9; Unassigned |
| | (Helicase) |
| Response to stimulus | DCBLD1; CCL16 |
| System processes | DCBLD1 |
| Transport | DCBLD1; SLC25A36 |

In one embodiment, the assessment of marker expression in oocytes, cumulus cells, follicular fluid, or culture medium is used to assess the competence of an oocyte for implantation. The assessment may be performed before implantation, to assist in maximizing the implantation of chromosomally normal embryos or to assist in minimizing the implantation of chromosomally abnormal embryos.

In one embodiment, the assessment of marker expression in oocytes, cumulus cells, follicular fluid, or culture medium is used to assess the competence of an oocyte for fertilization. The assessment may be performed before fertilization, to assist in maximizing the generation of chromosomally normal embryos or to assist in minimizing the generation of chromosomally abnormal embryos.

In one embodiment, the assessment of marker expression in oocytes, cumulus cells, follicular fluid, or culture medium is used to assess the quality of an oocyte for fertilization, implantation or long-term storage for later use by, for example, freezing.

In one embodiment, the products of differentially expressed markers are used for in vitro assessment of oocyte aneuploidy. In one embodiment, markers, gene products, RNA, proteins, and metabolites are assessed in follicular fluid, cumulus cells, polar bodies, oocytes, embryos or culture media in which the oocytes, cumulus cells, or embryos are cultured.

In one aspect, the markers displaying differential expression are used to diagnose chromosome abnormality. The assessment of marker expression in oocytes or cumulus cells is used to optimize methods for ovarian stimulation. The assessment of marker expression in oocytes or cumulus cells is also used to modify or optimize an in vitro maturation medium. Further, the assessment of marker expression in oocytes or cumulus cells is used to assay the effects of toxicants on human oocytes.

Preferably, the oocytes, cumulus cells, and embryos are human. However, the oocytes, cumulus cells, and embryos may be obtained from other non-human animals, preferably, domesticated animals including but not limited to livestock (e.g., cows, cattle, horses, camels, pigs, goats, sheep, llamas).

The assessment of marker expression in oocytes or cumulus cells may be used to assist the proper function of affected gene expression pathways by modifying the levels of components in culture media to, for example, optimize ovarian stimulation.

The assessment of marker expression in oocytes or cumulus cells may be used to guide the design of culture media, which supports proper chromosome segregation and minimizes chromosome/chromatid imbalance to, for example, optimize ovarian stimulation.

The assessment of marker expression in oocytes or cumulus cells may be used, for example, to guide the design of dietary supplements, to reduce the chance of abnormal oocytes being formed, to improve fertility, to increase the number of years that a female remains fertile, and to reduce the risk of chromosomal conditions such as, for example, Down syndrome (Trisomy 21).

The invention contemplates the use of methods for the identification of differentially expressed markers of chromosome normality and abnormality and differentially expressed markers of oocyte competence and incompetence, as well as methods for the detection of the expression products of differentially expressed markers of chromosome normality and abnormality and differentially expressed markers of oocyte competence and incompetence.

The invention contemplates the identification of differentially expressed markers by whole genome nucleic acid microarray, to identify markers differentially expressed between oocytes competent for implantation and oocytes not competent for implantation. The invention further contemplates using methods known to those skilled in the art to detect and to measure the level of differentially expressed marker expression products, such as RNA and protein, to measure the level of one or more differentially expressed marker expression products in an oocyte, as well as follicular fluid, cumulus cells, and culture medium associated with an oocyte, to evaluate the chromosomal and genetic competence of the oocyte and its potential for implantation.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Sambrook, et al. (2001) Molecular Cloning: A Laboratory Manual (2^{nd} Ed.) Cold Spring Harbor Laboratory Press); Stryer (1995) Biochemistry (4^{th} Ed.) Freeman; Herdewijn (2005) Oligonucleotide Synthesis Methods and Applications Humana Press, Inc.; Nelson & Cox (2008) Lehninger Principles of Biochemistry (5^{th} Ed) W.H. Freeman; and Berg, et al. (2006) Biochemistry (6^{th} Ed.) W.H. Freeman.

Nucleic acid arrays that are useful in the present invention include arrays such as those commercially available from Affymetrix (Santa Clara, Calif.), and from Applied Biosystems (Foster City, Calif.), and from Agilent Technologies (Santa Clara, Calif.).

The present invention also contemplates sample preparation methods in certain embodiments. Prior to or concurrent with marker expression analysis, the expression product sample may be amplified using a variety of mechanisms, some of which may employ PCR. See, for example, Erlich (Ed.) (1994) PCR Technology: Principles and Applications for DNA Amplification Freeman Press; Bartlett & Stirling (Ed.) (2003) PCR Protocols Humana Press; Mattila, et al. (1991) Nucleic Acids Res. 19: 4967; Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675.

Other suitable amplification methods include the ligase chain reaction (LCR) (for example, Wu & Wallace (1989) Genomics 4: 560, Landegren, et al. (1988) Science 241: 1077 and Barringer, et al. (1990) Gene 89: 117), transcription amplification (Kwoh, et al. (1989) Proc. Natl. Acad. Sci. USA 86: 1173 and WO 88/10315), self-sustained sequence replication (Guatelli, et al. (1990) Proc. Nat. Acad. Sci. USA 87: 1874 and WO 90/06995), selective amplification of target polynucleotide sequences (U.S. Pat. No. 6,410,276), consensus sequence primed PCR (CP-PCR) (U.S. Pat. No. 4,437,975), arbitrarily primed PCR (AP-PCR) (U.S. Pat. Nos. 5,413,909 and 5,861,245), degenerate oligonucleotide primed PCR (DOP-PCR) (Wells, et al. (1999) Nuc Acids Res 27: 1214-1218) and nucleic acid based sequence amplification (NABSA). See U.S. Pat. Nos. 5,409,818; 5,554,517; and 6,063,603. Other amplification methods that may be used are described in, U.S. Pat. Nos. 5,242,794, 5,494,810, 4,988,617; and 6,582,938.

Additional methods of sample preparation and techniques for reducing the complexity of a nucleic sample are described in Dong, et al. (2001) Genome Research 11: 1418, in U.S. Pat. Nos. 6,361,947, 6,391,592; and 6,872,529, U.S. Patent Application Publication 2003/0096235 and 2003/0082543.

Methods for conducting polynucleotide hybridization assays, for example, but not limited to northern blots, southern blots, and nucleic acid microarrays, have been developed in the art. Hybridization assay procedures and conditions will vary depending on the application and are selected in accordance with the general binding methods known including those referred to in: Sambrook, et al. (2001) Molecular Cloning: A Laboratory Manual (2^{nd} Ed.) Cold Spring Harbor Laboratory Press; Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625; 7,689,022; and WO 99/47964.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; 6,225,625; 7,689,022; 7,317,415; and WO 99/47964.

The practice of the present invention may also employ software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2.sup.nd ed., 2001). See U.S. Pat. No. 6,420,108.

The present invention may also make use of various computer program products and software for a variety of purposes, such as probe design, management of data, analysis, and instrument operation. See U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Additionally, the present invention may have preferred embodiments that include methods for providing genetic information over networks such as the Internet as shown in U.S. Patent Application Publication Nos. 2003/0100995; 2003/0120432; 2004/0002818; 2004/0126840; 2004/0049354; 2003/0097222; 20020183936.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### Methods of Detection

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

A variety of immunoassay formats, including competitive and non-competitive immunoassay formats, antigen capture assays, enzyme immunoassay (EIA), radioimmunoassay (RIA), counting immunoassay (CIA), immunoenzymoetric assay (IEMA), chemiluminescent immunoassay (CLIA), ELISA, LIA (luminescent immunoassay), fluorescent immunoassay, two-antibody sandwich assays, and three-antibody sandwich assays are useful methods of the invention. Self, et al. (1996) Curr. Opin. Biotechnol. 7: 60-65 and David Wild [Ed.] (2008) The Immunoassay Handbook (3^{rd} Ed.) The invention should not be construed to be limited to any one type of immunoassay.

In one embodiment, the method of the invention relies on one or more antigen capture assays. In one such antigen capture assay, antibody is bound to a solid support, and sample is added such that antigen is bound by the antibody. After unbound proteins are removed by washing, the amount of bound antigen can be quantified, if desired, using, for example, but not limited to, a radioassay. Harlow & Lane (1999) Using Antibodies: A Laboratory Manual Cold Spring Harbor Laboratory Press.

Enzyme-linked immunosorbent assays (ELISAs) are useful in the methods of the invention. An enzyme such as, but not limited to, horseradish peroxidase (HRP), alkaline phosphatase (AP), beta-galactosidase or urease can be linked, for example, to an antigen antibody or to a secondary antibody for use in a method of the invention. A horseradish-peroxidase detection system may be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which may be used with the chromogenic substrate p-nitrophenyl phosphate to yield a soluble product readily detectable at 405 nm. Similarly, a beta-galactosidase detection system may be used with the chromogenic substrate o-nitrophenyl-beta-D-galactopyranoside (ONPG) to yield a soluble product detectable at 410 nm. Alternatively, a urease detection system may be used with a substrate such as urea-bromocresol purple (Sigma Immunochemicals, St. Louis, Mo.) Useful enzyme-linked primary and secondary antibodies can be obtained from any number of commercial sources.

Chemiluminescent detection is also useful for detecting antigen or for determining a quantity of antigen according to a method of the invention. Chemiluminescent secondary antibodies may be obtained from any number of commercial sources. Fluorescent detection is also useful for detecting antigen or for determining a level of antigen in a method of the invention. Useful fluorochromes include, but are not limited to, DAPI, fluorescein, Hoechst 33258, R-phycocyanin, B-phycoerythrin, R-phycoerythrin, rhodamine, Texas red and lissamine-Fluorescein- or rhodamine-labeled antigen-specific antibodies.

Radioimmunoassays (RIAs) are also useful in the methods of the invention. Such assays are well known in the art, and are described for example in Brophy, et al. (1990) Biochem. Biophys. Res. Comm. 167: 898-903 and Guechot, et al. (1996) Clin. Chem. 42: 558-563 and Surhone, et al. (Eds.) Radioimmunassay (2010) VDM Verlag. Radioimmunoassays are performed, for example, using Iodine-125-labeled primary or secondary antibody. Harlow & Lane (1999) Using Antibodies: A Laboratory Manual Cold Spring Harbor Laboratory Press.

A signal emitted from a detectable antibody is analyzed, for example, using a spectrophotometer to detect color from a chromogenic substrate; a radiation counter to detect radiation, such as a gamma counter for detection of Iodine-125; or a fluorometer to detect fluorescence in the presence of light of a certain wavelength. Where an enzyme-linked assay is used, quantitative analysis of the amount of antigen is performed using a spectrophotometer. It is understood that the assays of the invention can be performed manually or, if desired, can be automated and that the signal emitted from multiple samples can be detected simultaneously in many systems available commercially.

The methods of the invention also encompass the use of capillary electrophoresis based immunoassays (CEIA), which can be automated, if desired. Immunoassays also may be used in conjunction with laser-induced fluorescence as described, for example, in Schmalzing, et al. (1997) Electrophoresis 18: 2184-2193 and Bao (1997) J. Chromatogr. B. Biomed. Sci. 699: 463-480. Liposome immunoassays, such as flow-injection liposome immunoassays and liposome immunosensors, may also be used to detect antigen according to the methods of the invention. See Rongen, et al. (1997) J. Immunol. Methods 204: 105-133. See also Walid (Ed.) The Immunoassay Handbook (3^{rd} Ed.) (2005) Elsevier Ltd.

Sandwich enzyme immunoassays may also be useful in the methods of the invention. In a two-antibody sandwich assay, a first antibody is bound to a solid support, and the antigen is allowed to bind to the first antibody. The amount of antigen is quantified by detecting and measuring the amount of a detectable second antibody that binds to the complex of the antigen and the first antibody. In a three-antibody sandwich assay, a first antibody is bound to a solid support, and the antigen is allowed to bind to the first antibody. Then a second antibody is added and is allowed to bind to the antigen, which is bound to the first antibody. The amount of antigen is quantified by detecting and measuring the amount of a detectable third antibody that binds to the second antibody.

Quantitative western blotting may also be used to detect antigen or to determine a level of antigen in a method of the invention. Western blots are quantified using well known methods such as scanning densitometry. Parra, et al. (1998) J. Vasc. Surg. 28: 669-675. Fluorescence activated cell sorting (FACS) analysis may also be used to detect antigen or to determine the level of antigen in a method of the invention. Using FACS analysis, cells may be stained with one or more fluorescent dyes specific to cell components of interest, and fluorescence of each cell is measured as it rapidly transverses the excitation beam (laser or mercury arc lamp).

Fluorescence provides a quantitative measure of various biochemical and biophysical properties of the cell, as well as a basis for cell sorting. Other measurable optical parameters include light absorption and light scattering, the latter being applicable to the measurement of cell size, shape, density, granularity, and stain uptake. See Darzynkiewicz, et al. (2004) Cytometry: New Developments (4^{th} Ed.) Elsevier Academic Press.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound protein in a liquid phase from a solid phase support to which antigen-antibody complexes are bound. Solid supports which can be used in the practice of the invention include substrates such as nitrocellulose (e.g., in membrane or microtitre well form); polyvinylchloride (e.g., sheets or microtitre wells); polystyrene latex (e.g., beads or microtitre plates); polyvinylidine fluoride; diazotized paper; nylon membranes; activated beads, magnetically responsive beads, and the like. More particularly, an ELISA method can be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

Alternatively an immunohistochemistry (IHC) method may be preferred. IHC specifically provides a method of detecting targets in a sample or tissue specimen in situ. The overall cellular integrity of the sample is maintained in IHC, thus allowing detection of both the presence and location of the targets of interest. Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision® (DakoCytomation), Powervision® (Immunovision, Springdale, Ariz.), the NBA® kit (Zymed Laboratories Inc., South San Francisco, Calif.), HistoFine® (Nichirei Corp, Tokyo, Japan).

In particular embodiment, a tissue section (e.g. a sample comprising cumulus cells) may be mounted on a slide or other support after incubation with antibodies directed against the proteins encoded by the genes of interest. Then, microscopic inspections in the sample mounted on a suitable solid support may be performed. For the production of photomicrographs, sections comprising samples may be mounted on a glass slide or other planar support, to highlight by selective staining the presence of the proteins of interest.

Therefore IHC samples may include, for instance: (a) preparations comprising cumulus cells (b) fixed and embedded said cells and (c) detecting the proteins of interest in said cells samples. In some embodiments, an IHC staining procedure may comprise steps such as: cutting and trimming tissue, fixation, dehydration, paraffin infiltration, cutting in thin sections, mounting onto glass slides, baking, deparaffination, rehydration, antigen retrieval, blocking steps, applying primary antibodies, washing, applying secondary antibodies (optionally coupled to a suitable detectable label), washing, counter staining, and microscopic examination.

### Nucleic Acid Amplification

Assays for amplification of the known sequence are also disclosed. For example primers for long range PCR may be designed to amplify regions of the sequence. For RNA, a first reverse transcriptase step may be used to generate double stranded DNA from the single stranded RNA. The array may be designed to detect sequences from an entire genome; or one or more regions of a genome, for example, selected regions of a genome such as those coding for a protein or RNA of interest; or a conserved region from multiple genomes; or multiple genomes, Arrays and methods of genetic analysis using arrays is described in Cutler, et al. (2001) Genome Res. 11(11): 1913-1925 and Warrington, et al. (2002) Hum Mutat 19: 402-409 and in U.S. Patent Application Publication No 2003/0124539.

Hybridization probes are oligonucleotides capable of binding in a base-specific manner to a complementary strand of nucleic acid. Such probes include peptide nucleic acids, as described in Nielsen, et al. (1991) Science 254: 1497-1500, and other nucleic acid analogs and nucleic acid mimetics. See U.S. Pat. No. 6,156,501.

The term hybridization refers to the process in which two single-stranded nucleic acids bind non-covalently to form a double-stranded nucleic acid; triple-stranded hybridization is also theoretically possible. Complementary sequences in the nucleic acids pair with each other to form a double helix. The resulting double-stranded nucleic acid is a “hybrid.” Hybridization may be between, for example to complementary or partially complementary sequences. The hybrid may have double-stranded regions and single stranded regions. The hybrid may be, for example, DNA:DNA, RNA:DNA or DNA:RNA. Hybrids may also be formed between modified nucleic acids. One or both of the nucleic acids may be immobilized on a solid support. Hybridization techniques may be used to detect and isolate specific sequences, measure homology, or define other characteristics of one or both strands.

The stability of a hybrid depends on a variety of factors including the length of complementarity, the presence of mismatches within the complementary region, the temperature and the concentration of salt in the reaction. Hybridizations are usually performed under stringent conditions, for example, at a salt concentration of no more than 1 M and a temperature of at Least 25° C. For example, conditions of 5×SPE (750 mM NaCl, 50 mM NaPhosphate, 5 mM EDTA, pH 7.4) or 100 mM MES, 1 M Na, 20 mM EDTA, 0.01% Tween-20 and a temperature of 25-50° C. are suitable for allele-specific probe hybridizations. In a particularly preferred embodiment, hybridizations are performed at 40-50° C. Acetylated BSA and herring sperm DNA may be added to hybridization reactions. Exemplary conditions for hybridization include low stringency, medium stringency, high stringency, or very high stringency conditions, which, as used herein, refers broadly to conditions for nucleic acid hybridization and washing. Guidance for performing hybridization reactions can be found in Ausubel, et al. (2002) Short Protocols in Molecular Biology (5^{th} Ed.) John Wiley & Sons, NY. Exemplary specific hybridization conditions include but are not limited to: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and (4) very high stringency hybridization conditions are 0.5M sodium phosphate, 7% SDS at 65° C., followed by one or more washes at 0.2×SSC, 1% SDS at 65° C.

### Labels

In one embodiment, the hybridized nucleic acids are detected by detecting one or more labels attached to the sample nucleic acids. The labels may be incorporated by any of a number of means well known to those of skill in the art. In one embodiment, the label is simultaneously incorporated during the amplification step in the preparation of the sample nucleic acids. Thus, for example, PCR with labeled primers or labeled nucleotides will provide a labeled amplification product. In another embodiment, transcription amplification, as described above, using a labeled nucleotide (e.g., fluorescein-labeled UTP and/or CTP) incorporates a label into the transcribed nucleic acids. In another embodiment PCR amplification products are fragmented and labeled by terminal deoxytransferase and labeled dNTPs. Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g., with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase.

As stated above, nucleic acid and nucleic acid probes may be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

The nucleic acid and nucleic acid probes described herein may be modified post-translationally to add effector moieties such as chemical linkers, detectable moieties such as for example fluorescent dyes, enzymes, substrates, bioluminescent materials, radioactive materials, chemiluminescent moieties, a cytotoxic agent, radioactive materials, or functional moieties.

A wide variety of entities, e.g., ligands, may be coupled to the oligonucleotides as known in the art. Ligands may include naturally occurring molecules, or recombinant or synthetic molecules. Exemplary ligands include, but are not limited to, avadin, biotin, peptides, peptidomimetics, polylysine (PLL), polyethylene glycol (PEG), mPEG, cationic groups, spermine, spermidine, polyamine, thyrotropin, melanotropin, lectin, glycoprotein, surfactant protein A, mucin, glycosylated polyaminoacids, transferrin, aptamer, immunoglobulins (e.g., antibodies), insulin, transferrin, albumin, sugar, lipophilic molecules (e.g., steroids, bile acids, cholesterol, cholic acid, and fatty acids), vitamin A, vitamin E, vitamin K, vitamin B, folic acid, B12, riboflavin, biotin, pyridoxal, vitamin cofactors, lipopolysaccharide, hormones and hormone receptors, lectins, carbohydrates, multivalent carbohydrates, radiolabeled markers, fluorescent dyes, and derivatives thereof. See, e.g., U.S. Pat. Nos. 6,153,737; 6,172,208; 6,300,319; 6,335,434; 6,335,437; 6,395,437; 6,444,806; 6,486,308; 6,525,031; 6,528,631; and 6,559, 279.

Additionally, moieties may be added to the antigen or epitope to increase half-life in vivo (e.g., by lengthening the time to clearance from the blood stream. Such techniques include, for example, adding PEG moieties (also termed pegilation), and are well-known in the art.

A nucleic acid and nucleic acid probes described herein may be “attached” to a substrate when it is associated with the solid label through a non-random chemical or physical interaction. The attachment may be through a covalent bond. However, attachments need not be covalent or permanent. Materials may be attached to a label through a “spacer molecule” or “linker group.” Such spacer molecules are molecules that have a first portion that attaches to the biological material and a second portion that attaches to the label. Thus, when attached to the label, the spacer molecule separates the label and the biological materials, but is attached to both. Methods of attaching biological material (e.g., label) to a label are well known in the art, and include but are not limited to chemical coupling.

### Detectable Labels

For example, detectable labels suitable for use in the present invention include any composition detectable by spectroscopic, photochemical, biochemical, immunochemical, electrical, optical or chemical means. The nucleic acid and nucleic acid probes described herein may be modified post-translationally to add effector labels such as chemical linkers, detectable labels such as for example fluorescent dyes, enzymes, substrates, bioluminescent materials, radioactive materials, and chemiluminescent labels, or functional labels such as for example streptavidin, avidin, biotin, a cytotoxin, a cytotoxic agent, and radioactive materials. Further exemplary enzymes include, but are not limited to, horseradish peroxidase, acetylcholinesterase, alkaline phosphatase, β-galactosidase and luciferase. Further exemplary fluorescent materials include, but are not limited to, rhodamine, fluorescein, fluorescein isothiocyanate, umbelliferone, dichlorotriazinylamine, phycoerythrin and dansyl chloride. Further exemplary chemiluminescent labels include, but are not limited to, luminol. Further exemplary bioluminescent materials include, but are not limited to, luciferin and aequorin. Further exemplary radioactive materials include, but are not limited to, hydrogen-3 (³H), bismuth-213 (²¹³Bs), carbon-14 (¹⁴C), carbon-¹¹C) chlorine-18 (Cl¹⁸), chromium-51, (⁵¹Cr), cobalt-57 (⁵⁷Co), cobalt-60 (⁶⁰Co), copper-64 (⁶⁴Cu), copper-67 (⁶⁷Cu), dysprosium-165 (¹⁶⁵Dy), erbium-169 (¹⁶⁹Er), fluorine-18 (¹⁸F), gallium-67 (⁶⁷Ga), gallium-68 (⁶⁸Ga), germanium-68 (⁶⁸Ge), holmium-166 (¹⁶⁶Ho), indium-111 (¹¹¹In), iodine-125 (¹²⁵I), iodine-123 (¹²⁴I), iodine-124 (¹²⁴I), iodine-131 (¹³¹I), iridium-192 (¹⁹²Ir), iron-59 (⁵⁹Fe), krypton-81 (⁸¹Kr), lead-212 (²¹²Pb), lutetium-177 (¹⁷⁷Lu), molybdenum-99 (⁹⁹Mo), nitrogen-13 (¹³N), oxygen-15 (¹⁵O), palladium-103 (¹⁰³Pd), phosphorus-32 (³²P), potassium-42 (⁴²K), rhenium-186 (¹⁸⁶Re), rhenium-188 (¹⁸⁸Re), rubidium-81 (⁸¹Rb), rubidium-82 (⁸²Rb), samarium-153 (¹⁵³Sm), selenium-75 (⁷⁵Se), sodium-24 (²⁴Na), strontium-82 (⁸²Sr), strontium-89 (⁸⁹Sr), sulfur 35 (³⁵S), technetium-99m (⁹⁹Tc), thallium-201 (²⁰¹Tl), tritium (³H), xenon-133 (¹³³Xe), ytterbium-169 (¹⁶⁹Yb), ytterbium-177 (¹⁷⁷Yb), and yttrium-90 (⁹⁰Y). Further exemplary labels include but are not limited to biotin for staining with labeled streptavidin conjugate; anti-biotin antibodies, magnetic beads (e.g., Dynabeads®); fluorescent dyes (e.g., fluorescein, texas red, rhodamine, green fluorescent protein); phosphorescent labels; enzymes (e.g., horse radish peroxidase, alkaline phosphatase and others commonly used in an ELISA); and colorimetric labels such as colloidal gold or colored glass or plastic (e.g., polystyrene, polypropylene, latex) beads

Methods are known in the art for conjugating nucleic acids and nucleic acid probes described herein to a label, such as those methods described by Hermanson (2008) Bioconjugate Techniques [2^{nd} Ed.], Elsevier, Inc.; Rapley [Ed.] (2000) The Nucleic Acid Protocols Handbook Humana Press; and Symons (1989) Nucleic Acid Probes CRC Press, Inc. The use of such labels are also described, for example, in U.S. Pat. Nos. 3,817,837, 3,850,752, 3,939,350, 3,996,345, 4,277,437, 4,275,149 and 4,366,241.

### Substrates

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. See U.S. Pat. Nos. 5,770,358, 5,789,162, 5,708,153, 6,040,193 and 5,800,992.

Arrays may be packaged in such a manner as to allow for diagnostic use or can be an all-inclusive device; e.g., U.S. Pat. Nos. 5,856,174 and 5,922,591. Arrays are commercially available from, for example, Affymetrix (Santa Clara, Calif.) and Applied Biosystems (Foster City, Calif.), and are directed to a variety of purposes, including genotyping, diagnostics, mutation analysis, marker expression, and gene expression monitoring for a variety of eukaryotic and prokaryotic organisms. The number of probes on a solid support may be varied by changing the size of the individual features. In one embodiment the feature size is 20 by 25 microns square, in other embodiments features may be, for example, 8 by 8, 5 by 5 or 3 by 3 microns square, resulting in about 2,600,000, 6,600,000 or 18,000,000 individual probe features.

U.S. Pat. Nos. 5,800,992 and 6,040,138 describe methods for making arrays of nucleic acid probes that can be used to detect the presence of a nucleic acid containing a specific nucleotide sequence. Methods of forming high-density arrays of nucleic acids, peptides and other polymer sequences with a minimal number of synthetic steps are known. The nucleic acid array can be synthesized on a solid substrate by a variety of methods, including, but not limited to, light-directed chemical coupling, and mechanically directed coupling. For additional descriptions and methods relating to arrays. See U.S. Pat. Nos. 5,861,242, 6,027,880, 5,837,832, 6,723,503; 7,144,699; 7,300,788; WO 2003/060526; and U.S. Patent Application Publication No. 2005/0032074.

The nucleic acids and nucleic acid probes described herein may be attached to a substrate. A number of substrates (e.g., solid supports) known in the art are suitable for use with the nucleic acids and probes thereof described herein. The substrate may be modified to contain channels or other configurations. See Fung (2004) [Ed.] Protein Arrays: Methods and Protocols Humana Press and Kambhampati (2004) [Ed.] Protein Microarray Technology John Wiley & Sons.

Substrate materials include, but are not limited to acrylics, agarose, borosilicate glass, carbon (e.g., carbon nanofiber sheets or pellets), cellulose acetate, cellulose, ceramics, gels, glass (e.g., inorganic, controlled-pore, modified, soda-lime, or functionalized glass), latex, magnetic beads, membranes, metal, metalloids, nitrocellulose, NYLON®, optical fiber bundles, organic polymers, paper, plastics, polyacryloylmorpholide, poly(4-methylbutene), poly(ethylene terephthalate), poly(vinyl butyrate), polyacrylamide, polybutylene, polycarbonate, polyethylene, polyethyleneglycol terephthalate, polyformaldehyde, polymethacrylate, polymethylmethacrylate, polypropylene, polysaccharides, polystyrene, polyurethanes, polyvinylacetate, polyvinylchloride, polyvinylidene difluoride (PVDF), polyvinylpyrrolidinone, rayon, resins, rubbers, semiconductor materials, SEPHAROSE®, silica, silicon, styrene copolymers, TEFLON®, and variety of other polymers.

Substrates need not be flat and can include any type of shape including spherical shapes (e.g., beads) or cylindrical shapes (e.g., fibers). Materials attached to solid supports may be attached to any portion of the solid support (e.g., may be attached to an interior portion of a porous solid support material).

The substrate body may be in the form of a bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial. The substrate may be a singular discrete body (e.g., a single tube, a single bead), any number of a plurality of substrate bodies (e.g, a rack of 10 tubes, several beads), or combinations thereof (e.g., a tray comprises a plurality of microtiter plates, a column filled with beads, a microtiter plate filed with beads).

A nucleic acid or nucleic acid probe may be “attached” to a substrate when it is associated with the solid substrate through a non-random chemical or physical interaction. The attachment may be through a covalent bond. However, attachments need not be covalent or permanent. Materials may be attached to a substrate through a “spacer molecule” or “linker group.” Such spacer molecules are molecules that have a first portion that attaches to the biological material and a second portion that attaches to the substrate. Thus, when attached to the substrate, the spacer molecule separates the substrate and the biological materials, but is attached to both. Methods of attaching biological material (e.g., label) to a substrate are well known in the art, and include but are not limited to chemical coupling.

Plates, such as microtiter plates, which support and contain the solid-phase for solid-phase synthetic reactions may be used. Microtiter plates may house beads that are used as the solid-phase. By “particle” or “microparticle” or “nanoparticle” or “bead” or “microbead” or “microsphere” herein is meant microparticulate matter having any of a variety of shapes or sizes. The shape may be generally spherical but need not be spherical, being, for example, cylindrical or polyhedral. As will be appreciated by those in the art, the particles may comprise a wide variety of materials depending on their use, including, but not limited to, cross-linked starch, dextrans, cellulose, proteins, organic polymers including styrene polymers such as polystyrene and methylstyrene as well as other styrene copolymers, plastics, glass, ceramics, acrylic polymers, magnetically responsive materials, colloids, thoriasol, carbon graphite, titanium dioxide, nylon, latex, and TEFLON®. See e.g., “Microsphere Detection Guide” from Bangs Laboratories, Fishers, Ind.

The nucleic acids and nucleic acid probes may be attached to on any of the forms of substrates described herein (e.g., bead, box, column, cylinder, disc, dish (e.g., glass dish, PETRI dish), fiber, film, filter, microtiter plate (e.g., 96-well microtiter plate), multi-bladed stick, net, pellet, plate, ring, rod, roll, sheet, slide, stick, tray, tube, or vial). In particular, particles or beads may be a component of a gelling material or may be separate components such as latex beads made of a variety of synthetic plastics (e.g., polystyrene). The label (e.g., streptavidin) may be bound to a substrate (e.g., bead).

### Sequencing Methods

The invention also encompasses methods of sequencing of the substantially uniform nucleic acid fragments as prepared by the methods set forth herein. In one embodiment, the methods described herein may be used to prepare size fragments for paired end sequencing libraries of short insert (˜300 bases ˜100 nm pitch), long insert (˜3 kb ˜1 μm pitch), and ultralong insert (˜30 kb ˜10 μm pitch). The nucleic acid sequencing methods described herein can be automated.

Sequencing can be carried out using any suitable sequencing technique including, for example, sequencing by synthesis techniques wherein nucleotides are added successively to a free 3′ hydroxyl group, resulting in synthesis of a nucleic acid chain in the 5′ to 3′ direction. The nature of the nucleotide added is preferably determined after each nucleotide addition. Sequencing techniques using sequencing by ligation, wherein not every contiguous base is sequenced, and techniques such as massively parallel signature sequencing (MPSS) where bases are removed from, rather than added to the strands on the surface are also useful, as are techniques using detection of pyrophosphate release (pyrosequencing).

The initiation point for a sequencing reaction may be provided by annealing of a sequencing primer to a target nucleic acid present at a feature of an array. In this connection, a known adapter region that is present on a target nucleic acid, for example, a target nucleic acid from a cleavage reaction described previously herein, can be used as a priming site for annealing of a sequencing primer.

In a particular embodiment, a nucleic acid sequencing reaction can include steps of hybridising a sequencing primer to a single-stranded region of a linearised nucleic acid fragment (or amplification product thereof) that acts as a sequencing template, sequentially incorporating one or more nucleotides into a nucleic acid strand complementary to the region of the template strand to be sequenced, identifying the base present in one or more of the incorporated nucleotide(s) and thereby determining the sequence of a region of the template strand.

One preferred sequencing method which can be used in accordance with the invention relies on the use of modified nucleotides having removable 3′ blocks, for example, as described in WO 2004/018497 and U.S. Pat. No. 7,057,026. Once the modified nucleotide has been incorporated into the growing nucleic acid chain complementary to the region of the template being sequenced there is no free 3′-OH group available to direct further sequence extension and therefore the polymerase cannot add further nucleotides. This allows convenient detection of single nucleotide incorporation events. Once the identity of the base incorporated into the growing chain has been determined, the 3′ block may be removed to allow addition of the next successive nucleotide. By ordering the products derived using these modified nucleotides, it is possible to deduce the DNA sequence of the DNA template. Multiple reactions can be done in parallel on a single array, for example, if each of the modified nucleotides has a different label attached thereto, known to correspond to the particular base, thereby facilitating discrimination between the bases added during each incorporation step. If desired, a separate reaction may be carried out for each of the modified nucleotides.

Modified nucleotides used in an amplification or sequencing reaction may carry a label to facilitate their detection. A fluorescent label, for example, may be used for detection of modified nucleotides. Each nucleotide type may thus carry a different fluorescent label, for example, as described in WO 2007/135368. The detectable label need not, however, be a fluorescent label. Any label can be used which allows the detection of an incorporated nucleotide. Similarly, fluorescent labels or other labels can be used to detect any of a variety of analytes on an array fabricated using a bead-based transfer method set forth herein.

One method for detecting fluorescently labeled nucleotides comprises using laser light of a wavelength specific for the labeled nucleotides, or the use of other suitable sources of illumination. The fluorescence from the label on the nucleotide may be detected by a CCD camera or other suitable detection means. Suitable instrumentation for recording images of clustered arrays is described in WO 07/123,744. Detectors that are capable of obtaining an image of an array surface such as those configured to scan an array surface. Such detectors can be configured to take a static image of an array surface, scan a point across an array surface or scan a line across an array surface. Exemplary scanning devices that can be used are described, for example, in U.S. Pat. No. 7,329,860. A detector can be configured to obtain an image of an array at high resolution, for example, in the low micron to submicron range. In particular embodiments, an image can be obtained at a Rayleigh resolution between 0.2 and 10 micrometers.

The invention is not intended to be limited to use of the sequencing method outlined above, as a variety of sequencing methodologies which utilize successive incorporation of nucleotides into a nucleic acid chain or removal of nucleotides from a nucleic acid chain can be used. Suitable alternative techniques include, for example, Pyrosequencing, FISSEQ (fluorescent in situ sequencing), MPSS and sequencing by ligation-based methods, for example as described is U.S. Pat. No. 6,306,597. Sequencing by hybridization methods can also be used.

A nucleic acid may be analyzed to obtain a first and then a second sequencing read from opposite ends of the nucleic acid. Methodology for sequencing both ends of nucleic acids at array features (also referred to as “clusters”) are described in WO 07/010,252 and WO 08/041,002. These methods utilize a step of copying a first nucleic acid fragment (or amplicon thereof) by hybridising the 3′ end of this template strand to an immobilized primer followed by extending the resulting bridged structure to generate a second template strand. This copying step can be carried out after the template has been sequenced from a first end. Then the first strand can be cleaved from the surface and the remaining second template strand can be sequenced from the other end. In order to practice this version of the invention, two or more immobilized primers are utilized, at least one of which is configured to be cleavable in order to release the first template strand.

Sequencing can be carried out using other sequencing techniques as well including but not limited to Maxam-Gilbert method, chain-termination methods, high-throughput sequencing, Ladder-based sequencing methods, multiplex sequencing, and sequencing by hybridization. See e.g., U.S. Pat. Nos. 5,674,473; 6,296,810; 7,179,602; 7,272,507; Maxam and Gilbert (1977) Proc. Natl. Acad. Sci. USA 74: 560; Church & Kieffer-Higgins (1988) Science 240: 185-188. Suitable sequencing methods also include a nano method of sequencing as described in Lagerqvist, et al. (2006) Nano Letters 6(4): 779-782. See also Mardis (September 2008) Annual Review of Genomics and Human Genetics 9: 387-402.

Sequencing methods are preferably carried out with the target polynucleotide arrayed on a solid support as exemplified above in regard to sequencing by synthesis methods. Multiple target polynucleotides can be immobilized on the solid support through linker molecules, or can be attached to particles, e.g., microspheres, which can also be attached to a solid support material.

Sequencing methods can be carried out on both single polynucleotide molecule and multi-polynucleotide molecule arrays, e.g., arrays of distinct individual polynucleotide molecules and arrays of distinct regions comprising multiple copies of one individual polynucleotide molecule. Single molecule arrays allow each individual polynucleotide to be resolved separately. The use of single molecule arrays is preferred. Sequencing single molecule arrays non-destructively allows a spatially addressable array to be formed. Brenner, et al. (2000) Nature Biotechnology 18: 630-634; Drmanac, et al. (1992) International Journal of Genome Research 1(1):59-79. An additional technique utilizes sequencing by hybridization. For example, sequencing by hybridization has been described. Drmanac, et al. (1989) Genomics 4:114; Koster, et al. (1996) Nature Biotechnology 14: 1123; U.S. Pat. Nos. 5,525,464; 5,202,231; and 5,695,940. See also Ronaghi, et al., (1998) Science 281: 363-365; Syvanen (1999) Human Mutation 13: 1-10.

In a preferred embodiment, the expression level may be determined by determining the quantity of mRNA.

Methods for determining the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it is advantageous to use nucleic acids in combination with appropriate means, such as a detectable label, for detecting hybridization. A wide variety of appropriate indicators are known in the art including, fluorescent, radioactive, enzymatic, or other ligands (e.g., avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tₘ, e.g., 50% formamide, 5× or 6×SCC. SCC is a 0.15 M NaCl, 0.015 M Na-citrate). The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a micro sphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labeled and contacted with the microarray in hybridization conditions, leading to the formation of complexes between target nucleic acids that are complementary to probe sequences attached to the microarray surface. The labeled hybridized complexes are then detected and can be quantified or semi-quantified. Labeling may be achieved by various methods, e.g. by using radioactive, or fluorescent labeling. Many variants of the microarray hybridization technology are available to the man skilled in the art. See, e.g., the review by Hoheisel in Nature Reviews, Genetics (2006) 7: 200-210.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations) but some experimental errors and deviations should be allowed for. Unless otherwise indicated, parts are parts by weight, molecular weight is average molecular weight, temperature is in degrees centigrade; and pressure is at or near atmospheric.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Identification of Markers Differently Expressed in Oocytes

The processing of oocytes was conducted in a dedicated DNA-free clean-room environment. A total of 27 oocytes, including seven single oocytes (three chromosomally normal and four chromosomally abnormal) and four pooled samples each consisting of five pooled oocytes of unknown chromosomal status were analyzed.

Oocytes were collected in sterile, RNase-free conditions and processed rapidly in order to minimize changes in marker expression. The zona pellucida was removed to ensure the exclusion of all cumulus cells from the sample and the polar body was separated from the oocyte. The oocyte was transferred to a microcentrifuge tube and then immediately frozen, while the polar body was thoroughly washed to remove any DNA contaminants before transfer to a separate microcentrifuge tube.

The polar body DNA was released by lysing the cell. Polar bodies were washed in four 10 μl droplets of phosphate-buffered saline-0.1% polyvinyl alcohol, transferred to a microfuge tube containing 2 μL of proteinase k (125 μg/mL) and 1 μL of sodium dodecyl sulfate (17 μM), and overlaid with oil. Incubation at 37° C. for 1 hour, followed by 15 minutes at 95° C., was done to release the DNA. See Wells, et al. (2002) Fertility and Sterility 78: 543.

The polar body DNA was then amplified using a whole genome amplification method called degenerate oligonucleotide primed PCR (DOP-PCR). Polar-body DNA was amplified using a modification of previously reported methods. Wells, et al. (1999) Nuc Acids Res 27: 1214-1218. Amplification took place in a 50 μL, reaction volume containing the following: 0.2 mM dNTPs; 2.0 μM degenerate oligonucleotide primer, CCGACTCGAGNNNNNNATGTGG; 1× SuperTaq Plus buffer, and 2.5 U of SuperTaq Plus polymerase (Ambion, Austin, Tex.). Thermal cycling conditions were as follows: 94° C. for 4.5 minutes; 8 cycles of 95° C. for 30 seconds, 30° C. for 1 minute, a 1° C./s ramp to 72° C., and 72° C. for 3 minutes; 35 cycles of 95° C. for 30 seconds, 56° C. for 1 minute, and 72° C. for 1.5 minutes; and finally, 72° C. for 8 minutes. After amplification was complete, a 5-μL aliquot of amplified DNA was transferred to a new PCR tube and retained for single-gene testing.

The amplified DNA was used for the purposes of comparative genomic hybridization (CGH), a method that reveals the copy number of every chromosome in the sample. The chromosomes within the polar body are a mirror image of those in the oocyte (e.g., if the polar body has one copy of chromosome 21 too few, the oocyte will have one copy of chromosome 21 too many). Thus, analysis of the polar body indicates whether or not the oocyte is abnormal. Amplified DNA samples (whole-genome amplification products) were precipitated and fluorescently labeled by nick translation. Polar-body DNA was labeled with Spectrum Green-dUTP (Vysis, Downers Grove, Ill.), whereas 46, XX (normal female) DNA was labeled with Spectrum Red-dUTP (Vysis). Both labeled DNAs were precipitated with 30 μg of Cot1 DNA. Precipitated DNA was resuspended in a hybridization mixture composed of 50% formamide; 2× saline sodium citrate [SSC; 20×SSC is 150 mM NaCl and 15 mM sodium citrate, pH 7]; and 10% dextran sulfate). Labeled DNA samples dissolved in hybridization mixture were denatured at 75° C. for 10 minutes, then allowed to cool at room temperature for 2 minutes, before being applied to denatured normal chromosome spreads as described below.

Metaphase spreads from a normal male (46, XY; Vysis) were dehydrated through an alcohol series (70%, 85%, and 100% ethanol for 3 minutes each) and air dried. The slides were then denatured in 70% formamide, 2×SSC at 75° C. for 5 minutes. After this incubation, the slides were put through an alcohol series at −20° C. and then dried. The labeled DNA probe was added to the slides, and a coverslip was placed over the hybridization area and sealed with rubber cement. Slides were then incubated in a humidified chamber at 37° C. for 25-30 hours. After hybridization, the slides were washed sequentially in 2×SSC (73° C.), 4×SSC (37° C.), 4×SSC+0.1% Triton-X (37° C.), 4×SSC (37° C.), and 2×SSC (room temperature); each wash lasted 5 minutes. The slides were then dipped in distilled water, passed through another alcohol series, dried, and finally mounted in anti-fade medium (DAPI II, Vysis) containing diamidophenylindole to counterstain the chromosomes and nuclei.

Fluorescent microscopic analysis allowed the amount of hybridized polar body (green) DNA to be compared with the amount of normal female (red) DNA along the length of each chromosome. Computer software (Applied Imaging, Santa Clara, Calif.) converted these data into a simple red-green ratio for each chromosome; deviations from a 1:1 ratio were indicative of loss or gain of chromosomal material. On the basis of this analysis, oocytes where identified as chromosomally normal or chromosomally abnormal.

RNA was extracted from those oocytes identified as chromosomally normal and from those identified as chromosomally abnormal. This was accomplished using an Absolutely RNA Nanoprep kit (Stratagene) according to the manufacturer's instructions. The RNA from normal and abnormal cells was amplified using a two round in vitro transcription procedure. For this purpose the extracted RNA was subjected to reverse transcription (RT), primed using an oligo(dT) primer containing a phage T7 RNA Polymerase promoter sequence at its 5′-end. First strand cDNA synthesis was catalyzed by SuperScript® III Reverse Transcriptase (Invitrogen) and performed at an elevated temperature to reduce RNA secondary structure. The RNA of the cDNA:RNA hybrid produced during RT was digested into small RNA fragments using an RNase H enzyme. The RNA fragments primed second strand cDNA synthesis. The resulting double-stranded cDNA contained a T7 transcription promoter in an orientation that will generate anti-sense RNA (aRNA; also called nRNA) during a subsequent in vitro transcription reaction. High yields of aRNA were produced in a rapid in vitro transcription reaction that utilized a T7 RNA polymerase and the double-stranded cDNA produced in the previous step. The aRNA produced was then purified by spin column chromatography. This initial round of reverse transcription and in vitro RNA synthesis was undertaken using a TargetAmp kit (Epicentre Biotechnologies). A second round of reverse transcription, second strand cDNA synthesis and in vitro transcription was accomplished using a NanoAmp RT-IVT labeling kit (Applied Biosystems), following the manufacturer's recommended protocol. During the second round of amplification labeled nucleotides were incorporated into the RNA, permitting subsequent chemiluminescent detection after hybridization to a microarray. The amplification process produced up to 21 μg of RNA per oocyte. The fragments produced were up to 10 kb in size (mean fragment size—500 bp).

An Applied Biosystems Human Genome Survey Microrray was used to analyze RNA expression. This microarray has 32,878 probes for the interrogation of 29,098 genes. The chemiluminescent detection system of this microarray provides a great dynamic range that allows for the detection of rare transcripts and reliable identification of subtle variations in expression level. This microarray, and information about this particular microarray, is available from Applied Biosystems. Expression analysis was performed using Panther software (Applied Biosystems, CA) and Spotfire.

Human oocytes were found to express over 12,400 markers. Of these, 6,226 markers appeared to be expressed consistently and have been detected in all samples assessed. A comparison of chromosomally normal oocytes with chromosomally abnormal oocytes revealed a total of 308 markers displaying a significant difference in expression level (46 markers p<0.01; 262 markers p<0.05). Of the markers displaying statistically significant differences in expression between chromosomally normal oocytes and chromosomally abnormal oocytes, those showing the greatest fold differences in expression are listed in TABLE 2.

TABLE 2
depicts a list of markers differentially expressed between chromosomally normal and chromosomally abnormal oocytes.

| Exemplified | | | | | | |
|---|---|---|---|---|---|---|
| by SEQ | Entrez | Ensembl | Gene | | abnormal/ | |
| ID NOs | GeneID | GeneID | Symbol | Gene Name | normal | p-value |
| 1, 93 | 2533 | ENSG00000082074 | FYB | FYN binding protein (FYB-120/130) | 0.1962 | 0.00046 |
| 2, 94 | 440073 | ENSG00000120645 | IQSEC3 | IQ motif and Sec7 domain 3 | 1.6546 | 0.00063 |
| 3, 95 | 29097 | ENSG00000143771 | CNIH4 | cornichon homolog 4 | 0.1966 | 0.00088 |
| 4, 96 | 340419 | ENSG00000147655 | RSPO2 | R-spondin 2 homolog | 1.7716 | 0.00099 |
| 5, 97 | 9851 | ENSG00000198920 | KIAA0753 | KIAA0753 | 0.0784 | 0.00114 |
| 6, 98 | 222484 | ENSG00000139517 | LNX2 | Ligand of numb-protein X 2 | 0.3123 | 0.00161 |
| 7 | 284475 | — | LOC284475 | hypothetical protein | 0.4473 | 0.00243 |
| 8, 99 | 126789 | ENSG00000169972 | PUSL1 | pseudouridylate synthase-like 1 | 1.7901 | 0.00274 |
| 9, 100 | 113179 | ENSG00000213638 | ADAT3 | adenosine deaminase, tRNA-specific 3 | 0.2204 | 0.00279 |
| 10, 101 | 3621 | ENSG00000153487 | ING1 | inhibitor of growth family, member 1 | 12.2589 | 0.00388 |
| 11, 102 | 55611 | ENSG00000167770 | OTUB1 | OTU domain, ubiquitin aldehyde binding 1 | 0.4151 | 0.00391 |
| 12, 103 | 5081 | ENSG00000009709 | PAX7 | paired box gene 7 | 2.7515 | 0.00447 |
| 13, 104 | 5919 | ENSG00000106538 | RARRES2 | retinoic acid receptor responder (tazarotene | 2.5706 | 0.00476 |
| 14, 105 | 25963 | ENSG00000103978 | TMEM87A | transmembrane protein 87A | 2.8555 | 0.00494 |
| 15, 106 | 501 | ENSG00000164904; | ALDH7A1 | aldehyde dehydrogenase 7 family, member Al | 0.4209 | 0.00522 |
| 16, 107 | 8394 | ENSG00000143398 | PIP5K1A | phosphatidylinositol-4-phosphate 5-kinase, | 0.4273 | 0.03590 |
| 17, 108 | 9254 | ENSG00000007402 | CACNA2D2 | calcium channel, voltage-dependent, alpha | 4.4399 | 0.00533 |
| 18, 109 | 54033 | ENSG00000185272 | RBM11 | RNA binding motif protein 11 | 0.3571 | 0.00534 |
| 19, 110 | 84643 | ENSG00000141200 | KIF2B | kinesin family member 2B | 3.6393 | 0.00570 |
| 20, 111 | 84074 | ENSG00000129646 | QRICH2 | glutamine rich 2 | 2.6744 | 0.00579 |
| 21, 112 | 4759 | ENSG00000115488 | NEU2 | sialidase 2 (cytosolic sialidase) NEU2 | 2.6492 | 0.00637 |
| 22, 113 | 25923 | ENSG00000184743 | ALTA3 | DKFZP564j0863 protein | 0.0703 | 0.00678 |
| 23, 114 | 83657 | ENSG00000168589 | DYNLRB2 | dynein, cytoplasmic, light polypeptide 2B | 2.6787 | 0.00791 |
| 24, 115 | 144501 | ENSG00000167767 | KRT80 | keratin 80 | 0.7072 | 0.00815 |
| 25, 116 | 5162 | ENSG00000168291 | PDHB | pyruvate dehydrogenase (lipoamide) beta | 0.2264 | 0.00910 |
| 26, 117 | 10533 | ENSG00000197548 | ATG7 | ATG7 autophagy related 7 homolog | 0.5964 | 0.00935 |
| 27, 118 | 5558 | ENSG00000146143 | PRIM2 | primase, polypeptide 2A, 58 kDa | 2.4069 | 0.00970 |
| 28, 119 | 10097 | ENSG00000138071 | ACTR2 | ARP2 actin-related protein 2 homolog | 3.4179 | 0.01011 |
| 29, 120 | 91433 | ENSG00000166965 | RCCD1 | RCC1 domain containing 1 | | |
| 30, 121 | 339287 | ENSG00000188895 | MSL-1 | male-specific lethal-1 homolog | 0.2941 | 0.01145 |
| 31, 122 | 51514 | ENSG00000143476 | DTL | denticleless homolog | 5.3455 | 0.01253 |
| 32, 123 | 91768 | ENSG00000134508 | CABLES1 | Cdk5 and Abl enzyme substrate 1 | 1.8754 | 0.01308 |
| 33, 124 | 345611 | — | IRGM | immunity-related GTPase family, M | 3.1934 | 0.01334 |
| 34, 125 | 23788 | ENSG00000109919 | MTCH2 | mitochondrial carrier homolog 2 | 0.3084 | 0.01354 |
| 35, 126 | 5125 | ENSG00000099139 | PCSK5 | proprotein convertase subtilisin/kexin type 5 | 5.0740 | 0.01542 |
| 36, 127 | 64064 | ENSG00000198754 | OXCT2 | 3-oxoacid CoA transferase 2 | 0.5209 | 0.01546 |
| 37, 128 | 1843 | ENSG00000120129 | DUSP1 | dual specificity phosphatase 1 | 0.0849 | 0.01594 |
| 38, 129 | 10970 | ENSG00000136026 | CKAP4 | cytoskeleton-associated protein 4 | 0.2647 | 0.01598 |
| 39, 130 | 128240 | ENSG00000163382 | AP0A1BP | apolipoprotein A-I binding protein | 3.6208 | 0.01609 |
| 40, 131 | 84109 | ENSG00000186867 | GPR103 | G protein-coupled receptor 103 | 0.3496 | 0.01685 |
| 41, 132 | 90338 | ENSG00000170949 | ZNF160 | zinc finger protein 160 | 0.2457 | 0.01733 |
| 42, 133 | 9708 | ENSG00000214574 | PCDHGA8 | protocadherin gamma subfamily A, 8 | 1.8571 | 0.01774 |
| 43, 134 | 5201 | ENSG00000113068 | PFDN1 | prefoldin 1 | 4.5350 | 0.01812 |
| 44, 135 | 23761 | ENSG0000,0100141 | PISD | phosphatidylserine decarboxylase | 0.5412 | 0.01912 |
| 45, 136 | 7277 | ENSG00000127824 | TUB4A | tubulin, alpha 1 (testis specific) | 1.5449 | 0.01937 |
| 46, 137 | 23705 | ENSG00000182985 | CADMI | immunoglobulin superfamily, member 4 | 2.3015 | 0.01955 |
| 47, 138 | 9373 | ENSG00000137055 | PLAA | phospholipase A2-activating protein | 0.4540 | 0.02028 |
| 48, 139 | 27242 | ENSG00000146072 | TNFRSF21 | tumor necrosis factor receptor superfamily, | 0.4338 | 0.02048 |
| 49 | 439991 | — | LOC439991 | similar to D-PCa-2 protein | 1.3575 | 0.02243 |
| 50, 140 | 219988 | ENSG00000166889 | PATL1 | topoisomerase II-associated protein | 0.3673 | 0.02299 |
| 51, 141 | 5797 | ENSG00000173482 | PTPRM | protein tyrosine phosphatase, receptor type, M | 0.2815 | 0.02410 |
| 52, 142 | 10497 | ENSG00000198722 | UNC13B | unc-13 homolog B | 0.3288 | 0.02429 |
| 53, 143 | 10461 | ENSG00000153208 | MERTK | c-mer proto-oncogene tyrosine kinase | 0.3436 | 0.02470 |
| 54, 144 | 1997 | ENSG00000120690 | ELF1 | E74-like factor 1 (ets domain transcription | 0.2396 | 0.02496 |
| 55, 145 | 56604 | ENSG00000127589 | TUBB4Q | tubulin, beta polypeptide 4, member Q | 1.8208 | 0.02637 |
| 56, 146 | 5223 | ENSG00000171314 | PGAM1 | phosphoglycerate mutase 1 (brain) | 0.1760 | 0.03014 |
| 57, 147 | 5395 | ENSG00000122512 | PMS2 | postmeiotic segregation increased 2 | | |
| 58, 148 | 56203 | ENSG00000163380 | LMOD3 | leiomodin 3 (fetal) | 0.3314 | 0.03082 |
| 59, 149 | 2101 | ENSG00000173153 | ESRRA | estrogen-related receptor alpha | 4.7284 | 0.03175 |
| 60, 150 | 373861 | ENSG00000188662 | HILS1 | histone linker H1 domain, spennatid-specific 1 | 0.3991 | 0.03194 |
| 61, 151 | 4212 | ENSG00000134138 | MEIS2 | Meis homeobox 2 | 0.3137 | 0.03345 |
| 62, 152 | 5636 | ENSG00000141127 | PRPSAP2 | phosphoribosyl pyrophosphate synthetase- | 0.1847 | 0.03423 |
| 63, 153 | 114827 | ENSG00000142621 | FHAD1 | forkhead-associated (FHA) phosphopeptide | 4.4247 | 0.03436 |
| 64, 154 | 92181 | ENSG00000168246 | UBTD2 | ubiquitin domain containing 2 | 0.2467 | 0.03498 |
| 65, 155 | 7067 | ENSG00000126351 | THRA | thyroid hormone receptor, alpha | 0.1797 | 0.03562 |
| 66, 156 | 139741 | ENSG00000123165 | ACTRT1 | actin-related protein T1 | 0.3739 | 0.03563 |
| 67, 157 | 55544 | ENSG00000132819 | RBM38 | RNA binding motif protein 38 | 4.1900 | 0.03581 |
| 68, 158 | 6388 | ENSG00000132581 | SDF2 | stromal cell-derived factor 2 | 0.2185 | 0.03615 |
| 69, 159 | 168975 | ENSG00000176571 | CNBD1 | cyclic nucleotide binding domain containing 1 | 0.1701 | 0.03643 |
| 70, 160 | 56257 | ENSG00000146834 | MEPCE | bin3, bicoid-interacting 3, homolog | 3.3279 | 0.03645 |
| 71, 161 | 55831 | ENSG00000125037 | TMEM111 | transmembrane protein 111 | 5.2241 | 0.03702 |
| 72, 162 | 5782 | ENSG00000127947 | PTPN12 | protein tyrosine phosphatase, non-receptor | 0.2185 | 0.03824 |
| 73, 163 | 7336 | ENSG00000169139 | UBE2V2 | ubiquitin-conjugating enzyme E2 variant 2 | 0.2215 | 0.04004 |
| 74, 164 | 22849 | ENSG00000107864 | CPEB3 | cytoplasmic polyadenylation element binding | 2.5130 | 0.04203 |
| 75, 165 | 8366 | ENSG00000124529 | HIST1H4B | histone 1, H4b | 2.4576 | 0.04207 |
| 76, 166 | 254531 | ENSG00000176454 | AGPAT7 | 1-acylglycerol-3-phosphate 0-acyltransferase | 3.3807 | 0.04318 |
| 77, 167 | 81704 | ENSG00000107099 | DOCK8 | dedicator of cytokinesis 8 | 0.2787 | 0.04387 |
| 78, 168 | 143689 | ENSG00000134627 | PIWIL4 | piwi-like 4 | 0.2890 | 0.04439 |
| 79, 169 | 10336 | ENSG00000185619 | PCGF3 | polycomb group ring finger 3 | 4.8836 | 0.04479 |
| 80, 170 | 3158 | ENSG00000134240 | HMGCS2 | 3-hydroxy-3-methylglutaryl-Coenzyme A | 0.4403 | 0.04533 |
| 81, 171 | 259266 | ENSG00000066279 | ASPM | asp (abnormal spindle)-like, microcephaly | 0.2942 | 0.04578 |
| 82, 172 | 9948 | ENSG00000071127 | WDR1 | WD repeat domain 1 | 2.2581 | 0.04632 |
| 83, 173 | 83985 | ENSG00000169682 | SPNSI | Spinster | 0.4638 | 0.04716 |
| 84, 174 | 51741 | ENSG00000186153 | WWOX | WW domain containing oxidoreductase | 4.9246 | 0.04778 |
| 85, 175 | 1576 | ENSG00000160868 | CYP3A4 | cytochrome P450, family 3, subfamily A, | 3.1504 | 0.04855 |
| 86, 176 | 9238 | ENSG00000136270 | TBRG4 | transforming growth factor beta regulator 4 | 0.3539 | 0.04901 |
| 87, 177 | 84666 | ENSG00000163515 | RETNLB | resistin like beta | 0.1210 | 0.00918 |
| 88, 178 | 10487 | ENSG00000131236 | CAP1 | CAP, adenylate cyclase-associated protein 1 | 2.1734 | 0.01121 |
| 89, 179 | 23175 | ENSG00000134324 | LPIN1 | lipin 1 | 0.4955 | 0.03730 |
| 90, 180 | 4850 | ENSG00000080802 | CNOT4 | CCR4-NOT transcription complex, subunit 4 | 9.1233 | 0.04524 |
| 91, 181 | 120329 | — | CASP12 | caspase 12 | 0.2563 | 0.03776 |
| 92, 182 | 6611 | ENSG00000102172 | SMS | spermine synthase | 0.1262 | 0.02887 |
| | — | — | — | Probe 131701 hybridizing Celera | | |
| | | | | hCG2040689 | | |
| | — | | — | Probe 228780 hybridizing Celera | | |
| | | | | hCG1987894 | | |
| | — | — | — | Probe 705174 hybridizing Celera | | |
| | | | | hCG2043502 | | |
| | — | — | — | Probe 233419 hybridizing Celera | | |
| | | | | hCG1644979 | | |

GeneID is a unique identifier assigned to a record in Entrez Gene. Entrez Gene provides these tracked, unique identifiers for genes and reports information associated with those identifiers for unrestricted public use at the National Center for Biotechnology Information Website. Ensembl GeneID is a unique stable gene identifier of the Ensembl database, publicly available at the Ensembl Genome Browser. See Hubbard, et al. (2007) Nucleic Acids Res. 35 (Database Issue): D610-7.

Several of the differentially expressed markers are known or suspected to be involved in the maintenance of accurate chromosome segregation, including checkpoint genes and microtubule motor proteins and may have fundamental roles in the genesis of aneuploidy in human oocytes. For example, abnormal expressed of TUBA1 was observed in aneuploid oocytes. Mutations in this gene destabilize spindle microtubules, potentially leading to chromosome malsegregation. Abnormal expression of dynein and kinesin genes (e.g. DNCL2B and KIF2B genes) was also observed and may be significant given the role of the protein products of such genes in facilitating chromosomal movement.

Although some of the pathways implicated have been suggested to be involved in the genesis of meiotic chromosome error, surprisingly, none of the specific differentially expressed markers identified have been the subject of investigation for meiotic chromosome error. For example, it has been speculated that mitochondrial dysfunction could lead to problems with chromosome segregation, possibly due to ATP depletion. However, the mitochondrial (or mitochondrion-related) genes highlighted in this study (e.g., MTCH2, HMGCS2) have not previously been suggested to have a role in aneuploidy.

The markers having traditionally attracted the most attention as potential candidates for regulating meiotic chromosome malsegregation, appear by this analysis to be of lesser importance. For example, well-characterized genes functioning in the metaphase-anaphase (spindle) checkpoint (e.g., BUB1 and MAD2) were not found to show altered expression in aneuploid oocytes, while lesser studied genes with potential roles in cell cycle control displayed significant differences in gene expression, such as ASP (abnormal spindle-like, microcephaly associated.) and UBE2V2.

Several markers involved in nucleoside metabolism and cholesterol biosynthesis were differentially expressed between chromosomally normal and chromosomally abnormal oocytes, including PRPSAP2 and CYP3A4. This suggests that these processes might be directly or indirectly involved with chromosome malsegregation during female meiosis.

Several markers are located on the cell surface, or are excreted proteins, or are involved in biosynthetic pathways that affect levels of excreted metabolites were differentially expressed between chromosomally normal and chromosomally abnormal oocytes. For example, genes for cell surface receptor proteins, TNFRSF21, PTPRM, ESRRA, GPR103 and THRA.

### Example 2

### Identification of Markers Differently Expressed in Cumulus Cells

The processing of cumulus cells was conducted in a dedicated DNA-free clean-room environment. A total of six cumulus cells (three chromosomally normal and three chromosomally abnormal) were analyzed.

Cumulus cells were collected in sterile, RNase-free conditions. The cumulus cells were separated from the oocyte mechanically and processed rapidly in order to minimize changes in marker expression. The zona pellucida was removed from the corresponding oocyte and the polar body was separated. The oocyte was transferred to a microcentrifuge tube and then immediately frozen, while the polar body was thoroughly washed to remove any DNA contaminants before transfer to a separate microcentrifuge tube.

The polar body DNA was released by lysing the cell. Polar bodies were washed in four 10 μL droplets of phosphate-buffered saline-0.1% polyvinyl alcohol, transferred to a microfuge tube containing 2 μL of proteinase k (125 μg/mL) and 1 μL of sodium dodecyl sulfate (17 μM), and overlaid with oil. Incubation at 37° C. for 1 hour, followed by 15 minutes at 95° C., was done to release the DNA. See Wells, et al. (2002) Fertility and Sterility 78: 543.

The polar body DNA was then amplified using a whole genome amplification method called degenerate oligonucleotide primed PCR (DOP-PCR). Polar-body DNA was amplified using a modification of previously reported methods. Wells, et al. (1999) Nuc Acids Res 27: 1214-1218. Amplification took place in a 50-μL reaction volume containing the following: 0.2 mM dNTPs; 2.0 μM degenerate oligonucleotide primer, CCGACTCGAGNNNNNNATGTGG; 1× SuperTaq Plus buffer, and 2.5 U of SuperTaq Plus polymerase (Ambion, Austin, Tex.). Thermal cycling conditions were as follows: 94° C. for 4.5 minutes; 8 cycles of 95° C. for 30 seconds, 30° C. for 1 minute, a 1° C./s ramp to 72° C., and 72° C. for 3 minutes; 35 cycles of 95° C. for 30 seconds, 56° C. for 1 minute, and 72° C. for 1.5 minutes; and finally, 72° C. for 8 minutes. After amplification was complete, a 5-μL aliquot of amplified DNA was transferred to a new PCR tube and retained for single-gene testing.

The amplified DNA was used for the purposes of comparative genomic hybridization (CGH), a method that reveals the copy number of every chromosome in the sample. The chromosomes within the polar body are a mirror image of those in the oocyte (e.g. if the polar body has one copy of chromosome 21 too few, the oocyte will have one copy of chromosome 21 too many). Thus, analysis of the polar body indicates whether or not the oocyte is abnormal. Amplified DNA samples (whole-genome amplification products) were precipitated and fluorescently labeled by nick translation. Polar-body DNA was labeled with Spectrum Green-dUTP (Vysis, Downers Grove, Ill.), whereas 46, XX (normal female) DNA was labeled with Spectrum Red-dUTP (Vysis). Both labeled DNAs were precipitated with 30 μg of Cot1 DNA. Precipitated DNA was resuspended in a hybridization mixture composed of 50% formamide; 2× saline sodium citrate [SSC; 20×SSC is 150 mM NaCl and 15 mM sodium citrate, pH 7]; and 10% dextran sulfate). Labeled DNA samples dissolved in hybridization mixture were denatured at 75° C. for 10 minutes, then allowed to cool at room temperature for 2 minutes, before being applied to denatured normal chromosome spreads as described below.

Metaphase spreads from a normal male (46, XY; Vysis) were dehydrated through an alcohol series (70%, 85%, and 100% ethanol for 3 minutes each) and air dried. The slides were then denatured in 70% formamide, 2×SSC at 75° C. for 5 minutes. After this incubation, the slides were put through an alcohol series at −20° C. and then dried. The labeled DNA probe was added to the slides, and a coverslip was placed over the hybridization area and sealed with rubber cement. Slides were then incubated in a humidified chamber at 37° C. for 25-30 hours. After hybridization, the slides were washed sequentially in 2×SSC (73° C.), 4×SSC (37° C.), 4×SSC+0.1% Triton-X (37° C.), 4×SSC (37° C.), and 2×SSC (room temperature); each wash lasted 5 minutes. The slides were then dipped in distilled water, passed through another alcohol series, dried, and finally mounted in anti-fade medium (DAPI II, Vysis) containing diamidophenylindole to counterstain the chromosomes and nuclei.

Fluorescent microscopic analysis allowed the amount of hybridized polar body (green) DNA to be compared with the amount of normal female (red) DNA along the length of each chromosome. Computer software (Applied Imaging, Santa Clara, Calif.) converted these data into a simple red-green ratio for each chromosome; deviations from a 1:1 ratio were indicative of loss or gain of chromosomal material. On the basis of this analysis, oocytes and their associated cumulus cells were identified as chromosomally normal or chromosomally abnormal.

RNA was extracted from those cumulus cells associated with chromosomally normal oocytes and from those associated with oocytes that were chromosomally abnormal. This was accomplished using an Absolutely RNA Nanoprep kit (Stratagene) according to the manufacturer's instructions. The RNA was amplified using a two round in vitro transcription procedure. For this purpose the extracted RNA was subjected to reverse transcription (RT), primed using an oligo(dT) primer containing a phage T7 RNA Polymerase promoter sequence at its 5′-end. First strand cDNA synthesis was catalyzed by SuperScript® III Reverse Transcriptase (Invitrogen) and performed at an elevated temperature to reduce RNA secondary structure. The RNA of the cDNA:RNA hybrid produced during RT was digested into small RNA fragments using an RNase H enzyme. The RNA fragments primed second strand cDNA synthesis. The resulting double-stranded cDNA contained a T7 transcription promoter in an orientation that will generate anti-sense RNA (aRNA; also called tRNA) during a subsequent in vitro transcription reaction. High yields of aRNA were produced in a rapid in vitro transcription reaction that utilized a T7 RNA polymerase and the double-stranded cDNA produced in the previous step. The aRNA produced was then purified by spin column chromatography. This initial round of reverse transcription and in vitro RNA synthesis was undertaken using a TargetAmp kit (Epicentre Biotechnologies).

A second round of reverse transcription, second strand cDNA synthesis and in vitro transcription was accomplished using a NanoAmp RT-IVT labeling kit (Applied Biosystems), following the manufacturer's recommended protocol. During the second round of amplification labeled nucleotides were incorporated into the RNA, permitting subsequent chemiluminescent detection after hybridization to a microarray. The amplification process produced up to 154 μg of RNA per cumulus cell. The fragments produced were up to 10 kb in size (mean fragment size −500 bp).

An Applied Biosystems Human Genome Survey Microrray was used to analyze RNA expression. This microarray has 32,878 probes for the interrogation of 29,098 genes. The chemiluminescent detection system of this microarray provides a great dynamic range that allows for the detection of rare transcripts and reliable identification of subtle variations in expression level. This microarray, and information about this particular microarray, is available from Applied Biosystems. Expression analysis was performed using Panther software (Applied Biosystems, CA) and Spotfire.

Human cumulus cells were found to express over 8,000 markers. Of these, 3,350 markers appeared to be expressed consistently and have been detected in all samples assessed. A comparison of chromosomally normal cumulus cells with chromosomally abnormal cumulus cells revealed 752 markers displaying a significant difference in expression level (125 markers p<0.01; 627 markers p<0.05).

Of the markers displaying statistically significant differences in expression between chromosomally normal cumulus cells and chromosomally abnormal cumulus cells, those with the greatest fold differences in expression are listed in TABLE 3.

TABLE 3
depicts a list of markers differentially expressed between cumulus cells associated with chromosomally normal
oocytes and cumulus cells associated with chromosomally abnormal oocytes.

| Exemplified | | | | | | |
|---|---|---|---|---|---|---|
| by SEQ ID | Entrez | Ensembl | Gene | | abnormal/ | |
| NOS: | GeneID | GeneID | Symbol | Gene Name | normal | p-value |
| 183, 283 | 4070 | ENSG00000184292 | TACSTD2 | tumor-associated calcium signal transducer 2 | 0.0575 | 0.00390 |
| 184, 284 | 5930 | ENSG00000122257 | RBBP6 | retinoblastoma binding protein 6 | 0.1509 | 0.00104 |
| 185, 285 | 148789 | ENSG00000162885 | B3GALNT2 | beta-1,3-N-acetylgalactosaminyltransferase 2 | 0.1520 | 0.00256 |
| 186, 286 | 79680 | ENSG00000215012 | FLJ21125 | chromosome 22 open reading frame 29 | 0.1526 | 0.00269 |
| 187, 287 | 6360 | ENSG00000161573 | CCL16 | chemokine (C—C motif) ligand 16 | 0.1580 | 0.00031 |
| 188, 288 | 1660 | ENSG00000135829 | DHX9 | DEAH (Asp-Glu-Ala-His) box polypeptide 9 | 0.1818 | 0.00714 |
| 189, 289 | 79075 | ENSG00000136982 | DCC1 | defective in sister chromatid cohesion homolog | 0.2015 | 0.00150 |
| 190, 290 | 55186 | ENSG00000114120 | SLC25A36 | solute carrier family 25, member 36 | 0.2200 | 0.00970 |
| 191, 291 | 285761 | ENSG00000164465 | DCBLD1 | discoidin, CUB and LCCL domain containing | 0.2311 | 0.00195 |
| 192, 292 | 55752 | ENSG00000138758 | SEPT11 | septin 11 | 0.2345 | 0.00892 |
| 193, 293 | 55593 | ENSG00000068308 | OTUD5 | OTU domain containing 5 | 0.2430 | 0.00730 |
| 194, 294 | 284047 | ENSG00000154874 | CCDC144B | coiled-coil domain containing 144B | 0.1529 | 0.01005 |
| 195, 295 | 3592 | ENSG00000168811 | IL12A | interleukin 12A | 0.0575 | 0.00390 |
| 196, 296 | 56675 | ENSG00000175352 | NRIP3 | nuclear receptor interacting protein 3 | 0.2843 | 0.00012 |
| 197, 297 | 136319 | ENSG00000105887 | MTPN | myotrophin | 0.3123 | 0.00508 |
| 198, 298 | 1464 | ENSG00000173546 | CSPG4 | chondroitin sulfate proteoglycan 4 | 0.3199 | 0.00274 |
| 199, 299 | 80149 | ENSG00000163874 | ZC3H12A | zinc finger CCCH-type containing 12A | 0.3347 | 0.00019 |
| 200, 300 | 56143 | ENSG00000204965 | PCDHA5 | protocadherin alpha 5 | 0.3401 | 0.00673 |
| 201, 301 | 8703 | ENSG00000158850 | B4GALT3 | UDP-Gal:betaGlcNAc beta 1,4- | 0.3414 | 0.00595 |
| 202, 302 | 116225 | ENSG00000165724 | ZMYND19 | zinc forger, MYND-type containing 19 | 0.3479 | 0.00889 |
| 203, 303 | 143425 | ENSG00000170743 | SYT9 | synaptotagmin IX | 0.3569 | 0.00040 |
| 204, 304 | 7027 | ENSG00000198176 | TFDP1 | transcription factor Dp-1 | 0.3621 | 0.00752 |
| 205, 305 | 55132 | ENSG00000138709 | LARP2 | La ribonucleoprotein domain family, member | 0.3740 | 0.00070 |
| 206, 306 | 8837 | ENSG00000003402 | CFLAR | CASP8 and FADD-like apoptosis regulator | 0.3798 | 0.00502 |
| 207, 307 | 51280 | ENSG00000135052 | GOLM1 | golgi membrane protein 1 | 0.3875 | 0.00093 |
| 208, 308 | 11202 | ENSG00000129455 | KLK8 | kallikrein-related peptidase 8 | 0.3899 | 0.00844 |
| 209, 309 | 3921 | ENSG00000168028 | RPSA15 | LOC388524 ribosomal protein SA | 0.3936 | 0.00603 |
| 210, 310 | 55182 | ENSG00000187147 | Clorf164 | chromosome 1 open reading frame 164 | 0.3983 | 0.00400 |
| 211, 311 | 23543 | ENSG00000100320 | RBM9 | RNA binding motif protein 9 | 0.4030 | 0.00599 |
| 212, 312 | 127253 | ENSG00000162623 | TYW3 | tRNA-yW synthesizing protein 3 homolog | 0.4182 | 0.00868 |
| 213, 313 | 594 | ENSG00000083123 | BCKDHB | branched chain keto acid dehydrogenase E1, | 0.4224 | 0.00851 |
| 214, 314 | 64663 | ENSG00000198573 | SPANXC | SPANX family, member C | 0.4278 | 0.00115 |
| 215 | — | ENSG00000187832 | RPSA | ribosomal protein SA | 0.4380 | 0.00662 |
| 216, 315 | 115752 | ENSG00000166938 | DIS3L | DIS3 mitotic control homolog | 0.4479 | 0.00445 |
| 217, 316 | 9894 | ENSG00000100726 | TELO2 | TEL2, telomere maintenance 2, homolog | 0.4513 | 0.00911 |
| 218, 317 | 1019 | ENSG00000135446 | CDK4 | cyclin-dependent kinase 4 | 0.4608 | 0.00131 |
| 219, 318 | 7169 | ENSG00000198467 | TPM2 | tropomyosin 2 (beta) | 0.4612 | 0.00251 |
| 220, 319 | 127406 | ENSG00000188819 | LOC127406 | similar to laminin receptor 1 | 0.4623 | 0.00963 |
| 221, 320 | 91272 | ENSG00000145919 | FAM44B | family with sequence similarity 44, member B | 0.4627 | 0.00728 |
| 222, 321 | 171392 | ENSG00000197372 | ZNF675 | zinc finger protein 675 | 0.4648 | 0.00732 |
| 223, 322 | 308 | ENSG00000164111 | ANXAS | annexin A5 | 0.4667 | 0.00530 |
| 224, 323 | 3340 | ENSG00000070614 | NDST1 | N-deacetylase/N-sulfotransferase (heparan | 0.4723 | 0.00603 |
| 225, 324 | 85459 | ENSG00000166004 | KIAA1731 | KIAA1731 | 0.4781 | 0.00723 |
| 226, 325 | 10482 | ENSG00000162231 | NXF1 | nuclear RNA export factor 1 | 0.4793 | 0.00267 |
| 227, 326 | 65987 | ENSG00000151364 | KCTD14 | potassium channel tetramerisation domain | 0.4817 | 0.00567 |
| 228, 327 | 11186 | ENSG00000068028 | RASSF1 | Ras association (Ra1GDS/AF-6) domain family | 0.4852 | 0.00142 |
| 229, 328 | 197021 | ENSG00000188501 | LCTL | lactase-like | 0.4984 | 0.00832 |
| 230, 329 | 3206 | ENSG00000153807 | HOXA1O | homeobox A10 | 3.6454 | 0.00537 |
| 231, 330 | 200261 | — | LOC200261 | LOC200261 | 0.0535 | 0.01506 |
| 232, 331 | 23126 | ENSG00000143442 | POGZ | pogo transposable element with ZNF domain | 0.0590 | 0.01359 |
| 233, 332 | 60677 | ENSG00000140488 | BRUNOL6 | bruno-like 6, RNA binding protein | 0.1172 | 0.03122 |
| 234, 333 | 6935 | ENSG00000148516 | ZEB1 | zinc finger E-box binding homeobox 1 | 0.1395 | 0.01642 |
| 235, 334 | 55075 | ENSG00000137831 | UACA | uveal autoantigen with coiled-coil domains and | 0.1587 | 0.01652 |
| 236, 335 | 9473 | ENSG00000130775 | C1orf38 | chromosome 1 open reading frame 38 | 0.1603 | 0.04512 |
| 237, 336 | 54576 | ENSG00000167165 | UGT1A9 | UDP glucuronosyltransferase 1 family, | 0.1631 | 0.02645 |
| 238, 337 | — | ENSG00000187667 | WHDC1L1 | WAS protein homology region 2 domain | 0.1695 | 0.04314 |
| 239, 338 | 440253 | — | WHDC1L2 | WAS protein homology region 2 domain | 0.1695 | 0.04314 |
| 240, 339 | 63895 | ENSG00000154864 | FAM38B | FAM38B | 0.1712 | 0.02929 |
| 241, 340 | 55088 | ENSG00000165813 | C10orf118 | ClOorf118 | 0.1808 | 0.03736 |
| 242, 341 | 983 | ENSG00000170312 | CDC2 | cyclin-dependent kinase 1 | 0.1944 | 0.01489 |
| 243, 342 | 6558 | ENSG00000064651 | SLC12A2 | solute carrier family 12 | 0.1949 | 0.02259 |
| 244, 343 | 54456 | ENSG00000073146 | MOVIOL1 | Moloney leukemia virus 10-like 1 | 0.2002 | 0.04357 |
| 245, 344 | 5332 | ENSG00000101333 | PLCB4 | phospholipase C, beta 4 | 0.2124 | 0.02353 |
| 246, 345 | 23519 | ENSG00000139223 | ANP32D | acidic (leucine-rich) nuclear phosphoprotein 32 | 0.2140 | 0.02994 |
| 247, 346 | 653121 | ENSG00000160062 | ZBTB8 | zinc finger and BTB domain containing 8 | 0.2162 | 0.03949 |
| 248, 347 | 245802 | ENSG00000166926 | MS4A6E | membrane-spanning 4-domains, subfamily A, | 0.2182 | 0.03753 |
| 249, 348 | 341392 | ENSG00000215009 | LOC341392 | acyl-CoA synthetase medium-chain family | 0.2317 | 0.02346 |
| 250, 349 | 140688 | ENSG00000197183 | C20orf112 | chromosome 20 open reading frame 112 | 0.2338 | 0.01827 |
| 251, 350 | 57730 | ENSG00000187998 | LOC375251 | KIAA1641-like | 0.2346 | 0.02143 |
| 252, 351 | 84727 | ENSG00000111671 | SPSB2 | splA/ryanodine receptor domain and SOCS | 0.2368 | 0.03253 |
| 253, 352 | 55212 | ENSG00000138686 | BBS7 | Bardet-Biedl syndrome 7 | 0.2411 | 0.02927 |
| 254, 353 | 2048 | ENSG00000133216 | EPHB2 | EPH receptor B2 | 0.2478 | 0.01074 |
| 255, 354 | 54979 | ENSG00000133328 | HRASLS2 | HRAS-like suppressor 2 | 0.2577 | 0.01007 |
| 256, 355 | 81543 | ENSG00000160233 | LRRC3 | leucine rich repeat containing 3 | 0.2606 | 0.01893 |
| 257, 356 | 9662 | ENSG00000174799 | CEP135 | centrosomal protein 135 kDa | 0.2617 | 0.04571 |
| 258, 357 | 124222 | ENSG00000162073 | PAQR4 | progestin and adipoQ receptor family member | 0.2715 | 0.03814 |
| 259, 358 | 54790 | ENSG00000168769 | KIAA1546 | KIAA1546 | 0.2721 | 0.04948 |
| 260, 359 | 7357 | ENSG00000148154 | UGCG | UDP-glucose ceramide glucosyltransferase | 0.2721 | 0.01173 |
| 261, 360 | 1553 | ENSG00000197838 | CYP2A13 | cytochrome P450, family 2, subfamily A, | 0.2725 | 0.04184 |
| 262, 361 | 10893 | ENSG00000125966 | MMP24 | matrix metallopeptidase 24 (membrane- | 0.2739 | 0.04106 |
| 263, 362 | 26267 | ENSG00000147912 | FBXO10 | F-box protein 10 | 0.2741 | 0.04768 |
| 264, 363 | 10450 | ENSG00000084072 | PPIE | peptidylprolyl isomerase E (cyclophilin E) | 0.2774 | 0.03494 |
| 265, 364 | 222166 | ENSG00000180354 | C7orf41 | chromosome 7 open reading frame 41 | 0.2815 | 0.03485 |
| 266, 365 | 29989 | ENSG00000171102 | OBP2B | odorant binding protein 2B | 0.2857 | 0.03155 |
| 267, 366 | 2318 | ENSG00000128591 | FLNC | filamin C, gamma (actin binding protein 280) | 0.2862 | 0.03826 |
| 268, 367 | 84313 | ENSG00000131475 | VPS25 | vacuolar protein sorting 25 homolog | 0.2878 | 0.03393 |
| 269, 368 | 59353 | ENSG00000126950 | TMEM35 | transmembrane protein 35 | 0.2885 | 0.02098 |
| 270, 369 | 9992 | ENSG00000159197 | KCNE2 | potassium voltage-gated channel, Isk-related | 0.2892 | 0.04245 |
| 271, 370 | 375748 | ENSG00000182150 | LOC375748 | RAD26L hypothetical protein | 0.2907 | 0.02333 |
| 272, 371 | 9486 | ENSG00000115526 | CHST10 | carbohydrate sulfotransferase 10 | 0.2948 | 0.03819 |
| 273, 372 | 51491 | ENSG00000048162 | U384 | HSPC111 | 0.2960 | 0.02762 |
| 274, 373 | 55219 | ENSG00000204178 | TMEM57 | transmembrane protein 57 | 0.2977 | 0.02216 |
| 275, 374 | 222236 | ENSG00000161048 | NAPE-PLD | N-acyl-phosphatidylethanolamine-hydrolyzing | 0.3009 | 0.01231 |
| 276, 375 | 55055 | ENSG00000174442 | ZWILCH | Zwilch, kinetochore associated, homolog | 0.3016 | 0.01698 |
| 277, 376 | 220136 | ENSG00000172361 | CCDC11 | coiled-coil domain containing 11 | 0.3065 | 0.03578 |
| 278, 377 | 4884 | ENSG00000171246 | NPTX1 | neuronal pentraxin I | 0.3088 | 0.03202 |
| 279, 378 | 23075 | ENSG00000133789 | SWAP70 | SWAP-70 protein | 0.3097 | 0.03146 |
| 280, 379 | 10396 | ENSG00000124406 | ATP8A1 | ATPase, aminophospholipid transporter | 0.3107 | 0.03182 |
| 281, 380 | 2624 | ENSG00000179348 | GATA2 | GATA bindingprotein 2 | 0.1712 | 0.02929 |
| 282 | — | ENSG00000183427 | — | ENSG00000183427 | 0.4291 | 0.00588 |

GeneID is a unique identifier assigned to a record in Entrez Gene. Entrez Gene provides these tracked, unique identifiers for genes and reports information associated with those identifiers for unrestricted public use at the National Center for Biotechnology Information Website. Ensembl GeneID is a unique stable gene identifier of the Ensembl database, publicly available at the Ensembl Genome Browser. See Hubbard, et at. (2007) Nucleic Acids Res. 35 (Database Issue): D610-7.

### Example 3

### Methodology for Assessment of Oocyte Competence

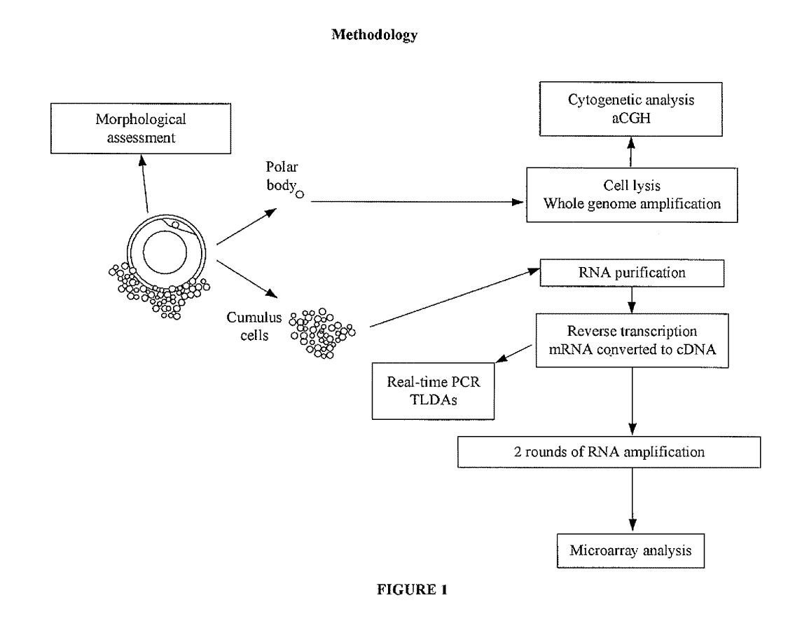

To evaluate the competence of an oocyte for implantation or fertilization, a morphological assessment may be made of the oocyte, cytogenetic analysis of the polar body, and/or microarray analysis of cumulus cells associated with the oocyte. See FIG. 1.

The oocyte may be removed from the cumulus cells and polar body associated with it for morphological assessment and karyotyping.

The polar body may be removed from the oocyte and cumulus cells in which it is associated, the cells lysed and whole genome amplification followed by cytogenetic analysis via array comparative genomic hybridization (aCGH) to detect genomic copy number variations at a higher resolution level than chromosome-based genomic hybridization (CGH).

The cumulus cells may be removed from the oocyte and polar body. The RNA may be purified from the cumulus cells and reverse transcription of the mRNA into cDNA. An aliqout of the cDNA may undergo real-time PCR and the Taqman Low Density Array (TLDA) for gene expression analysis. Another aliqout of the cDNA may undergo at least about two rounds of RNA amplification and then microarray analysis. For example, nucleic acid from a test sample and normal reference sample are labeled differentially, using different fluorophores, and hybridized to several thousand probes. The probes are derived from most of the known genes and non-coding regions of the genome, printed on a glass slide. The ratio of the fluorescence intensity of the test to that of the reference nucleic acid is then calculated, to measure the copy number changes for a particular location in the genome. Using this method, copy number changes at a level of 5-10 kilobases of DNA sequences can be detected. The high-resolution CGH (HR-CGH) arrays may detect structural variations (SV) at resolution of 200 bp. This method allows one to identify new recurrent chromosome changes such as microdeletions and duplications in found in birth defects due to chromosome aberrations. See Urban, et al. (2006) Proc. Natl. Acad. Sci. 103: 4534-39.

Cytogenetic Results:

The aCGH results from 26 first PBs (from 9 patients) with an average maternal age: 38.3 years (age range: 30-46 years) comprising 36 years or less: 3 women (av. age: 32.7 years) and 37 years or more: 6 women (av. age: 41.2 years) yielded 13 PBs/oocytes normal after aCGH and 13 PBs/oocytes abnormal after aCGH. The abnormal chromosome samples showed whole chromosome non-disjunction and unbalanced chromatid predivision as well as chromosomes of all sizes affected by aneuploidy

Cumulus Cell Gene Expression:

The cumulus cells (CCs) from 3 normal and 3 aneuploid oocytes assessed including 3 patients, age range: 32-41 years, average age: 38 years. These samples showed lower mRNA quantities in CCs from aneuploid oocytes. In total, 29,098 genes were examined with 17,388 genes consistently expressed in all cumulus cell investigated and 729 genes exhibiting significant differences in expression between aneuploid and normal groups (606 P<0.05, 123 P<0.01). Of these 729 genes, 272 genes were over-expressed and 457 genes were under-expressed. The microarray results were validated by via real-time PCR using an independent set of samples and TaqMan low density arrays (TLDAs) that allows for a simultaneous analysis of large numbers of genes. Of these genes examined, 94 genes were selected from array data and 2 housekeeping genes for further analysis.

The cumulus cells from 10 normal and 10 aneuploid oocytes were examined including 6 patients, age range: 30-46 years, average age: 38. 7 years. 60 out of the 94 genes were concordant with the microarray data. Of note, 14 genes (e.g., B3GALNT2, C22orf29, CCL16, DCBLD1, DCC1, DHX9, OTUD5, RBBP6, SEPT11, SLC25A36, SPSB2, TACSTD2, Unassigned, and TP53I3) exhibited highly significant differential expression (P<0.01) with a large mRNA copy number fluctuations (FC>4) and were down-regulated in cumulus cells of aneuploid oocytes. These genes include receptors, transferases, signaling molecules, nucleic acid, binding, cytoskeletal proteins, and carrier proteins.

Conclusions:

The inventors used arrays and real-time PCR to conduct a comprehensive analysis of cumulus cell transcriptome allowing for an insight into follicular microenvironment of aneuploid oocytes. Aneuploid oocytes are associated with transcriptionally quiescent and less proliferative cumulative cells. Further, the abnormal expression of genes regulating metabolism, cell-cell communication, hypoxia, and apoptosis was found. Thus there is a relationship between follicular microenvironment and oocyte aneuploidy. In particular, fourteen genes are useful targets for non-invasive test development (e.g., B3GALNT2, C22orf29, CCL16, DCBLD1, DCC1, DHX9, OTUD5, RBBP6, SEPT11, SLC25A36, SPSB2, TACSTD2, Unassigned, and TP53I3) For example, the SPSB2 gene (e.g., UniProtKB Q99619) expression in cumulus cells correlated with oocyte chromosome status and potential to lead to live birth.

### Example 4

### Oocyte Chromosome Status Determined by Cumulus Cell Analysis

Oocyte chromosome status was determined by first polar body (PB) biopsy, followed by analysis using a well-validated microarray comparative genomic hybridization (aCGH) method. This approach allowed identification of aneuploidy affecting any chromosome in the corresponding oocyte with high accuracy. CCs were removed mechanically prior to PB biopsy. Following RNA extraction, and RNA amplification, cumulus cell gene expression was quantified. Initially, cumulus cell gene expression was analyzed via microarray, providing an assessment of over 29,000 separate gene transcripts. Results were later confirmed for 94 candidate genes using reverse transcription followed by real-time polymerase chain reaction, a process considered to be the gold standard for quantification of mRNA transcript copy numbers.

### Results

### Cytogenetic Analysis of First PBs

Microarray comparative genomic hybridization (aCGH) is a DNA-based method, which provides data on the relative copy number of every chromosomal region in a single experiment. Because aCGH analysis of single PBs involves placing the cells intact in tubes, artifacts that affect methods such as fluorescent in situ hybridization, which require the spreading of PBs on microscope slides, are eliminated. Hence the aCGH approach for PB analysis is both reliable and also comprehensive. High accuracy was confirmed by the recent ESHRE-sponsored proof of principle study that examined this technology. Geraedts (2010) Hum Reprod 25(Suppl 1): i17-i18.

A total of 26 first polar bodies biopsied from oocytes donated from 9 women of an average age of 38.3 years (range: 32-46 years), who were undergoing IVF due to male factor infertility and tubal problems (i.e., no ovarian involvement) were analyzed. aCGH was successful for all 26 first PBs. Cumulus cells were also collected from these oocytes.

A total of 13 first polar bodies and therefore their corresponding oocytes were characterized as being abnormal, with the remainder being classified as normal. The abnormal oocytes were derived from 8 women, whose average age was 39.4 years (age range: 32-46 years). Analysis of the obtained data confirmed the presence of two main mechanisms leading to maternal aneuploidy; whole chromosome non-disjunction and unbalanced chromatid predivision. Chromosomes of all sizes were found to participate in aneuploidy events. The results from the aCGH analysis of the 13 aneuploid first PBs are shown in Table 4.

TABLE 4
Molecular karyotypes of the 13 aneuploid polar bodies and gene expression
analysis methodology for the corresponding cumulus cells.

| | | | | | Cumulus Cell |
|---|---|---|---|---|---|
| | | | | | gene |
| | Maternal | Polar Body | Polar Body | Oocyte | expression |
| Patient no. | Age | Number | molecular karyotype | characterization | analysis |
| 1 | 32 | 3 | 24, X, +16 | Abnormal | Microarray |
| 2 | 36 | 3 | 24, X, +X | Abnormal | Real-time PCR |
| | | 4 | 23, X, +4cht, +5cht, +7, −15, | Abnormal | Real-time PCR |
| | | | −20cht | | |
| | | 6 | 22, X, −18cht, −21 | Abnormal | Real-time PCR |
| 3 | 39 | 3 | 23, X, −13cht | Abnormal | Real-time PCR |
| | | 4 | 22, X, −3 | Abnormal | Real-time PCR |
| 4 | 39 | 1 | 22, X, −15 | Abnormal | Real-time PCR |
| | | 3 | 24, X, +20 | Abnormal | Real-time PCR |
| 5 | 40 | 7 | 22, X, −22 | Abnormal | Microarray |
| 6 | 41 | 1 | 24, X, +17 | Abnormal | Microarray |
| 7 | 42 | 1 | 24, X, +15, −18, −19cht, | Abnormal | Real-time PCR |
| | | | +20 | | |
| | | 5 | 23, X, −16cht, +21cht | Abnormal | Real-time PCR |
| 8 | 46 | 3 | 23, X, +8cht, +19cht | Abnormal | Real-time PCR |

Gene Expression Analysis of CCs from Normal and Aneuploid Oocytes

Clumps of cumulus cells (30-50 cells) isolated from three normal and three abnormal MII oocytes were subjected to expression microarray analysis. The microarray employed was the Human Genome Survey Microarray (Applied Biosystems). A linear form of amplification, via an in vitro transcription reaction using a highly specific T7 RNA polymerase promoter, was used to produce sufficient mRNA amounts from the cumulus cell clumps for subsequent microarray analysis. For this purpose two different commercially available in vitro transcription kits (TargetAmp 2-round aRNA amplification kit 2.0, Epicentre; NanoAmp RT-IVT labeling kit, Applied Biosystems) were combined. After extensive optimization and validation of the methodology, the amplification protocol was applied to samples of cumulus cells, successfully generating 13-154 μg of RNA (average ˜91 μg), and fragment sizes ranging from less than 50 nucleotides to over 4,000 nucleotides in length, as revealed by analysis with an Agilent Bioanalyzer. Interestingly, the mRNA amount in the cumulus cell (CC) clumps isolated from the aneuploid MII oocytes tended to be lower (13-83 μg, average 39 μg) than that observed for cumulus cell clumps isolated from the chromosomally normal oocytes (132-154 μg, average 143 μg). This finding suggested that CCs enclosing aneuploid oocytes could be less transcriptionally active, compared to those surrounding chromosomally normal oocytes.

Microarray analysis allowed for the definition of a typical cumulus cell transcriptome, cataloguing all genes expressed in human cumulus cells. Specifically, utilizing a conservative definition for the positive detection of expression for individual genes (signal-to-noise ratio>3), 17,388 of the 29,098 sequences examined were consistently expressed in every cumulus cell sample assessed. The gene expression patterns seen were very similar for all of the cumulus cell clumps examined. However, 729 genes displayed statistically (P<0.05) differential expression levels in CCs derived from the aneuploid versus those derived from the normal oocytes. Of these, a total of 457 genes were down-regulated in the CCs from the aneuploid group, and 272 were up-regulated. Highly significant alterations (P<0.01) in expression patterns were observed for 123 of the above genes, with 91 being down-regulated and 32 being up-regulated.

Bioinformatic analysis, including classification of expressed genes according to molecular functions and biological processes was accomplished using the PANTHER tool, a database which contains a reference list of all known human genes (Applied Biosystems). Comparisons of differentially expressed gene groups within the PANTHER database demonstrated a highly disproportionate down-regulation of genes encoding for ribosomal, and other nucleic acid binding proteins, transcription factors and other signaling molecules, receptors, transferases and cytoskeletal proteins, in the cumulus cells of the aneuploid oocytes. Conversely, genes encoding proteins involved in ion channels, cell junctions and oxidoreductases were observed to be mostly up-regulated in the cumulus cells of the aneuploid oocytes, versus the ones from the normal oocytes. Together, these genes were participating in the regulation of over 30 biological processes, including signal transduction, translational regulation, protein biosynthesis, metabolism and modification, cell adhesion and communication, membrane traffic, and homeostasis. Additionally, a total of 245 previously uncharacterized genes were shown to be differentially expressed, 113 of which were down-regulated and 132 were up-regulated.

In order to verify the results obtained from the microarray analysis, a real-time PCR method known as a TaqMan Low Density Array (TLDA, Applied Biosystems) was used. TLDAs are able to simultaneously analyze a larger numbers of genes, compared with conventional real-time PCR approaches, and provide highly accurate quantification of mRNA transcripts. Hence, it was possible to further examine the expression of a total of 96 of the 729 differentially expressed genes, including 2 house-keeping genes (GADPH and HPRT1). Cumulus cells were taken from 10 normal and 10 aneuploid oocytes, categorized after aCGH analysis of the first polar body. The data obtained was normalized using the endogenous house-keeping genes as a control (results were essentially the same regardless of the housekeeping gene used). Of the 94 selected genes, 60 showed expression profiles which were confirmatory of the microarray data. Of these, 14 genes showed highly significant expression differences (P<0.01) and large mRNA transcript copy number fluctuations (>4) between cumulus cells of normal and aneuploid oocytes, during both the microarray and the TLDA experiments. All these 14 genes were down-regulated in the CCs surrounding aneuploid oocytes, and their names and functions are summarized in Table 5.

TABLE 5
Details of the functions of the 14 genes with significantly different expression in
cumulus cells of normal and aneuploid oocytes.

| | | | | | Exemplary |
|---|---|---|---|---|---|
| | | | | | Nucleic Acid/ |
| | Gene | | | | Amino Acid |
| Gene name | Symbol | Gene group | Molecular function | Biological process | (SEQ ID NO) |
| Beta-1,3-N-acetylgalactosaminyltransferase 2 | B3GALNT2 | Transferase | Glycosyltransferase | Protein metabolism | 185/285 |
| Chromosome 22 open reading frame 29 | C22orf29 | Receptor | G-protein coupled | G-protein mediated | 381/382 |
| | | | receptor | signaling; Cell | |
| | | | | proliferation and | |
| | | | | differentiation | |
| Chemokine (C-C motif) ligand 16 | CCL16 | Signalling | Chemokine | Ligand-mediated | 187/287 |
| | | molecule | | signaling | |
| Discoidin, CUB and LCCL domain containing 1 | DCBLD1 | Unclassified | Unclassified | Unclassified | 191/291 |
| Defective in sister chromatid cohesion | DCC1 | Replication origin | Chromatin binding | DNA replication; Cell | 189/289 |
| homolog 1 (S. cerevisiae) | | binding protein | | proliferation and | |
| | | | | differentiation | |
| DEAH (Asp-Glu-Ala-His) box polypeptide 9 | DHX9 | Helicase | RNA helicase | mRNA splicing | 188/288 |
| OTU domain containing 5 | OTUD5 | Ribosomal | Nucleic acid Binding | Sex determination; | 193/293 |
| | | protein | | chromosome segregation | |
| Retinoblastoma binding protein 6 | RBBP6 | Nuclease | Nuclease | mRNA polyadenylation; | 184/284 |
| | | | | Other metabolism | |
| Septin 11 | Septin 11 | Cytoskeletal | Small GTPase/ | Cytokinesis/Apoptosis | 192/292 |
| | | protein | Cytoskeletal protein | | |
| Solute carrier family 25, member 36 | SLC25A36 | Transfer/carrier | Mitochondrial carrier | Small molecule transport | 190/290 |
| | | protein | protein | | |
| SPRY domain-containing SOCS box protein 2 | SPSB2 | Signalling | | Intracellular signaling | 252/351 |
| | | molecule | | cascade/Ubiquitination/ | |
| | | | | Cell surface receptor | |
| | | | | mediated signal | |
| | | | | transduction | |
| Tumor-associated calcium signal transducer 2 | TACSTD2 | Receptor | Receptor | Cell surface receptor | 383/384 |
| | | | | mediated signal | |
| | | | | transduction; Cell | |
| | | | | proliferation and | |
| | | | | differentiation | |
| Unassigned | Unassigned | Helicase | RNA helicase | mRNA splicing | |
| Tumor protein p53 | TP5313 | | Oxidoreductase | Apoptosis; | 385/386 |
| inducible protein 3 | | | activity | Carbohydrate metabolic | 387/388 |
| | | | | process | 389/390 |

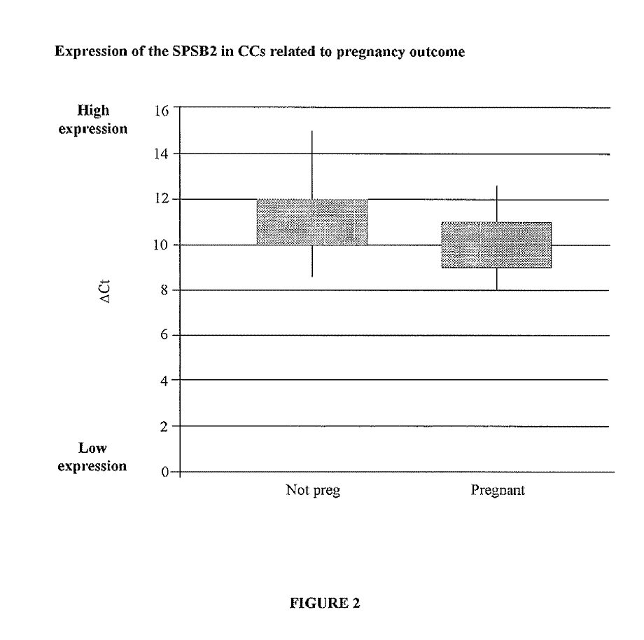

14 genes were assessed in relation to clinical outcome (i.e., the establishment of a pregnancy leading to a healthy live birth) in a new set of samples. This part of the study took place in a blinded manner, and was achieved by investigating the expression profiles of the 14 genes in a set of 38 CCs corresponding to embryos that were transferred without any chromosome screening. Of these 38 embryos, 18 implanted and produced live births and 20 failed to produce a clinical pregnancy. Among the 14 genes examined, SPSB2 and TP53I3 showed the clearest association with live birth, tending to be up-regulated in the cumulus cells of oocytes which led to successful pregnancies (FIG. 2). These differences in expression between the two sample groups approached statistical significance (P=0.055).

### Example 5

### SPSB2 and TP53I3 Gene Expression Analysis

Materials and Methods

Patients and Samples

This research was conducted using Institutional Review Board approved protocols and with patient signed informed consent. A total of 28 karyotypically normal women participated in this research project. All these women were undergoing ART treatment with or without PGS in three IVF clinics in the USA. These women were aged between 23 and 46 years (average age 32.8 years).

The patient and sample details of this research are in Table 6 below.

TABLE 6
Patient and sample details. The method used for gene expression analysis is also included.

| Patient | Maternal age | | Oocytes | | Type of poly-A mRNA |
|---|---|---|---|---|---|
| no. | (years) | Indication for ART | processed | No. of CCs examined | analysis |
| 1 | 23 | Unknown | 2 | 2 | TLDAs |
| 2 | 26 | Oocyte donor | 1 | 1 | TLDAs |
| 3 | 27 | Oocyte donor | 1 | 1 | TLDAs |
| 4 | 27 | Endometriosis, Right | 1 | 1 | TLDAs |
| | | Oophorectomy | | | |
| 5 | 28 | Unknown | 2 | 2 | TLDAs |
| 6 | 29 | Endometriosis | 3 | 3 | TLDAs |
| 7 | 30 | Oocyte donor | 5 | 5 inner CC layers | Real-time PCR |
| 8 | 30 | Unknown | 1 | 1 | TLDAs |
| 9 | 30 | LPD, Oligospermia | 2 | 2 | TLDAs |
| 10 | 30 | Oligospermia | 1 | 1 | TLDAs |
| 11 | 30 | Unknown | 2 | 2 | TLDAs |
| 12 | 31 | Oligospermia, Dysmenorrhea | 2 | 2 | TLDAs |
| 13 | 31 | LPD, Dysmenorrhea | 1 | 1 | TLDAs |
| 14 | 33 | Oligospermia | 2 | 2 | TLDAs |
| 15 | 33 | Tubal Factor | 1 | 1 | TLDAs |
| 16 | 34 | Dysmenorrhea | 2 | 2 | TLDAs |
| 17 | 34 | Oligospermia | 2 | 2 | TLDAs |
| 18 | 35 | Unknown | 2 | 2 | TLDAs |
| 19 | 35 | Oligospermia, Dysmenorrhea | 3 | 3 | TLDAs |
| 20 | 35 | Secondary Infertility, Right | 2 | 2 | TLDAs |
| | | Tubal Occlusion | | | |
| 21 | 35 | Amenorrhea | 2 | 2 | TLDAs |
| 22 | 35 | Unknown | 2 | 2 | TLDAs |
| 23 | 35 | Amenorrhea, Hypogonadism | 1 | 1 | TLDAs |
| 24 | 36 | Male factor | 4 | 4 outer and 4 inner CC layers | TLDAs & Real-time PCR |
| 25 | 39 | PGS* due to AMA** | 5 | 4 outer and 5 inner CC layers | TLDAs & Real-time PCR |
| 26 | 41 | Male factor | 3 | 3 inner CC layers | Real-time PCR |
| 27 | 42 | PGS due to AMA | 6 | 3 outer and 6 inner CC layers | TLDAs & Real-time PCR |
| 28 | 46 | PGS due to AMA | 3 | 2 outer and 3 inner CC layers | TLDAs & Real-time PCR |

*PGS—Preimplantation genetic screening
**AMA—Advanced maternal age

Table 6 shows a summary of the details of this patient group, along with the number of CC samples examined and the type of technique used for mRNA analysis. For patients 7, 24, 25, 26, 27 and 28 (Table 7), the ovarian stimulation protocol consisted of GnRH agonist started in midluteal phase of the previous cycle and recombinant FSH (rFSH). Subsequent oocyte collection took place as described previously (Fragouli et al., 2010). For the remaining patients, the employed stimulation protocols were designed according to what the treating clinicians thought would be the most suited, including a combination of either GnRH agonist or antagonist with recombinant FSH, with or without hMG. Serum estradiol levels and transvaginal ultrasound were used to monitor ovarian response. After administration of hCG for the final follicular maturation, the oocytes were collected 36 hours later.

### Oocyte, PBs and Cumulus Cells

A total of 26 oocytes (patients 7, and 24-28 in Table 6) had their first PBs biopsied and cytogenetically analyzed with the use of microarray comparative genomic hybridization (aCGH). In addition to the PBs, two clumps of CCs were also removed from 13 of the oocytes. One CC sample consisted of part of the outer layer of the cumulus-oocyte-complex (COC) and the other was taken from the inner layer, in close contact with the oocyte. For the remaining 13 oocytes of this group only the inner layer of CCs was sampled. An additional 38 oocytes (patients 1-6 and 8-23 in Table 7) had clumps of CCs removed mostly from the inner CC layer, but no cytogenetic analysis of first PBs took place for this group of samples. Gene expression in a total of 51 CC clumps was assessed using TaqMan low density arrays (TLDAs) to investigate up to 96 genes per sample, whereas the inner CC layers (26 sets of cell clumps) were used for a more focused real-time PCR experiment assessing just three genes (details described in subsequent sections).

### Sample Processing

Micromanipulation of PBs and CCs took place in sterile conditions with solutions and equipment treated to remove/inactivate RNA degrading enzymes. Shortly before IVF procedures took place, part of the outer CC layer was removed mechanically with the use of stripper pipettes (51 samples). In some cases (see above) part of the inner layer of CCs was also removed after a brief exposure to hyaluronidase (26 samples). Each CC clump was briefly washed in phosphate buffered saline (Invitrogen, USA) containing 0.1% w/v polyvinyl alcohol (Sigma, USA) and 0.4 U/ml RNasin Plus RNase inhibitor (Promega, USA). The CC clumps were subsequently transferred to microcentrifuge tubes and then immediately frozen and stored at −80° C. until RNA extraction took place. The collection of CCs was performed rapidly so as to minimize mRNA degradation or any induced changes in gene expression. First PBs were biopsied from mature metaphase II oocytes after removal of CCs and prepared for aCGH analysis as described previously (Fragouli et al. 2011a; 2011b).

aCGH Analysis of PBs

The chromosome complement of biopsied first PBs was examined using aCGH. The procedure took place as described in Fragouli et al., (2011a). Briefly, whole genome amplification was carried out using the SurePlex amplification kit (Rubicon, USA/BlueGnome Ltd, UK) according to the manufacturer's instructions. Amplified DNA from the first PB and previously amplified normal male (46,XY) DNA (SureRef; BlueGnome Ltd, UK) were fluorescently labelled with the use of the Fluorescence Labelling System (BlueGnome Ltd, UK), also according to the manufacturer's instructions. ‘Test’ PB DNA was labelled in Cy3 and the ‘reference’ 46,XY DNA was labelled with Cy5. Test and reference DNA co-precipitation, denaturation, array hybridization and post-hybridization washes all took place as suggested by the manufacturer. The hybridization time was 16 h.

### Scanning and Image Analysis and Interpretation

A laser scanner (InnoScan 710, Innopsys, Carbonne, France) was used to excite the fluorophores, and to read and store the resulting hybridization images. The MAPIX software (Innopsys, France) was used to control the scanning of the microarray slides. The resulting images were stored in TIFF format file and were analyzed by the BlueFuse Multi analysis software (BlueGnome Ltd, UK), using criteria and algorithms recommended by the manufacturer of the software (Bluegnome Ltd, UK). First PBs and their corresponding oocytes were classified as normal or aneuploid according to these criteria and algorithms (BlueGnome Ltd, UK).

### Cumulus Cell RNA Extraction

Extraction of RNA for all 51 CC clumps analyzed using TLDAs occurred with the use of the PicoPure RNA Isolation kit (Arcturus, UK). The procedure took place as suggested by the manufacturer with a few modifications. The extracted RNA was eluted to a final volume of 15-30 μl of elution buffer and sample concentration along with the absorption at 260/280 and 260/230 were measured twice, using a Nanodrop spectrophotometer. All samples were stored at −80° C. until TLDAs analysis was carried out. The Quick Extract RNA extraction kit (Epicentre, Cambio, UK) was employed to extract RNA from the 26 samples composed of inner CC layers. These samples subsequently underwent a smaller scale real-time PCR analysis. The Quick Extract RNA extraction protocol followed was a modified version of that described by the manufacturer. Specifically, the amount of the QuickExtract RNA extraction Solution was reduced tenfold, so as to adjust volumes for the subsequent step of cDNA synthesis. It was not possible to measure the concentrations for this set of samples, as almost the entirety of the extracted RNA was used for cDNA synthesis and real-time PCR.

cDNA Synthesis from Total RNA

RNA was converted to cDNA using the High Capacity cDNA Reverse Transcription kit (Applied Biosystems, UK). In samples with measured concentrations, a total of up to 16 ng of extracted RNA was used per sample. The total sample volume was adjusted to 10 μl with the addition of DEPC water. The rest of the reaction included 2 μl of 10×RT Buffer, 0.8 μl of 25×dNTP Mix (100 mM), 2 μl of 10×RT Random primers, 1 μl of MultiScribe Reverse Transcriptase, and 4.2 μl of nuclease-free H₂O per sample. cDNA synthesis took place by incubating the samples at 25° C. for 10 minutes and at 37° C. for 120 minutes.

### CC Gene Expression Analysis Via TLDAs

The expression of 96 genes was evaluated in 51 CC clumps (Tables 6 and 7) using TaqMan Low Density Arrays, a microvolume real-time PCR technology (Applied Biosystems, UK).

TABLE 7
Molecular karyotypes of the 26 biopsied first PBs and gene expression analysis methodology
for the corresponding CC's

| | | | | | | CC gene |
|---|---|---|---|---|---|---|
| Patient | PB | PB molecular | Oocyte | Outer | Inner | expression |
| no. | no. | karyotype | characterization | CCs | CCs | analysis |
| 7 | 7-1 | 23, X | Normal | No | Yes | Real-time PCR |
| | 7-2 | 23, X | Normal | No | Yes | |
| | 7-3 | 23, X | Normal | No | Yes | |
| | 7-4 | 23, X | Normal | No | Yes | |
| | 7-5 | 23, X | Normal | No | Yes | |
| 24 | 24-2 | 23, X | Normal | Yes | Yes | TLDAs & |
| | 24-3 | 23, X | Normal | Yes | Yes | Real-time PCR |
| | 24-4 | 23, X, +4cht, +5cht, +7, −15, −20cht | Abnormal | Yes | Yes | |
| | 24-6 | 22, X, −18cht, −21 | Abnormal | Yes | Yes | |
| 25 | 25-1 | 22, X, −15 | Abnormal | Yes | Yes | TLDAs & |
| | 25-2 | 23, X | Normal | Yes | Yes | Real-time PCR |
| | 25-3 | 24, X, +20 | Abnormal | Yes | Yes | |
| | 25-4 | 23, X | Normal | Yes | Yes | |
| | 25-5 | 22, X, +10cht, +15cht, −18 | Abnormal | No | Yes | |
| 26 | 26-1 | 24, X, +19 | Abnormal | No | Yes | Real-time PCR |
| | 26-2 | 23, X, −10cht | Abnormal | No | Yes | |
| | 26-3 | 23, X | Normal | No | Yes | |
| 27 | 27-1 | 24, X, +15, −18, −19cht, +20 | Abnormal | Yes | Yes | TLDAs & |
| | 27-2 | 23, X | Normal | Yes | Yes | Real-time PCR |
| | 27-3 | 23, X | Normal | No | Yes | |
| | 27-4 | 23, X | Normal | Yes | Yes | |
| | 27-5 | 23, X, −16cht, +21cht | Abnormal | No | Yes | |
| | 27-7 | 23, X | Normal | No | Yes | |
| 28 | 28-1 | 23, X | Normal | Yes | Yes | TLDAs & |
| | 28-3 | 23, X, +8cht, +19cht | Abnormal | Yes | Yes | Real-time PCR |
| | 28-4 | 23, X | Normal | No | Yes | |

TABLE 8
ΔCt values for TP53I3 and SPSB2 in inner CC layers removed from normal and aneuploid
oocytes. Low ΔCt values correspond to gene up-regulation, whereas high ΔCt values
correspond to gene down-regulation

| Patient no. | CC no. | Oocyte characterisation | TP53I3 ΔCt | SPSB2 ΔCt | TP53I3 and SPSB2 status in CCs |
|---|---|---|---|---|---|
| 7 | 7-1-CC | Normal | 1.57 | 5.721 | Down-regulated |
| | 7-2-CC | Normal | −0.486 | 4 | Up-regulated |
| | 7-3-CC | Normal | 0.26 | 5.825 | Down-regulated |
| | 7-4-CC | Normal | No result | No result | No result |
| | 7-5-CC | Normal | No result | No result | No result |
| 24 | 24-2-CC | Normal | −0.153 | 3.353 | Up-regulated |
| | 24-3-CC | Normal | −0.575 | 1.964 | Up-regulated |
| | 24-4-CC | Abnormal | 2.115 | 6.932 | Down-regulated |
| | 24-6-CC | Abnormal | No result | No result | No result |
| 25 | 25-1-CC | Abnormal | 0.526 | 5.326 | Down-regulated |
| | 25-2-CC | Normal | 1.422 | 7.066 | Down-regulated |
| | 25-3-CC | Abnormal | 1.666 | 5.403 | Down-regulated |
| | 25-4-CC | Normal | 2.109 | 6.958 | Down-regulated |
| | 25-5-CC | Abnormal | 1.915 | 6.588 | Down-regulated |
| 26 | 26-1-CC | Abnormal | 1.962 | 4.078 | Down-regulated |
| | 26-2-CC | Abnormal | 2.558 | 5.161 | Down-regulated |
| | 26-3-CC | Normal | 2.153 | 5.09 | Down-regulated |
| 27 | 27-1-CC | Abnormal | No result | No result | No result |
| | 27-2-CC | Normal | −1.074 | 2.891 | Up-regulated |
| | 27-3-CC | Normal | −0.401 | 2.51 | Up-regulated |
| | 27-4-CC | Normal | −0.214 | 2.393 | Up-regulated |
| | 27-5-CC | Abnormal | No result | No result | No result |
| | 27-7-CC | Normal | −1.194 | 1.213 | Up-regulated |
| 28 | 28-1-CC | Normal | 0.805 | 5.885 | Down-regulated |
| | 28-3-CC | Abnormal | 0.544 | 6.544 | Down-regulated |
| | 28-4-CC | Normal | −0.506 | 6.027 | Up-regulated |

Samples with ‘No result’ are likely to be due to loss of RNA during the extraction procedure or failure of reverse transcription, since no cDNA amplification was possible for any gene.

TABLE 9
Details of the 58 CC genes associated with aneuploidy following TLDA analysis.

| | | | | | Expression | | |
|---|---|---|---|---|---|---|---|
| | | | | | in | | |
| | | | | | CCs of | | |
| | | | | | aneuploid | | |
| | | | | | oocytes | | |
| | | | | | compared | Relative | |
| | | | | | to CCs | expression | |
| | Gene | | | Biological | of normal | aneuploid/ | |
| Gene name | Symbol | Gene group | Molecular function | process | oocytes | euploid | P-value |
| Annexin A5 | ANXA5 | Transfer/carrier | Annexin, | Lipid, fatty acid | Under- | 0.47 | P < 0.01 |
| | | protein | Transfer/carrier | and steroid | expressed | | |
| | | | protein | metabolism | | | |
| Beta-1,3-N- | B3GALNT2 | Transferase | Glycosyltransferase | Protein metabolic | Under- | 0.15 | P < 0.01 |
| acetylgalactosaminyl- | | | | process | expressed | | |
| transferase 2 | | | | | | | |
| Bardet-Biedl | BBS7 | Membrane Traffic | Unclassified | Unclassified | Under- | 0.24 | P < 0.05 |
| syndrome 7 | | | | | expressed | | |
| BMSI homolog, | BMS1L | Nucleic acid | Unclassified | Nucleobase, | Under- | 0.39 | P < 0.05 |
| ribosome | | binding | | nucleoside, | expressed | | |
| assembly protein | | | | nucleotide and | | | |
| (yeast) | | | | nucleic acid | | | |
| | | | | metabolic process | | | |
| Breast cancer 1, | BRCA1 | Zinc finger | Zinc finger | mRNA | Under- | 0.36 | P < 0.05 |
| early onset | | protein | transcription factor | transcription | expressed | | |
| | | | | regulation/Tumor | | | |
| | | | | suppressor/Cell | | | |
| | | | | cycle control/ | | | |
| | | | | DNA repair | | | |
| Bruno-like 6, | BRUNOL6 | Nucleic acid | Other RNA-binding | mRNA splicing | Under- | 0.12 | P < 0.05 |
| RNA binding | | binding | protein | | expressed | | |
| protein | | | | | | | |
| (Drosophila) | | | | | | | |
| Chromosome 22 | C22orf29 | Receptor | G-protein coupled | G-protein | Under- | | P < 0.01 |
| open reading | | | receptor | mediated | expressed | | |
| frame 29 | | | | signalling/Cell | | | |
| | | | | proliferation and | | | |
| | | | | differentiation | | | |
| Capping protein | CAPZA3 | Cytoskeletal | Non-motor actin | Cell structure | Over- | 2.81 | P < 0.05 |
| (actin filament) | | proteins | binding protein | | expressed | | |
| muscle Z-line, | | | | | | | |
| alpha 3 | | | | | | | |
| CASP8 and | CFLAR | Protease | Cysteine protease | Proteolysis/ | Under- | 0.38 | P < 0.01 |
| FADD-like | | | | Apoptosis | expressed | | |
| apoptosis | | | | | | | |
| regulator | | | | | | | |
| Cell division cycle | CCAR1 | Miscellaneous | Calmodulin related | Oncogenesis | Under- | 0.55 | P < 0.05 |
| and apoptosis | | function | protein | | expressed | | |
| regulator 1 | | | | | | | |
| Cell division cycle | CDC2 | Receptor | Serine/threonine | Cell cycle control | Under- | 0.18 | P < 0.05 |
| 2, G1 to S and | | | protein kinase | | expressed | | |
| G2 to M | | | receptor | | | | |
| Chemokine | CCL16 | Signalling | Chemokine | Ligand-mediated | Under- | 0.16 | P < 0.01 |
| (C—C motif) | | molecule | | signalling/ | expressed | | |
| ligand 16 | | | | Cytokine and | | | |
| | | | | chemokine | | | |
| | | | | mediated | | | |
| | | | | signaling pathway | | | |
| | | | | and immunity | | | |
| Chondroitin | CSPG6 | Nucleic acid | Chromatin/ | DNA replication | Under- | 0.36 | P < 0.05 |
| sulfate | | binding | Chromatin- | and repair/ | expressed | | |
| proteoglycan 6 | | | binding protein | Chromatin | | | |
| (bamacan) | | | | packaging | | | |
| | | | | and remodelling/ | | | |
| | | | | Chromosome | | | |
| | | | | segregation | | | |
| Cofilin | CFLP1 | Cytoskeletal | Non-motor actin | Cell structure | Over- | 2.69 | P < 0.05 |
| pseudogene 1 | | protein | binding protein | | expressed | | |
| Colony | CSF1R | Kinase | Protein kinase/ | Cell surface | Under- | 0.48 | P < 0.05 |
| stimulating factor | | | Tyrosine protein | receptor mediated | expressed | | |
| 1 receptor, | | | kinase receptor | signal | | | |
| formerly | | | | transduction/ | | | |
| McDonough | | | | Receptor protein | | | |
| feline sarcoma | | | | tyrosine kinase | | | |
| viral (v-fms) | | | | signaling | | | |
| oncogene | | | | pathway; | | | |
| homolog | | | | Immunity and | | | |
| | | | | defense/Cell | | | |
| | | | | proliferation and | | | |
| | | | | differentiation | | | |
| Cyclin-dependent | CDK4 | Kinase | Non-receptor | Mitosis/Protein | Under- | 0.46 | P < 0.01 |
| kinase 4 | | | serine/threonine | phosphorylation/ | expressed | | |
| | | | protein kinase | Cell cycle control | | | |
| Defective in sister | DSCC1/ | Chromatin | Replication origin | DNA replication/ | Under- | 0.20 | P < 0.01 |
| chromatid | DCC1 | binding | binding protein | Cell proliferation | expressed | | |
| cohesion homolog | | | | and differentiation | | | |
| 1 (S. cerevisiae) | | | | | | | |
| DEAH | DHX9 | Helicase | RNA helicase | mRNA splicing | Under- | 0.18 | P < 0.01 |
| (Asp-Glu-Ala-His) | | | | | expressed | | |
| box | | | | | | | |
| polypeptide 9 | | | | | | | |
| Discoidin, CUB | DCBLD1 | Oxidoreductase | Enzyme regulator | Signal | Under- | 0.23 | P < 0.01 |
| and LCCL domain | | | activity | transduction/cell- | expressed | | |
| containing 1 | | | | cell signalling | | | |
| EPH receptor B2 | EPHB2 | Receptor | Tyrosine protein | Cell proliferation | Under- | 0.24 | P < 0.05 |
| | | | kinase receptor | and | expressed | | |
| | | | | differentiation/ | | | |
| | | | | Protein | | | |
| | | | | phosphorylation/ | | | |
| | | | | Receptor protein | | | |
| | | | | tyrosine kinase | | | |
| | | | | signaling pathway | | | |
| ERO1-like protein | ERO1L | Oxidoreductase | Oxidoreductase | Protein folding/ | Over- | 2.21 | P < 0.05 |
| alpha | | | | Electron | expressed | | |
| | | | | transport; | | | |
| | | | | Intracellular | | | |
| | | | | protein | | | |
| | | | | traffic/General | | | |
| | | | | vesicle transport | | | |
| F-box protein 10 | FBXO10 | Ligase | Ubiquitin-protein | Protein metabolic | Under- | 0.27 | P < 0.05 |
| | | | ligase activity | process | expressed | | |
| Gamma- | GABRB2 | Receptor | GABA receptor/ | Ligand-mediated | Over- | 2.45 | P < 0.05 |
| aminobutyric acid | | | Ion channel | signalling/Anion | expressed | | |
| (GABA) A | | | | transport | | | |
| receptor, beta 2 | | | | | | | |
| Golgi membrane | GOLPH2/ | Membrane traffic | Molecular function | Biological process | Under- | 0.39 | P < 0.01 |
| protein 1 | GOLM1 | | unclassified | unclassified | expressed | | |
| Heat shock | HSPB6 | Chaperone | Other chaperones | Stress response/ | Under- | 0.61 | P < 0.05 |
| protein, alpha- | | | | Protein folding | expressed | | |
| crystallin-related, B6 | | | | | | | |
| Kallikrein-related | KLK8 | Protease | Serine protease | Proteolysis/Cell | Under- | 0.39 | P < 0.01 |
| peptidase 8 | | | | cycle control/Cell | expressed | | |
| | | | | proliferation and | | | |
| | | | | diffentiation/ | | | |
| | | | | Neurogenesis | | | |
| La | LARP2 | Ribonucleoprotein | RNA binding | Nucleobase, | Under- | 0.37 | P < 0.01 |
| ribonucleoprotein | | | | nucleoside, | expressed | | |
| domain family, | | | | nucleotide and | | | |
| member 2 | | | | nucleic acid | | | |
| | | | | metabolic process | | | |
| Mov1011, | MOV10L1 | Helicase | Helicase | Cell cycle/Protein | Under- | 0.19 | P < 0.05 |
| Moloney | | | | metabolic process | expressed | | |
| leukaemia virus | | | | | | | |
| 10-like 1, | | | | | | | |
| homolog (mouse) | | | | | | | |
| Myotrophin | MTPN | Oxidoreductase | Molecular function | Biological process | Under- | 0.31 | P < 0.01 |
| | | | unclassified | unclassified | expressed | | |
| Nuclear receptor | NRIP3 | Protease | Transcription | mRNA | Under- | 0.28 | P < 0.01 |
| interacting protein | | | cofactor | transcription | expressed | | |
| 3 | | | | regulation | | | |
| Nucleosome | NAP1L4 | Phosphatase | Phosphatase | Chromatin | Under- | 0.42 | P < 0.05 |
| assembly protein | | | inhibitor | packaging and | expressed | | |
| 1-like 4 | | | | remodelling/ | | | |
| | | | | Apoptosis/DNA | | | |
| | | | | replication | | | |
| OTU domain | OTUD5 | Nucleic acid | Nucleic acid | Chromosome | Under- | 0.24 | P < 0.01 |
| containing 5 | | binding | Binding/other | segregation | expressed | | |
| | | | protease | | | | |
| Pogo transposable | POGZ | Nulceic acid | Other nucleic | Other mRNA | Under- | 0.06 | P < 0.05 |
| element with ZNF | | binding | acid binding | transcription/ | expressed | | |
| domain | | | | Developmental | | | |
| | | | | processes | | | |
| Potassium | KCNT2 | Ion channel | Voltage-gated | Cation transport | Under- | 0.37 | P < 0.05 |
| channel, | | | potassium channel | | expressed | | |
| subfamily T, | | | | | | | |
| member 2 | | | | | | | |
| Retinoblastoma | RBBP6 | Nuclease | Nuclease | mRNA | Under- | 0.15 | P < 0.01 |
| binding protein 6 | | | | polyadenylation/ | expressed | | |
| | | | | Other metabolism | | | |
| Retinoid-binding | RBP7 | Transfer/carrier | Other | Lipid and fatty | Under- | 0.33 | P < 0.05 |
| protein 7 | | protein | transfer/carrier | acid transport/ | expressed | | |
| | | | protein | Vitamin/cofactor | | | |
| | | | | transport/Steroid | | | |
| | | | | hormone- | | | |
| | | | | mediated | | | |
| | | | | signaling/ | | | |
| | | | | Transport | | | |
| RNA | RPUSD3 | Lyase | Synthase/Lyase | rRNA | Under- | 0.37 | P < 0.05 |
| pseudouridylate | | | | metabolism | expressed | | |
| synthase domain | | | | | | | |
| containing 3 | | | | | | | |
| Septin 11 | Septin 11 | Cytoskeletal | Small GTPase | Cytokinesis | Under- | 0.23 | P < 0.01 |
| | | protein | | | expressed | | |
| SET binding | SBF2 | Phosphatase | Other | Phospholipid | Under- | 0.49 | P < 0.05 |
| factor 2 | | | phosphatase | metabolism/ | expressed | | |
| | | | | General vesicle | | | |
| | | | | transport | | | |
| Solute carrier | SLC12A2 | Transporter | Other transporter/ | Cation transport | Under- | 0.19 | P < 0.05 |
| family 12 | | | Cation transporter | | expressed | | |
| (sodium/potassium/ | | | | | | | |
| chloride | | | | | | | |
| transporters), | | | | | | | |
| member 2 | | | | | | | |
| Sorting nexin 10 | SNX10 | Membrane traffic | Membrane traffic | Intracellular | Over- | 2.52 | P < 0.05 |
| | | | regulatory protein | protein traffic | expressed | | |
| splA/ryanodine | SPSB2 | Signalling | Signalling molecule | Cell surface | Under- | 0.23 | P < 0.01 |
| receptor domain | | molecule | | receptor linked | expressed | | |
| and SOCS box | | | | signal | | | |
| containing 2 | | | | transduction/ | | | |
| | | | | Intracellular | | | |
| | | | | signaling cascade/ | | | |
| | | | | Ubiquitination | | | |
| Stearoyl-CoA | SCD5 | Oxidoreductase | Other | Fatty acid | Over- | 1.61 | P < 0.05 |
| desaturase 5 | | | oxidoreductase | metabolism | expressed | | |
| Suppression of | ST13 | Chaperone | Chaperone | Protein folding | Under- | 0.53 | P < 0.05 |
| tumourigenicity | | | | | expressed | | |
| 13 (colon carcinoma) | | | | | | | |
| (Hsp70 interacting | | | | | | | |
| protein) | | | | | | | |
| SWAP-70 protein | SWAP70 | Nucleic acid | Other nucleic acid | B-cell- and a | Under- | 0.31 | P < 0.05 |
| | | binding | binding | ntibody-mediated | expressed | | |
| | | | | immunity/DNA | | | |
| | | | | recombination | | | |
| Synaptotagmin | SYT16 | Membrane traffic | Membrane traffic | Exocytosis | Over- | 1.45 | P < 0.05 |
| XVI | | | regulatory protein | | expressed | | |
| Synaptotagmin | SYT9 | Membrane traffic | Membrane traffic | Neurotransmitter | Under- | 0.36 | P < 0.01 |
| IX | | | regulator protein | release/Regulated | expressed | | |
| | | | | exocytosis | | | |
| Transmembrane | TMEM57 | Cytoskeletal | Actin binding | Neurogenesis/ | Under- | 0.30 | P < 0.05 |
| protein 57 | | protein | cytoskeletal protein/ | Chromosome | expressed | | |
| | | | Other microtubule | segregation | | | |
| | | | family cytoskeletal | | | | |
| | | | protein | | | | |
| Tropomyosin 2 | TPM2 | Cytoskeletal | Actin binding | Cell motility/Cell | Under- | 0.46 | P < 0.01 |
| (beta) | | protein | motor protein | structured/Muscle | expressed | | |
| | | | | contraction/ | | | |
| | | | | Muscle | | | |
| | | | | development | | | |
| Tumour-associated | TACSTD2 | Receptor | Receptor | Cell surface | Under- | 0.06 | P < 0.01 |
| calcium signal | | | | receptor mediated | expressed | | |
| transducer 2 | | | | signal | | | |
| | | | | transduction/Cell | | | |
| | | | | proliferation and | | | |
| | | | | differentiation | | | |
| Tumour necrosis | TNFSF7 | Signalling | Cytokine | Ligand-mediated | Over- | 2.18 | P < 0.05 |
| factor (ligand) | | molecule | | signaling/ | expressed | | |
| superfamily, | | | | B-cell-and | | | |
| member 7 | | | | antibody-mediated | | | |
| | | | | immunity | | | |
| Tumor protein | TP53I3 | Oxidoreductase | Dehydrogenase | Carbohydrate | Under- | 0.48 | P < 0.01 |
| p53 inducible | | | | metabolism/ | expressed | | |
| protein 3 | | | | Apoptosis | | | |
| Tumour protein, | TPT1 | Cytoskeletal | Non-motor | Immunity and | Over- | 2.06 | P < 0.05 |
| translationally- | | protein | microtubule | defense | expressed | | |
| controlled 1 | | | binding protein | | | | |
| UDP | UGT1A8 | Transferase | Glycosyltransferase | Other | Under- | 0.16 | P < 0.05 |
| glucuronosyltransferase | | | | polysaccharide | expressed | | |
| 1 family, | | | | metabolism/ | | | |
| polypeptide | | | | Steroid hormone | | | |
| A9; UDP | | | | metabolism | | | |
| glucuronosyltransferase | | | | | | | |
| 1 family, | | | | | | | |
| polypeptide | | | | | | | |
| A5; UDP | | | | | | | |
| glucuronosyltransferase | | | | | | | |
| 1 family, | | | | | | | |
| polypeptide | | | | | | | |
| A10; UDP | | | | | | | |
| glucuronosyltransferase | | | | | | | |
| 1 family, | | | | | | | |
| polypeptide | | | | | | | |
| A6; UDP | | | | | | | |
| glucuronosyltransferase | | | | | | | |
| 1 family, | | | | | | | |
| poly | | | | | | | |
| Uveal autoantigen | UACA | Transfer/carrier | Unclassified | Unclassified | Under- | 0.16 | P < 0.05 |
| with coiled-coil | | protein | | | expressed | | |
| domains and | | | | | | | |
| ankyrin repeats | | | | | | | |
| Vacuolar protein | VPS25 | Membrane traffic | Unclassified | Nucleoside, | Under- | 0.29 | P < 0.05 |
| sorting 25 | | | | nucleotide and | expressed | | |
| (S. Cerevisiae) | | | | nucleic acid | | | |
| | | | | metabolism/ | | | |
| | | | | Protein | | | |
| | | | | metabolism and | | | |
| | | | | modification/ | | | |
| | | | | Intracellular | | | |
| | | | | protein traffic/ | | | |
| Zinc finger E-box | TCF8/ | KRAB box | KRAB box | DNA binding/ | Under- | 0.14 | P < 0.05 |
| binding | ZEB1 | transcription | transcription | transcription | expressed | | |
| homeobox 1 | | factor | factor | factor activity | | | |
| ZW10 | ZW10 | Cytoskeletal | Other microtubule | Developmental | Under- | 0.50 | P < 0.05 |
| kinetochore | | protein | family cytoskeletal | processes/ | expressed | | |
| associated, | | | protein | Chromosome | | | |
| homolog | | | | segregation | | | |
| (Drosophila) | | | | | | | |

TLDA analyses took place as described previously (Fragouli et al., 2010). The customized TLDA cards consisted of 384 (4×96) wells pre-loaded with specified sequence detection (TaqMan) probes. The probes included were chosen based upon the results of a previous series of gene expression microarray experiments. The microarray analyses had assessed the relative expression levels of ˜30,000 mRNA transcripts in CCs surrounding normal and aneuploid oocytes and highlighted 729 genes that displayed apparent alterations in transcript number in CCs associated with chromosomally abnormal oocytes (data not shown). Ninety four of these genes were selected for further analysis using TLDAs (Table 9). Additionally, two house-keeping genes, glyceraldhyde-3-phosphate dehydrogenase (GADPH) and hypoxanthinine phopsphoribosyltransferase 1 (HPRT1) were examined.

Pre-amplification of cDNA samples took place in a thermocycler, using a pool of all 96 TaqMan assays and TaqMan Preamp Master Mix (×2) (Applied Biosystems, UK). Reactions were prepared as suggested by the manufacturer and pre-amplification occurred under the following conditions: 10 min hold at 95° C. followed by 14 cycles of 15 sec at 95° C. and 4 minutes at 60° C. 2× TaqMan Gene Expression Master Mix (Applied Biosystems, UK) was used during the final real-time PCR step, which took place using the following set of conditions: 50° C. for 2 minutes, 95° C. for 10 minutes, and 40 cycles of 95° C. for 15 sec and 60° C. for 1 minute. ΔCt values were determined by normalization to both house-keeping genes, using the RQ SDS manager software (Applied Biosystems, UK). Replicate analyses were performed for every sample, such that the expression of all 96 genes were assessed in each sample four times.

### CC Gene Expression Analysis Via Real-Time PCR

A set of smaller scale real-time PCR experiments was used to examine the expression of three genes in the 26 additional samples composed of the inner layer of CCs from oocytes with aCGH PB analysis (Tables 6 and 7). The examined genes included the tumor protein p53 inducible protein 3 (TP53I3), the splA/ryanodine receptor domain and SOCS box containing 2 (SPSB2), and the housekeeping gene HPRT1. Real-time PCR took place with the use of pre-designed TaqMan gene expression assays (Applied Biosystems, UK) which were available for all three genes (TP53I3: Hs00936520_m1; SPSB2: Hs00261880_m1; HPRT1: Hs01003267_m1). Triplicate amplification reactions were set-up for all three genes in each of the 26 CC samples. Each reaction contained 2 μl of cDNA, 7 μl of nuclease-free H₂O, 10 μl of Taq-Man Gene Expression Mastermix (Applied Biosystems, UK), and 1 μl of the 20× Taq-Man Gene expression Assay (Applied Biosystems, UK), for a total volume of 20 μl. The thermal cycler used was a StepOne Real Time PCR System (Applied Biosystems, UK), and the following conditions were employed: incubation at 50° C. for 2 minutes, incubation at 95° C. for 10 minutes, and then 45 Cycles of 95° C. for 15 sec and 60° C. for 1 minute.

### Statistical Analysis

The TLDA data obtained were first normalized against the two housekeeping genes GADPH and HPRT1. Subsequently, RealTime StatMiner™ version 3 software (Integromics™ SL, Spain) was employed for TLDA data analysis. Simple t-tests were also used to compare the expression of the 94 selected genes in CCs coming from normal and aneuploid oocytes. Additionally, a two-tailed t-test was employed when the expression of TP53I3 and SPSB2 was compared between CCs derived from aneuploid oocytes and normal oocytes. The same approach was used to assess the expression of these two genes in CCs associated with oocytes that led to live births and those that failed to implant. For the smaller scale real-time PCR experiments, the ΔCt values for TP53I3 and SPSB2 were determined by normalizing against the housekeeping gene (HPRT1), and were further analyzed with the use of the StepOne Software v2.1 (Applied Biosystems, UK).

### Results

### Cytogenetic Analysis of Polar Bodies

Microarray CGH (aCGH) was employed for the comprehensive cytogenetic examination of a total of 26 first PBs. The corresponding metaphase II oocytes were generated by six women whose average age was 39 years (age range: 30-46 years). These women were undergoing ART procedures either due to male factor or tubal infertility (i.e. no ovarian involvement was present) with or without PGS. The obtained molecular karyotypes were used to separate the CC clumps (outer and/or inner layers) into two groups, one including samples removed from metaphase II oocytes characterized as haploid normal (23,X), and another including samples removed from chromosomally abnormal oocytes.

Table 8 shows the cytogenetic results obtained after the aCGH analysis of the 26 first PBs. A total of 10 first PBs and therefore their corresponding oocytes were characterized as being aneuploid, with the remainder being classified as normal. Thorough analysis of the aCGH data confirmed the presence of two separate mechanisms leading to aneuploidy of female origin, whole-chromosome non-disjunction and unbalanced chromatid predivision. Nine of the 22 errors scored were due to the malsegregation of entire chromosomes while the remainder were attributed to the unbalanced predivision of single chromatids. Chromosomes of all sizes were found to participate in aneuploidy events, and losses as well as gains were seen.

TLDA Analysis of Outer Cumulus Cell Layers from Normal and Aneuploid Oocytes

Gene expression in a set of 13 outer CC layers was examined. Seven clumps of CCs were removed from metaphase II oocytes whose molecular karyotypes was shown to be 23,X (normal) via aCGH (age range 36-46, mean 40.0), whereas the remaining 6 CC clumps were associated with aneuploid oocytes (age range 36-46, mean 39.7). Details of the chromosome errors scored in the corresponding first PBs are shown in Table 8. These oocytes were generated by four of the participating patients (24, 25, 26 and 28 in Table 8).

Comparisons took place to determine if the chromosomal status of the oocytes affected the quantities of specific gene transcripts in CCs. Of the 94 selected genes, 58 showed expression profiles which were confirmatory of the initial microarray data. Forty nine of the 58 genes were under-expressed in the CCs removed from aneuploid oocytes, whereas the remainder were over-expressed. In many cases the effect of chromosome errors appeared to be relatively weak, however, two genes not only showed a significant difference in expression associated with oocyte aneuploidy (P<0.05), but also had relatively large mRNA copy number fluctuations (FC>2-fold). Both of these genes, TP53I3 and SPSB2, were under-expressed in the CCs surrounding aneuploid oocytes. Classification of both these genes according to molecular function and biological process was accomplished using the PANTHER tool (Applied Biosystems), a database which contains a reference list of all known human genes. Hence, TP53I3 was classified as a dehydrogenase involved in the regulation of carbohydrate metabolism and apoptosis, whereas SBSB2 was classified as a signalling molecule participating in cell-surface mediated signal transduction, intracellular signalling and ubiquitination.

Real-Time Time PCR Analysis of Inner Cumulus Cell Layers from Normal and Aneuploid Oocytes

To further investigate the expression of both TP53I3 and SPSB2 in CCs of normal and chromosomally abnormal oocytes, a series of more focused real-time PCR experiments were carried out, concentrating on analysis of the layers of CCs closest to the oocyte. Results were obtained for a total of 21 inner CC layers, 14 of which were removed from chromosomally normal metaphase II oocytes (mean female age 38.6 years), and the remaining 7 from aneuploid oocytes (mean female age 40.1 years). Delta Ct's (ΔCt) were calculated by subtracting the Ct of either TP53I3 or SPSB2 from the Ct of the house-keeping HPRT1 gene for each of the CC samples. These ΔCt values are shown in Table 9, and for the CCs of aneuploid oocytes ranged from 0.5-2.6 for TP53I3 and 4.1-6.9 for SPSB2, while for the CCs of the chromosomally normal oocytes they varied from −1.2 to 2.2 for TP53I3 and 1.2-7.1 for SPSB2. Lower ΔCt values correspond to higher levels of gene expression, whereas high ΔCt values are associated with lower numbers of mRNA transcripts.

The real-time PCR data was concordant with the results obtained from outer CC layers obtained using TLDAs. Both genes were down-regulated in the inner CC layers of aneuploid oocytes, and up-regulated in 8 of the 14 inner CC layers taken from normal metaphase II oocytes. There were, however, 6 CC samples (7-1, 7-3, 25-2, 25-4, 26-3 and 28-1) removed from apparently normal oocytes, for which both SPSB2 and TP53I3 had an expression profile similar to that seen for CCs associated with aneuploid oocytes. The expression patterns seen for SPSB2 and TP53I3 in CCs of normal and aneuploid oocytes are illustrated in the graph of FIG. 1. Although cumulus cell gene expression analysis was not able to achieve a perfect classification of the corresponding oocytes into ‘normal’ and ‘aneuploid’ categories, a degree of separation was achieved. In the case of TP53I3, expression tended to be significantly lower amongst CCs associated with aneuploid oocytes (P=0.01). A non-significant trend towards lower expression was observed for SPSB2 (see FIG. 9).

### Cumulus Cell Expression of SPSB2 and TP53I3 and Clinical Outcome

As well as assessing the expression of SPSB2 and TP53I3 in relation to oocyte aneuploidy, we were also able to evaluate whether mRNA transcript levels for these two genes were associated with clinical outcome (i.e. the establishment of a pregnancy leading to a healthy live birth). The expression of SPSB2 and TP53I3 was assessed in a new set of 38 outer CC layers, which were examined using TLDAs. The 38 CC samples were derived from oocytes which produced embryos that were transferred without any chromosome screening. The embryos were generated by 22 women (patients 1-6 and 8-23 in Table 7). All cycles involved either single embryo transfer, transfer of two embryos followed by no pregnancy, or transfer of two embryos followed by birth of dizygotic twins. Thus the ultimate fate of each transferred embryo was known. Analysis of gene expression and outcome took place in a blinded manner. A total of 18 of the transferred embryos implanted successfully and resulted in healthy live births (mean maternal age was 32.0 years), whilst no pregnancy ensued after the transfer of the remaining 20 embryos (mean female age was 30.4 years). The day of transfer varied depending on embryo quality from days 3 to 5.

The expression profiles of TP53I3 and SPSB2 were compared between the CCs of embryos which led to live births and those who failed to implant. Of the two genes, SPSB2 showed the clearest association with successful pregnancy, tending to be up-regulated in the CCs of oocytes which led to live births. These differences in expression between the two sample groups approached statistical significance (P=0.054 two tailed t-test), and are illustrated in the box plot shown in FIG. 8.

### Discussion

The combination of expression microarrays and a new innovation in real-time PCR (TLDAs) provided for the first time a detailed insight into the follicular microenvironment of oocytes that become aneuploid. It was evident from the obtained data that the cumulus cells of the aneuploid oocytes tend to be less transcriptionally active and not as proliferative compared to the CCs of the normal oocytes.

Cumulus cells are biologically distinct from other follicular cells and perform the specialized role of supporting oocyte growth and maturation, via the transmission of signals and the supply of nutrients and other bio-molecules. The down-regulation of genes involved in transcription and protein translation would likely lead to insufficient cellular proliferation. An example is the DHX9 gene, a transcriptional co-activator that functions as a bridging factor between transcription factors/co-factors by binding to promoter sequences. It is also intimately involved in several transcriptional/translational processes relevant to the role of cumulus cells in oocyte support during folliculogenesis, including the NF-kappaB-dependent transcription. Fuller-Pace (2006) Nucleic Acids Res. 34: 4206-1215. The reduced activity of such a generic activator in cumulus cells surrounding aneuploid oocytes suggests that many cellular pathways are probably dysfunctional; consequently the cumulus cells may be unable to perform their supportive roles effectively.

The presence of aneuploidy in the enclosed oocyte had an effect on several metabolic, intracellular and membrane transport processes in the corresponding cumulus cells. Specifically, both the microarray and the TLDA analysis demonstrated that the B3GALNT2 gene was down-regulated in the cumulus cells of aneuploid oocytes. This particular gene is a glycosyltransferase, which is found in the ovary and is involved in the regulation of protein metabolism. Hiruma, et al. (2004) J. Biol. Chem 279: 14087-14095. Again, this indicates that synthetic processes are, in general, down-regulated in cumulus cells associated with abnormal oocytes.

Additionally, genes involved in pathways related to hypoxia and apoptosis were abnormally expressed providing evidence for a link between follicular microenvironment, specifically levels of oxygenation, and aneuploidy. Examples include SEPTIN 11 and SPSB2. SEPTIN 11 belongs to the GTPase super-class of P-loop NTPases and interacts with various proteins involved in apoptosis. Nakahira, et al. (2010) PLoS One 5: e13799. SPSB2, on the other hand, belongs to the SOCS box family of E3 ubiquitin ligases and functions as an adaptor protein in the E3 ubiquitin ligase complex that ubiquitinates the inducible form of nitric oxide synthase (iNOS), targeting it for proteosomal degradation. Kuang, et al. (2010) J. Cell Biol. 190: 129-141. In cumulus cells associated with aneuploid oocytes, the reduced levels of mRNA transcripts of these two genes involved in ubiquitination and apoptosis may result in the accumulation of excessive levels of abnormal/redundant proteins or the formation of reactive oxygen species, i.e. from raised levels of nitric oxide due to over-activity of iNOS, but also in the reduced apoptotic capability of these defective cumulus cells.

Of the 94 genes the expression of which was further validated with the use of TLDAs, 14 genes identified showed consistent, highly significant, differences in expression (e.g., B3GALNT2, C22orf29, CCL16, DCBLD1, DCC1, DHX9, OTUD5, RBBP6, SEPT11, SLC25A36, SPSB2, TACSTD2, Unassigned, and TP53I3). The SPSB2 and TP53I3 genes are of particular interest. A blinded study of these genes showed that they display differential expression associated with the ability of the corresponding oocyte to produce, after fertilization, a healthy live birth. Although aneuploidy may well be the single most important determinant of oocyte competence, responsible for many cases of implantation failure or miscarriage, there are other factors that also contribute to oocyte potential. Consequently it is expected that some oocytes that are chromosomal)/normal will nonetheless fail to produce a viable pregnancy. Given this confounding effect, the fact that an apparent difference in SPSB2 and TP53i3 expression related to live birth was seen is striking.

In conclusion, there is strong evidence from independent microarray and real-time PCR studies that the subset of the genes reported here can be used for a non-invasive assessment of oocyte aneuploidy. Furthermore, an additional, blinded, analysis suggests that data obtained using this approach could be predictive of live birth. This strategy may allow a reduction in, or elimination of, polar body and/or blastomere biopsy for the assessment of aneuploidy. Given that analysis of CCs can be completed within 4 hours, it should be possible to reveal abnormal oocytes before fertilization, restricting fertilization to those oocytes found to be chromosomally normal, reducing ethical concerns over embryo/oocyte screening. Alternatively analysis could be completed after fertilization. Analyses using this approach would cost a small fraction of alternative invasive (e.g., aCGH) methods and carry no risk to the oocyte or embryo.

The data provided herein shed light on pathways with atypical activity in CCs associated with abnormal oocytes. Without being bound to a particular theory, it is not yet clear whether these changes are induced by the abnormal oocyte itself or are a consequence of a poor follicular microenvironment, which is also responsible for predisposing the oocyte to chromosome malsegregation. However, the identification of these genes and pathways offers the possibility of finally understanding the genesis of female meiotic aneuploidy and perhaps even providing targets for interventions, either in vivo or during in vitro oocyte maturation, aimed at lessening the risk of aneuploidy occurring.

Although the invention has been described in some detail by way of illustration and example for purposes of clarity of understanding, it will be obvious that certain changes and modifications will practiced within the scope of the appended claims. Modifications of the above-described modes for carrying out the invention that are obvious to persons of skill in medicine, pharmacology, microbiology, and/or related fields are intended to be within the scope of the following claims.

All publications (e.g., Non-Patent Literature), patent application publications, and patent applications mentioned in this specification are indicative of the level of skill of those skilled in the art to which this invention pertains. All such publications (e.g., Non-Patent Literature), patent application publications, and patent applications are herein incorporated by reference to the same extent as if each individual publication, patent, patent application publication, or patent application is specifically and individually indicated to be incorporated by reference.

What is claimed:

1. A method of evaluating the competence of a mammalian oocyte for implantation or fertilization comprising:
(i) obtaining a nucleic acid or polypeptide sample;
(ii) determining the level of marker expression of at least one gene or corresponding polypeptide encoded thereby selected from the group of TP53I3 or SPSB2 in said sample; and
(iii) comparing the level of marker expression TP53I3 or SPSB2 in the sample with a control or reference standard,
wherein detecting differential marker expression between the sample and the control is indicative of the competence of an oocyte for implantation.

2. The method of claim 1 wherein said TP53I3 gene is a human or non-human primate gene or polypeptide.

3. The method of claim 1 or 2, wherein said TP53I3 gene has the nucleic acid sequence of SEQ ID NO: 385, 387, or 389, or comprises the amino acid sequence of SEQ ID NO: 386, 388, or 390.

4. The method of claim 1 wherein said SPSB2 gene is a human or non-human primate gene or polypeptide.

5. The method of claim 1 or 4, wherein SPSB2 has the nucleic acid sequence of SEQ ID NO: 252 or comprises the amino acid sequence of SEQ ID NO: 351.

6. The method of any one of claims 1-5, wherein the sample is derived from an oocyte or cumulus cell.

7. The method of any one of claims 1-6, wherein the control or reference standard is derived from one of the group consisting of: an oocyte competent for implantation, a chromosomally normal oocyte, an oocyte not competent for implantation, and a chromosomally abnormal oocyte.

8. The method of any one of claims 1-6, wherein the sample is derived from follicular fluid.

9. The method of any one of claims 1-6, wherein the sample is derived from one or more cumulus cells of an oocyte potentially to be used for IVF implantation.

10. The method of any one of claims 1-6, wherein the control or reference standard is derived from one of the group consisting of: follicular fluid associated with an oocyte competent for implantation, follicular fluid associated with a chromosomally normal oocyte, follicular fluid associated with an oocyte not competent for implantation and follicular fluid associated with a chromosomally abnormal oocyte.

11. The method of any one of claims 1-6, wherein the sample is derived from culture medium.

12. The method of any one of claims 1-6, wherein the control or reference standard is derived from one of the group consisting of: culture medium associated with an oocyte competent for implantation, culture medium associated with a chromosomally normal oocyte, culture medium associated with an oocyte not competent for implantation, and culture medium associated with a chromosomally abnormal oocyte and the assay detects the level of expression of both TP53I3 and SPSB2.

13. The method of any one of claims 1-12, wherein the level of marker expression determined in the sample is at least 20% different from the level of marker expression determined in the control or reference standard.

14. The method of any one of claims 1-13 wherein the level of marker expression is detected by at least one of the group consisting of nucleic acid microarray, Northern blot, and reverse transcription PCR.

15. The method of any one of claims 1-14, wherein the level of marker expression is detected by at least one of the group consisting of Western blot, enzyme-linked immunosorbent assay, protein microarray and FACS analysis.

16. The method of any one of claims 1-15, wherein the mammalian oocyte is of a domesticated mammal.

17. The method of claim 1, wherein the mammalian oocyte is of a human.

18. The method of any one of claims 1-17 that further includes detecting at least one other nucleic acid or polypeptide encoded thereby selected from the group consisting of SEQ ID NO:1-390 and the corresponding encoded polypeptide or any combination thereof.

19. The method of claim 18, that further includes: (i) determining in a sample the level. of marker expression of at least one nucleic acid or corresponding polypeptide encoded thereby selected from the nucleic acids exemplified by SEQ ID NOS: 184, 187, 189, 191, and 230, and (ii) comparing the level of marker expression in the sample with a control or reference standard, wherein detecting differential marker expression of said nucleic acids or polypeptides indicative of the competence of an oocyte for implantation.

20. The method of claim 18 or 19, wherein the sample is derived from a cumulus cell.

21. The method of claim 18 or 19, wherein the control or reference standard is derived from one of the group consisting of a cumulus cell associated with an oocyte competent for implantation, a cumulus cell associated with a chromosomally normal oocyte, a cumulus cell associated with an oocyte not competent for implantation and a cumulus cell associated with a chromosomally abnormal oocyte, or the sample is derived from follicular fluid.

22. The method of claim 18 or 19, wherein the control or reference standard is derived from one of the group consisting of: culture medium associated with a cumulus cell associated with an oocyte competent for implantation, culture medium associated with a cumulus cell associated with a chromosomally normal oocyte, culture medium associated with a cumulus cell associated with an oocyte not competent for implantation, and culture medium associated with a cumulus cell associated with a chromosomally abnormal oocyte.

23. The method of any one of claims 1-22 wherein the mammalian oocyte is of a domesticated mammal or is human oocyte.

24. The method of claim 23, wherein the mammalian oocyte is a human oocyte.

25. The method of any one of claims 1-24, wherein the method further includes
(i) determining in a sample the level of marker expression of at least one nucleic acid selected from the group of nucleic acids exemplified by SEQ ID NOS: 19, 25, 33, 38, and 43 and (ii) comparing the level of marker expression in the sample with a control or reference standard, wherein detecting differential marker expression between the sample and the control is indicative of the competence of the oocyte for implantation or fertilization.

26. The method of any one of claims 1-25, that further includes determining the level of marker expression of at least one nucleic acid that encodes an amino acid sequence selected from the group of amino acids consisting of SEQ ID NOS: 183-282 in said sample; and comparing the level of marker expression in the sample with a control or reference standard, wherein detecting differential marker expression between the sample and the control is indicative of the competence of the oocyte for fertilization.

27. The method of any one of claims 1-26, that further includes evaluating the competence of a mammalian oocyte for fertilization by in addition determining the level of marker expression of at least one amino acid selected from the group of amino acids consisting of SEQ ID NOS: 283-390 in said sample; and
comparing the level of marker expression in the sample with a control or reference standard, wherein detecting differential marker expression between the sample and the control is indicative of the competence of the oocyte for fertilization.

28. The method of any one of claims 1-27, wherein the level of marker expression is detected by nucleic acid microarray, real time PCR, Northern blot, or reverse transcription PCR.

29. The method of any one of claims 1-27, wherein the level of marker expression is detected by Western blot, immunoassay (e.g., enzyme-linked immunosorbent assay), protein microarray, or FACS analysis.

30. The method off any one of claims 1-29, that in addition includes determining the level of marker expression of at least one gene selected from the group consisting of B3GALNT2, C22orf29, CCL16, DCBLD1, DCC1, DHX9, OTUD5, RBBP6, SEPT11, SLC25A36, and TACSTD2, Unassigned (helicase) in said sample; and
comparing the level of marker expression in the sample with a control or reference standard, wherein detecting differential marker expression between the sample and the control is further indicative of the competence of the oocyte for implantation or fertilization.

31. The method of any one of claims 1-30, wherein the sample is derived from a cumulus cell, an oocyte, polar body, is a nucleic acid sample or is an amino acid sample.

32. The method of any one of claims 1-31, wherein the control or reference standard is derived from one of the group consisting of a cumulus cell associated with an oocyte competent for implantation, a cumulus cell associated with a chromosomally normal oocyte, a cumulus cell associated with an oocyte not competent for implantation and a cumulus cell associated with a chromosomally abnormal oocyte.

33. The method of any one of claims 1-32, wherein the sample is derived from follicular fluid.

34. The method of claim 33, wherein the control or reference standard is derived from one of the group consisting of follicular fluid associated with a cumulus cell associated with an oocyte competent for implantation, follicular fluid associated with a cumulus cell associated with a chromosomally normal oocyte, follicular fluid associated with a cumulus cell associated with an oocyte not competent for implantation and follicular fluid associated with a cumulus cell associated with a chromosomally abnormal oocyte.

35. The method of any one of claims 1-34, wherein the sample is derived from culture medium.

36. The method of claim 35, wherein the control or reference standard is derived from one of the 36. group consisting of culture medium associated with a cumulus cell associated with an oocyte competent for implantation, culture medium associated with a cumulus cell associated with a chromosomally normal oocyte, culture medium associated with a cumulus cell associated with an oocyte not competent for implantation, and culture medium associated with a cumulus cell associated with a chromosomally abnormal oocyte.

37. The method of any one of claims 1-36, wherein the level of marker expression is detected by nucleic acid microarray, cytogenetic analysis (aCGH), real-time PCR, TLDA, Northern blot, or reverse transcription PCR and/or by Western blot, immunoassay (e.g., enzyme-linked immunosorbent assay), protein microarray, or FACS analysis.

38. The method of any one of claims 1-37, which further includes detecting B3GALNT2 the nucleic acid sequence of SEQ ID NO: 185 or the amino acid sequence of SEQ ID NO: 285.

39. The method of any one of claims 1-37, which further includes detecting the C22orf29 nucleic acid sequence of SEQ ID NO: 381 or the amino acid sequence of SEQ ID NO: 382.

40. The method of any one of claims 1-37, which further includes detecting the CCL16 nucleic acid sequence of SEQ ID NO: 187 or the amino acid sequence of SEQ ID NO: 287.

41. The method of any one of claims 1-37, which further includes detecting the DCBLD1 is nucleic acid sequence of SEQ ID NO: 191 or the amino acid sequence of SEQ ID NO: 291.

42. The method of any one of claims 1-37, which further includes detecting the DHX9 nucleic acid sequence of SEQ ID NO: 188 or the amino acid sequence of SEQ ID NO: 288.

43. The method of any one of claims 1-37, which further includes detecting the OTUD5 nucleic acid sequence of SEQ ID NO: 193 or the amino acid sequence of SEQ ID NO: 293.

44. The method of any one of claims 1-37, which further includes detecting the RBBP6 nucleic acid sequence of SEQ ID NO: 184 or the amino acid sequence of SEQ ID NO: 284.

45. The method of any one of claims 1-37, which further includes detecting the SEPT11 nucleic acid sequence of SEQ ID NO: 192 or the amino acid sequence of SEQ ID NO: 292.

46. The method of any one of claims 1-37, which further includes detecting the SLC25A36 nucleic acid sequence of SEQ ID NO: 190 or the amino acid sequence of SEQ ID NO: 290.

47. The method of any one of claims 1-37, which further includes detecting the TACSTD2 nucleic acid sequence of SEQ ID NO: 383 or the amino acid sequence of SEQ ID NO: 384.

48. An array comprising at least SPSB2 and TP53I3 genes or SPSB2 and TP53I3 nucleic acids, primers, polypeptides or antibodies which specifically detect, amplify, or bind to SPSB2 and TP53I3 nucleic acids or polypeptides.

49. The array of claim 48, that comprises primers which amplify SPSB2 and TP53I3 nucleic acids.

50. The array of claim 48, that comprises antibodies or nucleic acids which specifically bind SPSB2 and TP53I3 polypeptides.

51. The array of any of claims 48-50 that comprises nucleic acids or polypeptides that are at least 90% identical to the TP53I3 nucleic acid sequence of SEQ ID NO: 385, 387, or 389, or the amino acid sequence of SEQ ID NO: 386, 388, or 390 and/or to the SPSB2 nucleic acid sequence of SEQ ID NO: 252 and/or the amino acid sequence of SEQ ID NO: 351.

52. The array of claim 51, that comprises nucleic acids or polypeptides that are at least 95% identical to the TP53I3 nucleic acid sequence of SEQ ID NO: 385, 387, or 389, or the amino acid sequence of SEQ ID NO: 386, 388, or 390 or to the SPSB2 nucleic acid sequence of SEQ ID NO: 252 or the amino acid sequence of SEQ ID NO: 351.

53. The array of any of claim 52 that comprises nucleic acids or polypeptides that are identical to the TP53I3 nucleic acid sequence of SEQ ID NO: 385, 387, or 389, or the amino acid sequence of SEQ ID NO: 386, 388, or 390, and identical to the SPSB2 nucleic acid sequence of SEQ ID NO: 252 and/or the amino acid sequence of SEQ ID NO: 351.

54. The array of claim 48 that comprises nucleic acid primers that amplify the TP53I3 nucleic acid sequence of SEQ ID NO: 385, 387, or 389 and/or the SPSB2 nucleic acid sequence of SEQ ID NO: 252.

55. The array of any one of claims 48-54 wherein said SPSB2 gene and said TP53I3 gene are human or non-human primate genes and said nucleic acid, primer, polypeptide or antibody is a human or non-human primate gene, or a nucleic acid, primer, polypeptide or an antibody that specifically amplifies or specifically binds to said human or non-human primate gene, nucleic acid or polypeptide.

56. The array of any of claims 48-55, that comprises one or more detectable labels which may be attached to said gene, nucleic acids, polypeptides, primers or antibodies.

57. The array of claim 56, wherein SPSB2 is encoded by the nucleic acid sequence of SEQ ID NO: 252 or comprises the amino acid sequence of SEQ ID NO: 351.

58. The array of any one of claims 48-57, further comprising at least one gene, primer polypeptide or antibody that encodes, amplifies, or binds a gene selected from the group consisting of B3GALNT2, C22orf29, CCL16, DCBLD1, DCC1, DHX9, OTUD5, RBBP6, SEPT11, SLC25A36, and TACSTD2, Unassigned (helicase) or a polypeptide encoded thereby.

59. The array of claim 58, further comprising at least two, three, four, five, six, seven, eight, nine or ten or all eleven of said genes or polypeptides.

60. The array of claim 58, wherein B3GALNT2 is encoded by the nucleic acid sequence of SEQ ID NO: 185 or comprises the amino acid sequence of SEQ ID NO: 285.

61. The array of claim 58, wherein C22orf29 is encoded by the nucleic acid sequence of SEQ ID NO: 381 or comprises the amino acid sequence of SEQ ID NO: 382.

62. The array of claim 58, wherein CCL16 is encoded by the nucleic acid sequence of SEQ ID NO: 187 or comprises the amino acid sequence of SEQ ID NO: 287.

63. The array of claim 58, wherein DCBLD1 is encoded by the nucleic acid sequence of SEQ ID NO: 291 or comprises the amino acid sequence of SEQ ID NO: 291.

64. The array of claim 58, wherein DHX9 is encoded by the nucleic acid sequence of SEQ ID NO: 188 or comprises the amino acid sequence of SEQ ID NO: 288.

65. The array of claim 58, wherein OTUD5 is encoded by the nucleic acid sequence of SEQ ID NO: 193 or comprises the amino acid sequence of SEQ ID NO: 293.

66. The array of claim 58, wherein RBBP6 is encoded by the nucleic acid sequence of SEQ ID NO: 184 or comprises the amino acid sequence of SEQ ID NO: 284.

67. The array of claim 58, wherein SEPT11 is encoded by the nucleic acid sequence of SEQ ID NO: 192 or comprises the amino acid sequence of SEQ ID NO: 292.

68. The array of claim 58, wherein SLC25A36 is encoded by the nucleic acid sequence of SEQ ID NO: 190 or comprises the amino acid sequence of SEQ ID NO: 290.

69. The array of claim 58, wherein TACSTD2 is encoded by the nucleic acid sequence of SEQ ID NO: 383 or comprises the amino acid sequence of SEQ ID NO: 384.
